(19) United States
(12) Patent Application Publication
(10) Pub. No.: US 2025/0273338 A1
(43) Pub. Date: Aug. 28, 2025

(54) BIOMARKERS FOR DETECTING COLORECTAL CANCER OR COLORECTAL ADENOMA AND METHODS THEREOF

(71) Applicant: PRECOGIFY PHARMACEUTICAL CHINA CO., LTD., Beijing (CN)

(72) Inventors: Kai LIN, Beijing (CN); Xudong DAI, Beijing (CN); Yu TIAN, Beijing (CN); Tengsong CUI, Beijing (CN)

(73) Assignee: PRECOGIFY PHARMACEUTICAL CHINA CO., LTD., Beijing (CN)

(21) Appl. No.: 19/199,351

(22) Filed: May 5, 2025

Related U.S. Application Data
(63) Continuation of application No. PCT/CN2022/130380, filed on Nov. 7, 2022.

(51) Int. Cl. G16H 50/20; G06N 20/00; G16H 50/30

(52) U.S. Cl. CPC G16H 50/20; G06N 20/00; G16H 50/30

(57) ABSTRACT

A group of diagnostic biomarkers usable for diagnosis of colorectal cancer or advanced colorectal adenoma (CRC/ACRA) is provided. The method for detecting CRC/ACRA using the group of diagnostic biomarkers is also provided. For example, the method is a non-invasive approach that may utilize serum samples for detecting colorectal cancer. Moreover, the method for detecting colorectal cancer may detect colorectal cancer of different stages (e.g., pre-cancer stage, early stage, middle stage, late stage). A kit for detecting CRC/ACRA is also provided.

### CROSS-REFERENCE TO RELATED APPLICATION

The present application is a Continuation of International Application No. PCT/CN2022/130380, filed on Nov. 7, 2022, the contents of which are hereby incorporated by reference.

### TECHNICAL FIELD

The present disclosure generally relates to detection of colorectal abnormality, and in particular, to biomarkers for detecting colorectal cancer or colorectal adenoma and methods thereof.

### BACKGROUND

Colorectal cancer (CRC) usually refers to a cancer developed from the colon or rectum (parts of the large intestine). CRC has become a growing clinical challenge worldwide, while early diagnosis is recognized as an effective way to improve the survival rate for CRC patients. Colorectal adenoma (CRA) usually refers to a benign tumor located in colorectal tissue. CRA may progress into advanced colorectal adenoma (ACRA), which has a size greater than a preset limit (e.g., 1 cm), and/or presents villous architecture or high-grade dysplasia. The ACRA tends to lead to CRC. Thus, the diagnosis of ACRA or CRC is important in evaluating the colorectal health of a patient.

Several approaches have been adopted clinically for the diagnosis of CRC and/or ACRA, including non-invasive approaches and invasive approaches. For example, the non-invasive approaches include a fecal occult blood test (FOBT), using tumor biomarkers such as carcinoembryonic antigen (CEA)), etc. As another example, the invasive approaches include using a colonoscope to determine whether there are visible tumor lesions or adenomatous polyps, a biopsy test, etc. While the invasive approaches remain the gold standard for CRC/ACRA diagnosis, they often cause discomfort, pain, or even tissue damage to the patient. As a result, the non-invasive approaches are sometimes preferred. Therefore, it is desirable to find new biomarkers that can bring about non-invasive methods for the detection of CRC or ACRA.

### SUMMARY

According to an aspect of the present disclosure, a system for detecting colorectal cancer or advanced colorectal adenoma (CRC/ACRA) in a subject. The system may include at least one storage device including a set of instructions; and at least one processor in communication with the at least one storage device. When executing the set of instructions, the at least one processor is directed to perform operations including: (a) obtaining, from a quantitative measurement device, quantified abundance of one or more target metabolites in a panel of a plurality of metabolites in a sample from the subject, wherein the plurality of metabolites include the metabolites of Table 1; (b) determining a sample score by processing the quantified abundance of each of the one or more target metabolites using a prediction model; and (c) estimating whether the subject has CRC/ACRA by comparing the sample score to a cut-off score.

According to another aspect of the present disclosure, a system for detecting colorectal cancer CRC in a subject is provided. The system may include at least one storage device including a set of instructions; and at least one processor in communication with the at least one storage device. When executing the set of instructions, the at least one processor is directed to perform operations including: (a) obtaining, from a quantitative measurement device, quantified abundance of one or more target metabolites in a panel of a plurality of metabolites in a sample from the subject, wherein the plurality of metabolites include the metabolites of Table 1; (b) determining a sample score by processing the quantified abundance of each of the one or more target metabolites using a prediction model; and (c) estimating whether the subject has CRC by comparing the sample score to a cut-off score.

According to yet another aspect of the present disclosure, a system for detecting CRC in a subject is provided. The system may include at least one storage device including a set of instructions; and at least one processor in communication with the at least one storage device. When executing the set of instructions, the at least one processor is directed to perform operations including: (a) obtaining, from a quantitative measurement device, quantified abundance of one or more target metabolites in a panel of a plurality of metabolites in a sample from the subject, wherein the plurality of metabolites include the metabolites of Table 1; (b) determining a sample score by processing the quantified abundance of each of the one or more target metabolites using a prediction model; and (c) estimating whether the subject has CRC by comparing the sample score to a cut-off score.

According to yet another aspect of the present disclosure, a system for detecting early-stage CRC in a subject is provided. The system may include at least one storage device including a set of instructions; and at least one processor in communication with the at least one storage device. When executing the set of instructions, the at least one processor is directed to perform operations including: (a) obtaining, from a quantitative measurement device, quantified abundance of one or more target metabolites in a panel of a plurality of metabolites in a sample from the subject, wherein the plurality of metabolites include the metabolites of Table 1; (b) determining a sample score by processing the quantified abundance of each of the one or more target metabolites using a prediction model; and (c) estimating whether the subject has early-stage CRC by comparing the sample score to a cut-off score.

According to yet another aspect of the present disclosure, a system for detecting advanced CRC in a subject is provided. The system may include at least one storage device including a set of instructions; and at least one processor in communication with the at least one storage device. When executing the set of instructions, the at least one processor is directed to perform operations including: (a) obtaining, from a quantitative measurement device, quantified abundance of one or more target metabolites in a panel of a plurality of metabolites in a sample from the subject, wherein the plurality of metabolites include the metabolites of Table 1; (b) determining a sample score by processing the quantified abundance of each of the one or more target metabolites using a prediction model; and (c) estimating whether the subject has advanced CRC by comparing the sample score to a cut-off score.

According to still another aspect of the present disclosure, a system for detecting ACRA in a subject is provided. The system may include at least one storage device including a set of instructions; and at least one processor in communication with the at least one storage device. When executing the set of instructions, the at least one processor is directed to perform operations including: (a) obtaining, from a quantitative measurement device, quantified abundance of one or more target metabolites in a panel of a plurality of metabolites in a sample from the subject, wherein the plurality of metabolites include the metabolites of Table 1; (b) determining a sample score by processing the quantified abundance of each of the one or more target metabolites using a prediction model; and (c) estimating whether the subject has ACRA by comparing the sample score to a cut-off score.

According to yet another aspect of the present disclosure, a method of detecting colorectal cancer or advanced colorectal adenoma (CRC/ACRA) in a subject is provided. The method may include (a) obtaining, from a quantitative measurement device, quantified abundance of one or more target metabolites in a panel of a plurality of metabolites in a sample from the subject, wherein the plurality of metabolites include the metabolites of Table 1; (b) determining a sample score by processing the quantified abundance of each of the one or more target metabolites using a prediction model; (c) determining whether the subject has CRC/ACRA by at least comparing the sample score to a cut-off score. In some embodiments, the method may further include treating the subject. For example, the method may include steps (a)-(c) and further include step (d): in response to determining that the subject has CRC/ACRA, applying a treatment to the subject, wherein the treatment includes colectomy, ostomy, radiotherapy, pharmacotherapy, or a surgery for removing the ACRA or a tumor in the subject.

According to still another aspect of the present disclosure, a use of one or more target metabolites for preparing a kit for detecting colorectal cancer or advanced colorectal adenoma (CRC/ACRA) in a subject is provided. The one or more target metabolites may include at least one, two, three, four, five, eight, ten, fifteen, twenty, thirty, or forty-three of the metabolites in Table 1.

According to yet another aspect of the present disclosure, a use of one or more target metabolites in generating a trained machine-learning model for estimating whether the subject has CRC/ACRA is provided. The one or more target metabolites include at least one, two, three, four, five, eight, ten, fifteen, twenty, or twenty-six thirty, or forty-three of the metabolites in Table 1.

A kit for detecting colorectal cancer or advanced colorectal adenoma (CRC/ACRA) in a subject is provided. The kit may include one or more target metabolites in a panel of a plurality of metabolites, wherein the plurality of metabolites include the metabolites of Table 1.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a schematic diagram illustrating an exemplary system for detecting CRC/ACRA in a subject according to some embodiments of the present disclosure;

FIG. 2 is a block diagram illustrating an exemplary processing device according to some embodiments of the present disclosure;

FIG. 3A shows the performance of the prediction model for discriminating negative and positive group individuals in the training cohort;

FIG. 3B is a Principal Component Analysis (PCA) plot of the prediction model for discriminating negative and positive group individuals in the training cohort;

FIG. 4A shows the performance of the prediction model for discriminating negative and positive group individuals in the testing cohort;

FIG. 4B is a PCA plot of the prediction model for discriminating negative and positive group individuals in the testing cohort;

FIG. 5A shows the performance of the prediction model for discriminating negative subjects (including normal subjects, subjects having polyps, and subjects having low-risk colorectal adenomas) and positive subjects (including subjects having ACRA and subjects having early-stage CRC) in the testing cohort;

FIG. 5B shows the performance of the prediction model for discriminating negative subjects (including normal subjects, subjects having polyps, and subjects having low-risk colorectal adenomas) and positive subjects (including subjects having early-stage CRC and subjects having middle/late-stage CRC) in the testing cohort;

FIG. 5C shows the performance of the prediction model for discriminating negative subjects (including normal subjects, subjects having polyps, and subjects having low-risk colorectal adenomas) and positive subjects (including subjects having ACRA) in the testing cohort; and

FIG. 5D shows the performance of the prediction model for discriminating negative subjects (including normal subjects, subjects having polyps, and subjects having low-risk colorectal adenomas) and positive subjects (including subjects having early-stage CRC) in the testing cohort.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” and/or “including” when used in this specification, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawing(s), all of which form a part of this specification. It is to be expressly understood, however, that the drawing(s) is for the purpose of illustration and description only and are not intended to limit the scope of the present disclosure. It is understood that the drawings are not to scale.

The present disclosure provides a group of diagnostic biomarkers usable for detecting colorectal cancer or advanced colorectal adenoma (CRC/ACRA) in a subject. A method for detecting CRC/ACRA using the group of diagnostic biomarkers is also provided. For example, the method provided by the present disclosure is a non-invasive approach that utilizes body fluid samples (e.g., blood serum samples) for detecting CRC/ACRA. Moreover, the method for detecting CRC/ACRA may be able to detect different stages of CRC (e.g., pre-cancer stage, early stage, middle stage, late stage). ACRA tends to progress into CRC, and thus ACRA may be considered as a pre-cancer stage of CRC. The detection of early-stage or pre-cancer stage CRC in a subject may effectively improve the survival rate for CRC patients. As compared with conventional methods for detecting colorectal cancer (e.g., a method using a colonoscope or a biopsy test), the methods for detecting colorectal cancer provided by the present disclosure are non-invasive and are capable of effectively distinguishing subjects having CRC/ACRA from normal subjects.

As used herein, the term “subject” of the present disclosure refers to any human or non-human animal. Exemplary non-human animals may include Mammalia (such as chimpanzees and other apes and monkey species), farm animals (such as cattle, sheep, pigs, goats, and horses), domestic mammals (such as dogs and cats), laboratory animals (such as mice, rats, and guinea pigs), or the like. In some embodiments, the subject is a human. The term “normal subject” refers to a subject who is not suffering from CRC or ACRA. For instance, a normal subject may have a low-risk CRA (e.g., 1 or 2 adenoma(s), ≤10 mm in size, no presence of villous architecture or high-grade dysplasia) or colorectal polyps (hyperplastic polyps or inflammatory polyps). Alternatively, the normal subject may have no CRAs or colorectal polyps.

According to an aspect of the present disclosure, a group of diagnostic biomarkers usable for diagnosis of colorectal cancer (CRC) or ACRA are provided. The term “CRC/ACRA” in the present disclosure refers to either CRC or ACRA, being distinct from normal conditions.

In some embodiments, the group of diagnostic biomarkers may include one or more target metabolites correlated with CRC/ACRA. The one or more target metabolites may be serum metabolites that exhibit significant differentiation between a positive group of subjects (CRC and ACRA individuals) and a negative group of subjects (having normal conditions, colorectal polyps, or low-risk colorectal adenomas). Alternatively, the one or more target metabolites may be selected from a plurality of candidate metabolites based on the performance of machine-learning models trained using one or more of the plurality of candidate metabolites. More details regarding the determination of the one or more target metabolites may be found elsewhere in the present disclosure, e.g., Example 1.

In some embodiments, the abundance of the metabolite(s) in a sample obtained from a normal subject may be different from the abundance of the metabolite(s) in a sample obtained from a subject that has CRC/ACRA. As used herein, the term “abundance” refers to the quantity or amount of a substance in a certain sample. The sample may be a solid sample, a fluid sample, a gas sample, or the like, or any combination thereof. The solid sample may include, for example, feces, earwax, etc. The fluid sample may include the body fluid of the subject, such as blood, serum, saliva, urine, sweat, or the like, or any combination thereof. The gas sample may include flatus, breath, etc. Merely by way of example, the one or more target metabolites may be present in the serum and may be referred to as “serum metabolites”.

In some embodiments, to measure the abundance of a metabolite, the concentration or amount of the metabolite in a fluid sample (e.g., serum), a solid sample, or a gas sample (e.g., flatus) may be measured. The abundance of each of the one or more target metabolites may be quantified by a quantitative measurement device using a relative quantification approach or an absolute quantification approach. For example, the abundance of a metabolite may be a relative abundance determined based on a normalized value or a relative value with respect to a control. In some embodiments, the control may be the precise concentration or amount of a set of chemicals that are artificially added into a subject, such as spike-in control. Alternatively, the control may be the concentration or amount of the same metabolite of a sample obtained from a pool of subjects who do not have CRC/ACRA and is considered physically healthy. Alternatively, the abundance of the metabolite may be an absolute abundance that directly reflects the level of the metabolite in the subject. In some embodiments, the abundance of the metabolite may be obtained by mass spectrometry, chromatography (e.g., HPLC), and any other appropriate techniques.

Table 1 shows an exemplary group of metabolites that can be used for the diagnosis of CRC/ACRA. Each of the metabolites, which are biomarkers, has shown a strong and reliable correlation with the presence of CRC/ACRA. In some embodiments, the group of diagnostic biomarkers provided by the present disclosure may include one or more target metabolites of Table 1. In some embodiments, the group of diagnostic biomarkers may include at least one of the metabolites of Table 1. In some embodiments, the group of diagnostic biomarkers may include at least two of the metabolites of Table 1. In some embodiments, the group of diagnostic biomarkers may include at least three of the metabolites of Table 1. In some embodiments, the group of diagnostic biomarkers may include at least four of the metabolites of Table 1. In some embodiments, the group of diagnostic biomarkers may include at least five of the metabolites of Table 1. In some embodiments, the group of diagnostic biomarkers may include at least 6, 7, 8, 9, 10, 15, 20, 25, 30, 35, 40, or 43 of the metabolites of Table 1. As another example, the group of diagnostic biomarkers may include all of the metabolites of Table 1.

TABLE 1

| Meta | | MASS | Delta | Meta | | MASS | Delta |
|---|---|---|---|---|---|---|---|
| ID | Compound | (+/−) | (ppm) | ID | Compound | (+/-) | (ppm) |
| BN001 | 5_HETE | 319.228(−) | 0 | C135 | C8H10N4O2 | 195.087(+) | 2 |
| BN002 | 15_HETE | 319.228(−) | 0 | C145 | C23H46NO6P | 464.314(+) | 2 |
| BN003 | 8_HETE | 319.228(−) | 0 | C150 | C27H48NO7P | 530.324(+) | 1 |
| BN005 | 11_HETE | 319.228(−) | 0 | DS09 | TCA | 514.693(−) | 0 |
| | | | | | (Taurocholic | | |
| | | | | | Acid) | | |
| BN006 | 12_HETE | 319.228(−) | 0 | DS11 | 5β-CAA- | 391.300(−) | 0 |
| | | | | | 3β,12α-2K | | |
| BN015 | Sphingosine- | 350.210(−) | 0 | X006 | C13H16N2O4 | 263.104(−) | 1 |
| | 1-phosphate | | | | | | |
| | (d16:1) | | | | | | |
| BN016 | Octadecanedioic | 313.239(−) | 0 | X043 | C20H38O11 | 453.232(−) | 5 |
| | acid | | | | | | |
| BN017 | Epitestosterone | 367.300(−) | 0 | X082 | C19H26N6O | 353.212(−) | 7 |
| | Sulfate | | | | | | |
| BN018 | (9E,11E)-13- | 311.100(−) | 0 | X166 | C16H34NO5P | 352.224(+) | 2 |
| | Hydroperoxy-9,11- | | | | | | |
| | octadecadienoic | | | | | | |
| | acid | | | | | | |
| BN029 | Azelaic acid | 187.098(−) | 0 | X188 | C28H48NO7P | 542.324(+) | 0 |
| BN030 | Arachidonic | 303.233(−) | 0 | X278 | C11H18N2O7 | 289.106(−) | 6 |
| | acid | | | | | | |
| BN035 | 9(S),10(S), | 329.234(−) | 0 | X280 | C29H46O | 447.312(−) | 19 |
| | 13(S)_Trihydroxy_{—} | | | | | | |
| | 11(E)_Octadecenoic | | | | | | |
| | Acid | | | | | | |
| BP001 | 3- | 190.086(+) | 0 | X286 | C29H49NO5 | 512.336(−) | 1 |
| | Indolepropionic | | | | | | |
| | acid | | | | | | |
| BP010 | trans-2- | 398.326(+) | 0 | X289 | C31H41NO4 | 536.299(−) | 5 |
| | Hexadecenoyl- | | | | | | |
| | L-carnitine | | | | | | |
| BN012 | 14(S)-HDHA | 343.228(−) | 0 | X293 | C11H11NO3 | 204.067(−) | 2 |
| BN032 | Chenodeoxy | 471.235(−) | 0 | X407 | C17H17NO5 | 314.103(−) | 1 |
| | cholic Acid- | | | | | | |
| | 3-Sulfate | | | | | | |
| | Sodium Salt | | | | | | |
| BP002 | S-(−)- | 177.102(+) | 0 | X415 | C20H34O6 | 369.227(−) | 3 |
| | Cotinine | | | | | | |
| BP003 | 2- | 379.284(+) | 0 | X473 | C28H50O5 | 465.358(−) | 1 |
| | Arachidonoyl | | | | | | |
| | Glycerol | | | | | | |
| BP009 | 1-Linoleoyl- | 355.284(+) | 0 | X508 | C7H2F5NO | 212.020(+) | 33 |
| | rac-glycerol | | | | | | |
| BP013 | 17-Alpha- | 303.232(+) | 0 | X513 | C20H32O2 | 305.247(+) | 2 |
| | Methyltestosterone | | | | | | |
| C002 | C13H10O5 | 245.050(−) | 17 | X531 | C52H97NO18 | 1024.676(+) | 2 |
| C037 | C7H10OS3 | 204.982(−) | 1 | \ | \ | \ | \ |

For the metabolite annotations of Meta IDs used in the present disclosure, please refer to Table 6.

In some embodiments, one or more of the metabolites shown in Table 1 can be used for detecting CRC/ACRA in the subject. For example, mass spectrometry (or other techniques) may be used to quantify the abundance of one or more target metabolites in a panel of a plurality of metabolites in a sample. The abundance of each metabolite that has been quantified can be processed and used to detect CRC/ACRA and/or facilitate the treatment of CRC/CRC in the subject. In some embodiments, any one of the metabolites in Table 1 can be quantified and used for these purposes. In some embodiments, any two, three, or four metabolites in Table 1 can be quantified and used for these purposes. In some embodiments, any five, ten, or fifteen metabolites in Table 1 can be quantified and used for these purposes. In some embodiments, all the metabolites in Table 1 can be quantified and used for these purposes.

In some embodiments, the one or more target metabolites for detecting CRC/ACRA and/or facilitating the treatment of CRC/ACRA may include at least one metabolite of Table 1 and at least one metabolite of Table 2. Each of the metabolites in Table 2 is found to be closely correlated with the presence of CRC/ACRA. In some embodiments, the one or more target metabolites may further include 1, 2, 3, 4, 5, 6, 7, 8, 9, 10, 11, 12, 13, 14, 15, 16, 17, 18, 19, 20, 21, 30, or 38 metabolites of the metabolites in Table 2. For example, the one or more target metabolites may include one metabolite in Table 1 and one metabolite in Table 2. As another example, the one or more target metabolites may include one metabolite in Table A and two metabolites in Table B. As yet another example, the one or more target metabolites may include two metabolites in Table 1 and one metabolite in Table 2. See, e.g., Example 2. Similarly, any combinations of one or more metabolites in Table 1 and one or more metabolites in Table 2 may be used to achieve the same purposes. In some embodiments, one or more of the metabolites in Table 2 may be used, independently from the metabolites listed in Table 1, for detecting and/or facilitating the treatment of CRC/ACRA in the subject.

TABLE 2

| Meta | | MASS | Delta | Meta | | MASS | Delta |
|---|---|---|---|---|---|---|---|
| ID | Compound | (+/−) | (ppm) | ID | Compound | (+/-) | (ppm) |
| BN010 | 9(10)_{—} | 313.239(−) | 0 | C118 | C22H34O2 | 331.262(+) | 1 |
| | DiHOME | | | | | | |
| BN011 | 12(13)_{—} | 313.239(−) | 0 | C119 | C21H36O3 | 337.273(+) | 2 |
| | DiHOME | | | | | | |
| BN031 | 3- | 173.119(−) | 0 | C138 | C16H31NO4 | 302.233(+) | 2 |
| | Hydroxynonanoic | | | | | | |
| | acid | | | | | | |
| BN034 | 1-Palmitoyl- | 452.279(−) | 0 | DS01 | CA (Cholic | 407.281(−) | 0 |
| | 2-hydroxy- | | | | Acid) | | |
| | sn-glycero- | | | | | | |
| | 3-PE | | | | | | |
| BP006 | Myristoyl-L- | 372.311(+) | 0 | DS02 | CDCA | 391.286(−) | 0 |
| | carnitine | | | | (Chenodeoxy | | |
| | | | | | cholic Acid) | | |
| BN019 | 9(S),10(S), | 329.234(−) | 0 | DS03 | DCA | 391.286(−) | 0 |
| | 13(S)- | | | | (Deoxycholic | | |
| | Trihydroxy- | | | | Acid) | | |
| | 10(E)- | | | | | | |
| | Octadecenoic | | | | | | |
| | Acid | | | | | | |
| BN020 | 3a-Hydroxy- | 389.270(−) | 0 | DS04 | GCA | 464.302(−) | 0 |
| | 6-OXO-5α- | | | | (Glycocholic | | |
| | Cholan-24- | | | | Acid | | |
| | OIC Acid | | | | Hydrate) | | |
| BN021 | 3- | 405.265(−) | 0 | DS05 | GCDCA | 448.307(−) | 0 |
| | Dehydrocholic | | | | (Glycocheno | | |
| | Acid | | | | deoxycholic | | |
| | | | | | Acid) | | |
| BN022 | 5α-Cholanic | 405.265(−) | 0 | DS06 | GDCA | 448.307(−) | 0 |
| | Acid-3α,7β- | | | | (Glycodeoxy | | |
| | Diol-6-One | | | | cholic Acid) | | |
| BN023 | Eicosapentaenoic | 301.218(−) | 0 | DS07 | GLCA | 432.312(−) | 0 |
| | Acid | | | | (Glycolithoch | | |
| | | | | | olic Acid) | | |
| BN025 | Hydrocortisone | 361.202(−) | 0 | DS08 | GUDCA | 448.307(−) | 0 |
| | | | | | (Glycoursode | | |
| | | | | | oxycholic | | |
| | | | | | Acid) | | |
| BN027 | 1-Stearoyl- | 480.310(−) | 0 | DS10 | UDCA | 391.286(−) | 0 |
| | 2-Hydroxy- | | | | (Ursodeoxycholic | | |
| | sn-Glycero- | | | | Acid) | | |
| | 3- | | | | | | |
| | Phosphoethanolamine | | | | | | |
| BN028 | Pimelic acid | 159.067(−) | 0 | X042 | C21H44NO7 | 452.278(−) | 1 |
| BP007 | trans-2- | 286.201(+) | 0 | X066 | C29H56NO9P | 592.362(−) | 0 |
| | octenoyl-l- | | | | | | |
| | carnitine | | | | | | |
| BP008 | 2- | 246.170(+) | 0 | X401 | C12H17NO3 | 222.114(−) | 2 |
| | Methylbutyryl-L- | | | | | | |
| | carnitine | | | | | | |
| BP011 | Decanoyl-L- | 316.248(+) | 0 | X459 | C43H73N3O16P2 | 930.431 (−) | 3 |
| | carnitine | | | | | | |
| BP012 | (±)- | 261.193(+) | 0 | X511 | C17H26O2 | 263.200(+) | 2 |
| | Hexanoyl | | | | | | |
| | carnitine | | | | | | |
| | chloride | | | | | | |
| C004 | C10H12N4O5 | 267.073(−) | 2 | X544 | C26H29NO9 | 500.170(+) | 43 |
| BN004 | 9_HETE | 319.228(−) | 0 | C008 | C19H36O4 | 327.256(−) | 5 |

In some embodiments, the one or more target metabolites provided by the present disclosure may include at least one metabolite of Table 3 Each of the metabolites in Table 3 is found to be correlated with the presence of CRC/ACRA. In some embodiments, one or more of the metabolites in Table 3 may be used, in addition to the one or more metabolites listed in Table 1 and/or one or more metabolites listed in Table 2, for detecting CRC/ACRA and/or facilitating the treatment of CRC/ACRA in the subject. In some embodiments, the abundance of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 120, 140, or 166 metabolites of the metabolites in Table 3 may be quantified for the same purposes.

TABLE 3

| Meta | | MASS | Delta |
|---|---|---|---|
| ID | Compound | (+/−) | (ppm) |
| C001 | C12H22O4 | 229.144(−) | 2 |
| C005 | C16H32O3 | 271.228(−) | 2 |
| C009 | C22H20N4O | 355.158(−) | 4 |
| C011 | C16H30O10 | 381.174(−) | 7 |
| C012 | C19H21N5O3S | 398.132(−) | 7 |
| C014 | C21H24FN3O2S | 400.148(−) | 5 |
| C016 | C24H23F3N2O2 | 427.163(−) | 2 |
| C017 | C27H52O4 | 439.379(−) | 0 |
| C019 | C27H44O5 | 447.312(−) | 1 |
| C020 | C25H38O8 | 465.249(−) | 1 |
| C022 | C20H38O12 | 469.227(−) | 4 |
| C023 | C21H34N4O8 | 469.227(−) | 7 |
| C027 | C27H48O4 | 481.354(−) | 1 |
| C028 | C24H45O9P | 507.273(−) | 0 |
| C029 | C29H33N7O2 | 510.253(−) | 18 |
| C030 | C27H48NO7P | 528.310(−) | 1 |
| C035 | C33H45N5O5 | 590.346(−) | 19 |
| C036 | C31H57N5O9 | 642.396(−) | 19 |
| C038 | C18H32O2 | 279.233(−) | 0 |
| C040 | C22H34O2 | 329.248(−) | 0 |
| C041 | C26H44O9 | 499.288(−) | 7 |
| C102 | C7H8N4O2 | 181.072(+) | 1 |
| C108 | C15H26O4 | 271.190(+) | 1 |
| C110 | C15H27NO4 | 286.201(+) | 1 |
| C112 | C17H18N2O4 | 315.134(+) | 0 |
| C115 | C16H28O6 | 317.195(+) | 3 |
| C121 | C20H39NO4 | 358.295(+) | 1 |
| C122 | C24H37NO2 | 372.300(+) | 28 |
| C124 | C23H43NO4 | 398.325(+) | 4 |
| C127 | C25H47NO4 | 426.357(+) | 2 |
| C129 | C25H47NO5 | 442.352(+) | 2 |
| C130 | C21H42NO7P | 452.277(+) | 0 |
| C131 | C22H46NO7P | 468.308(+) | 1 |
| C136 | C19H26O2 | 287.204(+) | 14 |
| C144 | C24H36O2 | 357.280(+) | 3 |
| C146 | C23H48NO7P | 482.324(+) | 0 |
| C148 | C25H50NO7P | 508.340(+) | 1 |
| X004 | C12H16O5 | 239.092(−) | 2 |
| X010 | C17H34O | 299.259(−) | 1 |
| X012 | C17H18N2O4 | 313.119(−) | 1 |
| X013 | C18H34O4 | 313.238(−) | 1 |
| X016 | C20H32O3 | 319.228(−) | 0 |
| X023 | C19H28O5S | 367.158(−) | 1 |
| X024 | C19H30O5S | 369.174(−) | 0 |
| X032 | C25H24O5 | 403.158(−) | 7 |
| X036 | C23H27FN4O3 | 425.201(−) | 4 |
| X055 | C23H48NO7P | 526.315(−) | 0 |
| X068 | C32H51NO11 | 624.339(−) | 0 |
| X070 | C34H66NO8P | 646.427(−) | 28 |
| X133 | C7H10O3 | 143.07(+) | 2 |
| X139 | C11H20O4 | 217.143(+) | 2 |
| X141 | C11H22N2O3 | 231.174(+) | 16 |
| X144 | C17H33NO4 | 257.174(+) | 3 |
| X152 | C18H25N3O | 300.216(+) | 30 |
| X155 | C20H30O2 | 303.231(+) | 3 |
| X183 | C13H17N3O | 490.300(+) | 5 |
| X196 | C21H24N4O2 | 347.196(+) | 25 |
| X281 | C50H89O11P | 447.312(−) | 27 |
| X285 | C28H47NO6 | 512.336(−) | 5 |
| X292 | C7H8O4S | 187.007(−) | 0 |
| X314 | C32H31O14 | 637.157(−) | 0 |
| X402 | C10H18O2 | 229.144(−) | 2 |
| X403 | C13H12N4O | 239.092(−) | 8 |
| X405 | C17H3O | 279.233(−) | 0 |
| X409 | C20H34O | 335.259(−) | 0 |
| X412 | C19H22N4O3 | 353.164(−) | 6 |
| X419 | C12H22S2 | 229.108(−) | 4 |
| X426 | C10H18O4 | 201.113(−) | 1 |
| X429 | C22H2201O | 445.114(−) | 0 |
| X431 | C23H34N4O5 | 445.244(−) | 4 |
| X432 | C10H16O3 | 183.103(−) | 2 |
| X433 | C11H14N4 | 201.113(−) | 8 |
| X434 | C11H24N6O3 | 287.186(−) | 8 |
| X435 | C11H22O3 | 201.150(−) | 2 |
| X436 | C20H34O8 | 401.218(−) | 0 |
| X437 | C16H30O5 | 301.202(−) | 0 |
| X448 | C18H34O5 | 329.233(−) | 1 |
| X449 | C39H61O8P | 687.398(−) | 7 |
| X451 | C41H65O8P | 715.429(−) | 8 |
| X454 | C35H61O10P | 671.402(−) | 13 |
| X455 | C37H67O9P | 685.419(−) | 38 |
| X456 | C38H69O9P | 699.434(−) | 38 |
| X457 | C28H48O5 | 463.343(−) | 0 |
| X458 | C18H36O3 | 299.259(−) | 1 |
| X463 | C9H14O | 183.103(−) | 2 |
| X470 | C37H68NO9P | 700.436(−) | 28 |
| X471 | C39H71O9P | 713.450(−) | 37 |
| X509 | C13H20O3 | 225.148(+) | 2 |
| X512 | C18H30O | 263.236(+) | 4 |
| X515 | C19H18N4O2 | 335.151(+) | 2 |
| X517 | C18H24NO4P | 350.153(+) | 4 |
| X519 | C13H14O11 | 364.084(+) | 9 |
| X523 | C26H48O9 | 505.336(+) | 2 |
| X525 | C34H58O6 | 563.427(+) | 6 |
| X535 | C13H21NO2 | 224.165(+) | 2 |
| X536 | C12H16O2 | 193.122(+) | 2 |
| X540 | C25H52NO4P | 484.348(+) | 10 |
| X541 | C14H22O4 | 255.159(+) | 0 |
| X542 | C13H18O2 | 207.138(+) | 0 |
| X543 | C24H30N8O | 447.259(+) | 6 |
| X546 | C14H22O3 | 271.190(+) | 1 |
| X547 | C15H28O5 | 289.201(+) | 0 |
| X549 | C12H26N2O7 | 311.183(+) | 6 |
| X550 | C33H45N5O5 | 630.306(+) | 1 |
| X555 | C20H32O7 | 385.222(+) | 0 |
| X556 | C27H33NO3 | 420.259(+) | 14 |
| X557 | C20H29N3O5S2 | 456.144(+) | 40 |
| X558 | C27H38O2 | 395.300(+) | 14 |
| X559 | C24H40O6S | 457.279(+) | 38 |
| X560 | C36H60O6 | 589.435(+) | 19 |
| X561 | C37H53O4 | 544.406(+) | 25 |
| X563 | C18H35NO4 | 271.190(+) | 1 |
| X565 | C35H64O9 | 629.447(+) | 24 |
| X566 | C32H54O5 | 602.448(+) | 7 |
| X567 | C29H54O7 | 514.395(+) | 28 |
| X569 | C34H61NO6 | 602.447(+) | 13 |
| X570 | C18H26O | 558.421(+) | 17 |
| X571 | C29H52O6 | 514.395(+) | 30 |
| X572 | C27H51NO3S | 470.369(+) | 6 |
| X573 | C25H40O4 | 405.301(+) | 3 |
| X574 | C40H78NO9P | 748.542(+) | 9 |
| X575 | C42H82NO10P | 792.568(+) | 9 |
| X576 | C43H65NO4 | 660.489(+) | 15 |
| X577 | C26H44O8 | 485.311(+) | 0 |
| X578 | C38H74NO8P | 704.516(+) | 9 |
| X579 | C37H58O6 | 599.437(+) | 11 |
| X580 | C38H64O6 | 617.467(+) | 17 |
| X581 | C35H66NO8P | 660.489(+) | 44 |
| X582 | C28H46N2O6 | 507.336(+) | 14 |
| X583 | C36H61NO4 | 572.437(+) | 53 |
| X584 | C29H51O5P | 511.384(+) | 57 |
| X585 | C16H26O2 | 528.411(+) | 2 |
| X586 | C38H65NO5 | 616.463(+) | 50 |
| X587 | C39H76NO8P | 718.531(+) | 10 |
| X588 | C29H4803 | 528.411(+) | 8 |
| X589 | C39H73O8P | 701.505(+) | 9 |
| X591 | C37H72NO7P | 674.505(+) | 10 |
| X592 | C38H63NO6 | 630.479(+) | 10 |
| X593 | C37H63NO4 | 586.452(+) | 53 |
| X595 | C42H82NO9P | 776.573(+) | 9 |
| X596 | C28H56NO7P | 550.390(+) | 6 |
| X597 | C40H78NO8P | 732.547(+) | 9 |
| X598 | C31H46O2 | 514.377(+) | 22 |
| X599 | C38H74NO7P | 688.521(+) | 10 |
| X600 | C37H67O8P | 671.494(+) | 44 |
| X601 | C36H69NO8 | 644.494(+) | 24 |
| X602 | C34H54O5 | 543.398(+) | 12 |
| X603 | C31H52O6 | 521.384(+) | 1 |
| X605 | C50H88NO9P | 878.640(+) | 15 |
| X606 | C33H57O10P | 645.385(+) | 14 |
| X608 | C38H65NO4 | 600.468(+) | 51 |
| X609 | C41H80NO8P | 746.562(+) | 10 |
| X614 | C35H61O10P | 673.416(+) | 13 |
| X616 | C17H37N7O4 | 404.301(+) | 7 |
| X617 | C36H63O10P | 687.431(+) | 11 |
| X618 | C32H48O5 | 513.342(+) | 30 |
| X619 | C37H65O10P | 701.447(+) | 12 |
| X620 | C38H72NO9P | 718.474(+) | 39 |
| X621 | C26H40O3 | 401.290(+) | 37 |
| X624 | C27H40N4O4 | 485.311(+) | 3 |
| X628 | C31H54O9 | 571.384(+) | 0 |
| X629 | C38H67O10P | 715.463(+) | 12 |
| X633 | C23H40O5 | 397.295(+) | 0 |
| X634 | C25H38N2O3 | 415.305(+) | 23 |
| X635 | C27H45NO3 | 432.332(+) | 35 |
| X639 | C35H58O8 | 690.463(+) | 8 |

In some embodiments, the one or more target metabolites provided by the present disclosure may include at least one metabolite of Table 4. Each of the metabolites in Table 4 is found to be correlated with the presence of CRC/ACRA. In some embodiments, one or more of the metabolites in Table 4 may be used, in addition to the one or more metabolites listed in Table 1 and/or one or more metabolites listed in Table 2, for detecting CRC/ACRA and/or facilitating the treatment of CRC/ACRA in the subject. As another example, one or more of the metabolites in Table 4 may be used, in addition to the one or more metabolites listed in Table 1, one or more metabolites listed in Table 2, and one or more metabolites listed in Table 3 for the same purposes. In some embodiments, the abundance of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, or 116 metabolites of the metabolites in Table 4 may be quantified.

TABLE 4

| Meta | | MASS | Delta | Meta | | MASS | Delta |
|---|---|---|---|---|---|---|---|
| ID | Compound | (+/−) | (ppm) | ID | Compound | (+/−) | (ppm) |
| C006 | C18H32O3 | 295.229(−) | 2 | X452 | C37H62O13 | 713.414(−) | 3 |
| C010 | C22H18FN3O2 | 374.132(−) | 3 | X453 | C15H17N3 | 716.431 (−) | 16 |
| C013 | C21H36O5S | 399.221(−) | 0 | X460 | C18H14 | 229.108(−) | 25 |
| C015 | C24H40O5 | 407.280(−) | 0 | X461 | C14H7F6N3O | 346.035(−) | 20 |
| C021 | C29H43NO2S | 468.308(−) | 30 | X462 | C18H15N3O3S | 390.025(−) | 18 |
| C024 | C24H40O7S | 471.242(−) | 0 | X464 | C19H32O9 | 403.197(−) | 1 |
| C025 | C25H43N3O6 | 480.308(−) | 0 | X465 | C37H54O11 | 673.382(−) | 34 |
| C026 | C25H43N3O6 | 480.310(−) | 4 | X466 | C42H56O8 | 687.396(−) | 8 |
| C031 | C34H43N3O3 | 540.331(−) | 14 | X467 | C36H63O11P | 701.413(−) | 14 |
| C032 | C33H35N5O5 | 580.235(−) | 37 | X468 | C37H65O11P | 715.429(−) | 14 |
| C033 | C33H34N4O6 | 581.241(−) | 1 | X469 | C43H69O8P | 743.459(−) | 9 |
| C034 | C37H47NO5 | 584.357(−) | 32 | X472 | C43H70NO8P | 758.481 (−) | 6 |
| C042 | C31H44O6 | 511.302(−) | 9 | X474 | C41H75O10P | 757.477(−) | 34 |
| C043 | C24H49O9P | 511.302(−) | 4 | X475 | C39H69O10P | 727.464(−) | 12 |
| C105 | C12H23NO4 | 246.170(+) | 0 | X476 | C45H73O8P | 771.493(−) | 5 |
| C113 | C15H17F3N2O2 | 315.134(+) | 9 | X477 | C45H76NO8P | 788.519(−) | 6 |
| C114 | C19H37NO6 | 317.195(+) | 3 | X478 | C41H75N2O10P | 785.508(−) | 1 |
| C116 | C18H35NO4 | 330.263(+) | 2 | X479 | C42H77N2O10P | 799.524(−) | 0 |
| C120 | C21H38O4 | 355.283(+) | 4 | X480 | C51H89O14P | 955.601 (−) | 10 |
| C128 | C25H48O5 | 428.363(+) | 23 | X516 | C21H22O4 | 339.162(+) | 9 |
| C132 | C23H21N5O5S | 480.134(+) | 1 | X518 | C21H19FN4O | 363.162(+) | 1 |
| C134 | C14H22N2O2 | 518.368(+) | 4 | X532 | C25H40N4O5 | 477.307(+) | 0 |
| C137 | C19H27NO2 | 302.215(+) | 11 | X533 | C9H12 | 121.101(+) | 1 |
| C139 | C21H40O3 | 341.306(+) | 2 | X534 | C13H22O4 | 243.159(+) | 0 |
| C140 | C23H36N2 | 341.306(+) | 31 | X537 | C12H19NO3 | 226.144(+) | 1 |
| C141 | C23H30O3 | 355.227(+) | 1 | X538 | C27H41N5O4 | 500.343(+) | 40 |
| C142 | C20H24O2 | 357.243(+) | 0 | X539 | C30H51NO6 | 544.369(+) | 15 |
| C143 | C21H31F3O | 357.243(+) | 9 | X545 | C15H24O3 | 253.180(+) | 1 |
| C147 | C25H47NO9 | 506.323(+) | 18 | X548 | C22H27N | 306.227(+) | 18 |
| C149 | C28H45NO7 | 508.340(+) | 26 | X551 | C19H32O5 | 341.232(+) | 1 |
| X011 | C18H32O4 | 311.223(−) | 1 | X552 | C14H22O3 | 239.164(+) | 1 |
| X019 | C22H32O3 | 343.228(−) | 0 | X553 | C23H36O7 | 425.253(+) | 1 |
| X051 | C32H43NO4 | 504.310(−) | 4 | X554 | C11H18O4 | 215.128(+) | 1 |
| X154 | C16H23N5O | 302.196(+) | 5 | X562 | C27H41NO6 | 476.294(+) | 14 |
| X160 | C17H33NO4 | 316.247(+) | 4 | X564 | C28H32N4O2 | 457.280(+) | 44 |
| X178 | C26H43NO5 | 432.311(+) | 1 | X568 | C26H38N4O4 | 471.296(+) | 1 |
| X408 | C20H30O3 | 317.212(−) | 1 | X590 | C20H33NO14 | 512.207(+) | 19 |
| X411 | C22H34O3 | 345.243(−) | 2 | X594 | C44H86NO10P | 820.601(+) | 6 |
| X416 | C20H25NO2S2 | 374.132(−) | 18 | X604 | C36H65NO13 | 720.453(+) | 0 |
| X421 | C10H14N5O6PS | 362.030(−) | 8 | X607 | C31H51N9O7 | 662.411(+) | 19 |
| X422 | C9H15NO2 | 168.103(−) | 0 | X610 | C38H69O10P | 717.442(+) | 37 |
| X423 | C9H18O3 | 173.118(−) | 2 | X611 | C41H68NO8P | 734.469(+) | 9 |
| X424 | C9H11NO6S | 297.983(−) | 12 | X612 | C35H63O9P | 659.400(+) | 43 |
| X425 | C16H12CI2FN5 | 362.030(−) | 22 | X613 | C34H62NO10P | 676.426(+) | 11 |
| X427 | C25H26O4 | 389.181(−) | 13 | X615 | C26H48N6O8 | 573.363(+) | 4 |
| X428 | C13H19N5O | 260.158(−) | 24 | X622 | C39H69O11P | 745.473(+) | 11 |
| X430 | C21H36O9 | 431.228(−) | 2 | X623 | C40H71O11P | 759.489(+) | 11 |
| X438 | C22H32N4O4 | 415.234(−) | 3 | X625 | C40H74NO11P | 776.515(+) | 10 |
| X439 | C23H28N8 | 415.234(−) | 6 | X626 | C44H75O9P | 779.524(+) | 2 |
| X440 | C31H40N2O3 | 487.291 (−) | 12 | X627 | C14H24O4 | 513.342(+) | 0 |
| X441 | C24H34N2O5 | 429.249(−) | 22 | X630 | C40H69O10P | 741.478(+) | 11 |
| X442 | C28H32O13 | 575.175(−) | 4 | X631 | C41H70NO8P | 812.400(+) | 4 |
| X443 | C28H31FN2 C | 429.249(−) | 33 | X632 | C14H26O2 | 227.200(+) | 2 |
| X444 | C23H40O8 | 443.265(−) | 0 | X636 | C41H73O11P | 773.505(+) | 11 |
| X445 | C33H57O11P | 659.366(−) | 14 | X637 | C42H75O11P | 787.520(+) | 10 |
| X446 | C28H42O5 | 457.280(−) | 35 | X638 | C18H32N8O4 | 425.253(+) | 21 |
| X447 | C32H28F3N5O4 | 602.203(−) | 2 | X640 | C41H73O9P | 741.478(+) | 38 |
| X450 | C32H54O12 | 629.355(−) | 1 | X641 | C9H16O2 | 157.122(+) | 2 |

In some embodiments, the one or more target metabolites may include one or more metabolite combinations shown in Table 5.

TABLE 5

| | Metabolite |
|---|---|
| No. | combination |
| 1 | BN010 + C004 |
| 2 | BN011 + DS01 |
| 3 | BN019 + C004 |
| 4 | BN020 + C004 |
| 5 | BN021 + X554 |
| 6 | BN022 + DS01 |
| 7 | BN023 + DS01 |
| 8 | BN025 + C004 |
| 9 | BN027 + C004 |
| 10 | BP007 + C004 |
| 11 | BP008 + C004 |
| 12 | BP011 + C004 |
| 13 | BP012 + DS01 |
| 14 | C004 + X019 |
| 15 | C006 + X475 |
| 16 | C008 + C008 |
| 17 | C013 + DS01 |
| 18 | C021 + X611 |
| 19 | C025 + X066 |
| 20 | C026 + X611 |
| 21 | C032 + X611 |
| 22 | C033 + X627 |
| 23 | C034 + X611 |
| 24 | C042 + C132 |
| 25 | C043 + C105 |
| 26 | C105 + C128 |
| 27 | C113 + C132 |
| 28 | C114 + DS01 |
| 29 | C116 + X154 |
| 30 | C118 + X562 |
| 31 | C119 + X594 |
| 32 | C128 + C132 |
| 33 | C132 + C149 |
| 34 | C134 + X446 |
| 35 | C137 + C137 |
| 36 | C138 + DS01 |
| 37 | C139 + X594 |
| 38 | C140 + X594 |
| 39 | C142 + X590 |
| 40 | C143 + DS02 |
| 41 | C147 + C149 |
| 42 | C149 + X066 |
| 43 | DS01 + X532 |
| 44 | DS02 + X590 |
| 45 | DS03 + DS03 |
| 46 | DS04 + DS04 |
| 47 | DS05 + X066 |
| 48 | DS06 + X594 |
| 49 | DS07 + X604 |
| 50 | DS08 + DS08 |
| 51 | DS10 + X564 |
| 52 | X011 + X011 |
| 53 | X019 + X446 |
| 54 | X051 + X066 |
| 55 | X066 + X518 |
| 56 | X154 + X154 |
| 57 | X160 + X590 |
| 58 | X401 + X544 |
| 59 | X408 + X518 |
| 60 | X411 + X562 |
| 61 | X416 + X594 |
| 62 | X421 + X450 |
| 63 | X422 + X450 |
| 64 | X423 + X464 |
| 65 | X424 + X464 |
| 66 | X425 + X446 |
| 67 | X427 + X604 |
| 68 | X428 + X532 |
| 69 | X430 + X450 |
| 70 | X438 + X446 |
| 71 | X439 + X446 |
| 72 | X440 + X450 |
| 73 | X441 + X444 |
| 74 | X442 + X594 |
| 75 | X443 + X446 |
| 76 | X444 + X464 |
| 77 | X445 + X450 |
| 78 | X446 + X465 |
| 79 | X447 + X518 |
| 80 | X450 + X465 |
| 81 | X452 + X452 |
| 82 | X453 + X518 |
| 83 | X459 + X562 |
| 84 | X460 + X460 |
| 85 | X461 + X475 |
| 86 | X462 + X532 |
| 87 | X464 + X607 |
| 88 | X465 + X607 |
| 89 | X466 + X607 |
| 90 | X467 + X475 |
| 91 | X468 + X475 |
| 92 | X469 + X475 |
| 93 | X472 + X607 |
| 94 | X474 + X475 |
| 95 | X475 + X562 |
| 96 | X476 + X607 |
| 97 | X477 + X607 |
| 98 | X478 + X607 |
| 99 | X479 + X607 |
| 100 | X480 + X607 |
| 101 | X518 + X611 |
| 102 | X532 + X627 |
| 103 | X533 + X590 |
| 104 | X534 + X594 |
| 105 | X537 + X590 |
| 106 | X538 + X590 |
| 107 | X544 + X590 |
| 108 | X545 + X548 |
| 109 | X548 + X594 |
| 110 | X551 + X562 |
| 111 | X552 + X607 |
| 112 | X554 + X607 |
| 113 | X562 + X607 |
| 114 | X564 + X607 |
| 115 | X568 + X607 |
| 116 | X590 + X615 |
| 117 | X594 + X611 |
| 118 | X604 + X607 |
| 119 | X607 + X615 |
| 120 | X610 + X613 |
| 121 | X611 + X613 |
| 122 | X612 + X613 |
| 123 | X613 + X636 |
| 124 | X615 + X615 |
| 125 | X622 + X631 |
| 126 | X623 + X623 |
| 127 | X625 + X626 |
| 128 | X626 + X637 |
| 129 | X627 + X627 |
| 130 | X630 + X637 |
| 131 | X631 + X631 |
| 132 | X632 + X632 |
| 133 | X636 + X637 |
| 134 | X637 + X637 |
| 135 | X638 + X638 |
| 136 | X640 + X640 |
| 137 | X641 + X641 |
| \ | \ |

In some embodiments, the one or more target metabolites may include the one or more metabolic combinations shown in Table 5 but exclude any metabolites shown in Table 1. Alternatively, the one or more target metabolites may include the one or more metabolic combinations shown in Table 5 and at least one metabolite selected from the metabolites in Tables 1-4.

It should be noted that one or more of the metabolites listed in Table 1-5 may have one or more isomeride forms, which are included in the scope of the group of diagnostic biomarkers provided by the present disclosure.

According to another aspect of the present disclosure, a method of detecting CRC/ACRA in a subject is provided. In some embodiments, the method may include: (a) obtaining, from a quantitative measurement device, quantified abundance of one or more target metabolites in a panel of a plurality of metabolites in a sample from the subject; (b) determining a sample score by processing the quantified abundance of each of the one or more target metabolites using a prediction model; and (c) estimating whether the subject has CRC/ACRA by comparing the sample score to a cut-off score. The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-4, and/or one or more metabolic combinations in Table 5.

In some embodiments, the abundance of the one or more components of the panel of the plurality of metabolites may be measured using mass spectrometry (MS; e.g., liquid chromatography-mass spectrometry (LC-MS), gas chromatography-mass spectrometry (GC-MS); matrix-assisted laser desorption/ionization time-of-flight mass spectrometry (MALDI-TOF MS)), ultraviolet spectrometry, High-Performance Liquid Chromatography (HPLC), or the like. In some embodiments, step b) may further include normalizing the abundance of each of the metabolites quantified in step (a), and determining the sample score by processing the normalized abundance with a prediction model.

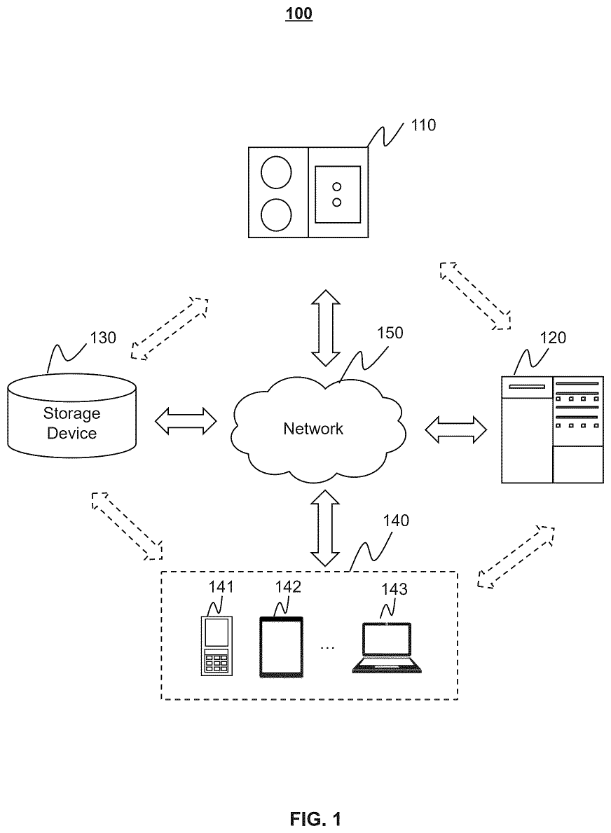

In some embodiments, the determination of the sample score may be implemented on a computing device (e.g., the processing device 120 illustrated in FIG. 1). The computing device may obtain a prediction model for determining the sample score. The abundance of each of the metabolites quantified in step a) may be inputted into the prediction model. The prediction model may process the abundance (e.g., a relative abundance or an absolute abundance) of each of the metabolites quantified in step a) and output the sample score. Merely by way of example, the abundance of each of the metabolites quantified in step a) may be quantified by measuring the concentration of each of the metabolites. In some embodiments, the measured concentration may be normalized. For instance, the measured concentration may be divided by a total concentration of all metabolites in the sample. The sample score may indicate a probability that the subject has CRC/ACRA.

In some embodiments, the prediction model may be a trained machine-learning model. For example, the prediction model may be generated using a gradient boosting decision tree (GBDT) algorithm, a decision tree algorithm, a Random Forest algorithm, a logistic regression algorithm, a support vector machine (SVM) algorithm, a Naive Bayesian algorithm, an AdaBoost algorithm, a K-a nearest neighbor (KNN) algorithm, a Markov Chains algorithm, an XGBoosting algorithm, a deep learning algorithm, a neural network, or the like, or any combination thereof, which is not limited by the present disclosure.

To obtain the prediction model, a preliminary model may be trained using a plurality of training datasets. Each of the plurality of training datasets may include a quantified abundance of a sample metabolite of a reference subject and a label indicating whether the reference subject has CRC/ACRA or is normal. The plurality of reference subjects may include a plurality of normal subjects who do not CRC/ACRA, a plurality of subjects having CRC, and a plurality of subjects having CRA. Merely by way of example, the label may be a positive label or a negative label. The positive label indicates that the reference subject has CRC/ACRA, and the negative label indicates that the reference subject is normal, or has a colorectal polyp, or has a normal colorectal adenoma (i.e., a low-risk colorectal adenoma that does not tend to lead to CRC). If a reference subject is not suffering from CRC/ACRA, the corresponding label may be designated as 0 (i.e., as a negative label). If a reference subject has CRC/ACRA, the corresponding label may be designated as 1 (i.e., as a positive sample). Accordingly, the sample score outputted by the prediction model may be a value between 0 and 1. The closer the sample score is to 1, the higher the probability that the subject has CRC/ACRA is.

In step c), the sample score is compared to a cut-off score related to the prediction model. As used herein, the term “cut-off value” refers to a dividing point on measuring scales where evaluation results are divided into different categories. In some embodiments, when the sample score is equal to or greater than the cut-off score, the computing device may determine that the subject has CRC/ACRA. The cut-off value may be determined based on the performance of the prediction model. In some embodiments, the cut-off value may be a value between 0.35-0.65. In some embodiments, the cut-off value may be a value between 0.40-0.60. In some embodiments, the cut-off value may be a value between 0.45-0.55. For example, the cut-off value may be 0.48, 0.50, 0.52, 0.541, 0.55, etc.

In some embodiments, the prediction model may be used to distinguish normal people from CRC patients in different stages. In some embodiments, the plurality of reference subjects having CRC may include a plurality of subjects having a pre-cancer stage (stage 0) CRC, or ACRA. In some embodiments, the plurality of reference subjects having CRC may include a plurality of subjects having an early stage (stage I) CRC. In some embodiments, the plurality of reference subjects having CRC may include a plurality of subjects having a middle-stage (stage II) CRC. In some embodiments, the plurality of reference subjects having CRC may include a plurality of subjects having a late stage (stage III and IV) CRC. In some embodiments, the prediction model for detecting CRC may be established in a manner similar to the prediction model for detecting CRC/ACRA as described earlier in the present disclosure.

In some embodiments, a receiver operating characteristic (ROC) curve may be used to evaluate the performance of the prediction model. The ROC curve may illustrate the diagnostic ability of the prediction model as its cut-off value is varied. The ROC curve is usually generated by plotting the sensitivity against the specificity. An area-under-the-curve (AUC) may be determined based on the ROC curve. The AUC may indicate the probability that a classifier (i.e., the prediction model) will rank a randomly chosen positive instance higher than a randomly chosen negative one.

In some embodiments, the AUC of the prediction model provided by the present disclosure is more than 0.55. In some embodiments, the AUC of the prediction model provided by the present disclosure is more than 0.70. In some embodiments, the AUC of the prediction model provided by the present disclosure is more than 0.75. In some embodiments, the AUC of the prediction model provided by the present disclosure is more than 0.8. In some embodiments, the AUC of the prediction model provided by the present disclosure is more than 0.85. In some embodiments, the AUC of the prediction model provided by the present disclosure is more than 0.9. In some embodiments, the AUC of the prediction model provided by the present disclosure is more than 0.95.

In some embodiments, the sensitivity of the prediction model for detecting CRC/ACRA is equal to or greater than 60%. In some embodiments, the sensitivity of the prediction model for detecting CRC/ACRA is equal to or greater than 70%. In some embodiments, the sensitivity of the prediction model for detecting CRC/ACRA is equal to or greater than 75%. In some embodiments, the sensitivity of the prediction model for detecting CRC/ACRA is equal to or greater than 80%. In some embodiments, the sensitivity of the prediction model for detecting CRC/ACRA is equal to or greater than 90%. In some embodiments, the sensitivity of the prediction model for detecting CRC/ACRA is equal to or greater than 95%.

In some embodiments, the specificity of the prediction model for detecting CRC/ACRA is equal to or greater than 60%. In some embodiments, the specificity of the prediction model for detecting CRC/ACRA is equal to or greater than 70%. In some embodiments, the specificity of the prediction model for detecting CRC/ACRA is equal to or greater than 75%. In some embodiments, the specificity of the prediction model for detecting CRC/ACRA is equal to or greater than 80%. In some embodiments, the specificity of the prediction model for detecting CRC/ACRA is equal to or greater than 90%. In some embodiments, the specificity of the prediction model for detecting CRC/ACRA is equal to or greater than 95%.

More descriptions regarding the performance of some exemplary prediction models for detecting CRC/ACRA may be found in the Examples section.

FIG. 1 is a schematic diagram illustrating an exemplary system for detecting CRC/ACRA in a subject according to some embodiments of the present disclosure. In some embodiments, the method for detecting CRC/ACRA in a subject may be implemented on the system 100. As illustrated, the system 100 may include a quantitative measurement device 110, a processing device 120, a storage device 130, a terminal device 140, and a network 150. The components of the system 100 may be connected in various ways. Merely by way of example, as illustrated in FIG. 1, the quantitative measurement device 110 may be connected to the processing device 120 directly as indicated by the bi-directional arrow in dotted lines linking the quantitative measurement device 110 and the processing device 120, or through the network 150. As another example, the storage device 130 may be connected to the quantitative measurement device 110 directly as indicated by the bi-directional arrow in dotted lines linking the quantitative measurement device 110 and the storage device 130, or through the network 150. As still another example, the terminal device 140 may be connected to the processing device 120 directly as indicated by the bi-directional arrow in dotted lines linking the terminal device 140 and the processing device 120, or through the network 150.

The quantitative measurement device 110 may be configured to measure an abundance of one or more target metabolites for detecting whether the subject has CRC/ACRA. In some embodiments, the quantitative measurement device 110 may measure the abundance of the one or more target metabolites using a relative quantification approach or an absolute quantification approach. Merely by way of example, the quantitative measurement device 110 may include a mass spectrometer (MS; e.g., liquid chromatography-mass spectrometer, gas chromatography-mass spectrometer; matrix-assisted laser desorption/ionization time-of-flight mass spectrometer), an ultraviolet spectrometer, a High-Performance Liquid Chromatography (HPLC) apparatus, or the like.

The processing device 120 may process data and/or information obtained from the quantitative measurement device 110, the storage device 130, and/or the terminal device 140. In some embodiments, the processing device 120 may be used to process the quantified abundance of the one or more target metabolites for evaluating whether the subject has CRC/ACRA. For example, the processing device 120 may obtain a prediction model. The quantified abundance of the one or more target metabolites may be inputted into the prediction model to obtain a sample score for the subject. The processing device 120 may further evaluate whether the subject has CRC/ACRA by comparing the sample score to a cut-off value of the prediction model. In some embodiments, the processing device 120 may determine the quantified abundance of the one or more target metabolites based on data acquired by the quantitative measurement device 110.

In some embodiments, the processing device 120 may be a single server or a server group. The server group may be centralized or distributed. In some embodiments, the processing device 120 may be local or remote. For example, the processing device 120 may access information and/or data from the quantitative measurement device 110, the storage device 130, and/or the terminal device 140 via the network 150. As another example, the processing device 120 may be directly connected to the quantitative measurement device 110, the terminal device 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the terminal device 140. In some embodiments, the processing device 120 may be part of the quantitative measurement device 110.

The storage device 130 may store data, instructions, and/or any other information. In some embodiments, the storage device 130 may store data obtained from the quantitative measurement device 110, the processing device 120, and/or the terminal device 140. The data may include quantified abundance of the one or more target metabolites of the subject and/or the prediction model for processing the quantified abundance, etc. In some embodiments, the storage device 130 may store data and/or instructions that the processing device 120 may execute or use to perform exemplary methods described in the present disclosure. In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memories may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. some embodiments, the storage device 130 may be connected to the network 150 to communicate with one or more other components (e.g., the processing device 120, the terminal device 140) of the system 100. One or more components of the system 100 may access the data or instructions stored in the storage device 130 via the network 150. In some embodiments, the storage device 130 may be integrated into the quantitative measurement device 110 or the processing device 120.

The terminal device 140 may be connected to and/or communicate with the quantitative measurement device 110, the processing device 120, and/or the storage device 130. In some embodiments, the terminal device 140 may include a mobile device 141, a tablet computer 142, a laptop computer 143, or the like, or any combination thereof. For example, the mobile device 141 may include a mobile phone, a personal digital assistant (PDA), or the like, or any combination thereof. In some embodiments, the terminal device 140 may include an input device, an output device, etc. The input device may include alphanumeric and other keys that may be input via a keyboard, a touchscreen (e.g., with haptics or tactile feedback), a speech input, an eye-tracking input, a brain monitoring system, or any other comparable input mechanism. Other types of input devices may include a cursor control device, such as a mouse, a trackball, or cursor direction keys, etc. The output device may include a display, a printer, or the like, or any combination thereof. The terminal device 140 may be used to present information to a user and/or convey a user instruction to other components of the system 100. For example, the user (e.g., a doctor) may instruct the quantitative measurement device 110 to start quantifying the abundance of the one or more target metabolites via the terminal device 140. As another example, the user may view an evaluation result regarding whether the subject has CRC/ACRA via the terminal device 140.

The network 150 may include any suitable network that can facilitate the exchange of information and/or data for the system 100. In some embodiments, one or more components (e.g., the quantitative measurement device 110, the processing device 120, the storage device 130, the terminal device 140) of the system 100 may communicate information and/or data with one or more other components of the system 100 via the network 150.

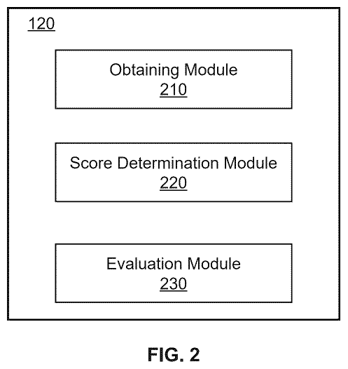

FIG. 2 is a block diagram illustrating an exemplary processing device according to some embodiments of the present disclosure. In some embodiments, the processing device 120 may include an obtaining module 210, a score determination module 220, and an evaluation module 230. In some embodiments, the modules may be hardware circuits of all or part of the processing device 120. The modules may also be implemented as an application or set of instructions read and executed by the processing device 120. Further, the modules may be any combination of the hardware circuits and the application/instructions. For example, the modules may be part of the processing device 120 when the processing device 120 is executing the application/set of instructions. In some embodiments, the processing device 120 may include a processor implemented on the terminal device 140.

The obtaining module 210 may obtain, from a quantitative measurement device, quantified abundance of one or more target metabolites in a panel of a plurality of metabolites in a sample from the subject.

The score determination module 220 may determine a sample score by processing the quantified abundance of each of the one or more target metabolites using a prediction model.

The evaluation module 230 may estimate whether the subject has CRC/ACRA by comparing the sample score to a cut-off score.

According to yet another aspect of the present disclosure, a method of detecting CRC/ACRA in a subject is provided. The method may include: (a) obtaining, from a quantitative measurement device, quantified abundance of one or more target metabolites in a panel of a plurality of metabolites in a sample from the subject, wherein the plurality of metabolites include the metabolites of Table 1; (b) determining a sample score by processing the quantified abundance of each of the one or more target metabolites using a prediction model; (c) evaluating whether the subject has CRC/ACRA by at least comparing the sample score to a cut-off score. In some embodiments, the method may be performed by the processing device 120 and/or one or more modules illustrated in FIG. 2.

In some embodiments, the subject is human. The sample may be a blood serum sample.

In some embodiments, the method of detecting CRC/ACRA in a subject may be used for discriminating subjects having CRC/at a high risk of having CRC from normal subjects. The normal subjects may have normal polyps and adenomas which do not exhibit a tendency to become malignant. Alternatively, the normal subjects may have no polyps or adenomas. The subjects having CRC may have early-stage CRC, or advanced CRC. The subjects at a high risk of having CRC may suffer from ACRA which tends to lead to CRC.

In some embodiments, the method may further include treating the subject. For example, the method may include steps (a)-(c) and further includes step (d): in response to determining that the subject has CRC/ACRA, applying a treatment to the subject. The treatment may include colectomy, ostomy, radiotherapy, pharmacotherapy, or a surgery for removing the ACRA or a tumor in the subject, or the like or any combination thereof. The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-4, and/or one or more metabolic combinations in Table 5.

The term “treatment of CRC/ACRA,” as used herein, refers to partially or totally inhibiting, delaying, or preventing the progression of colorectal cancer; inhibiting, delaying, or preventing the recurrence of cancer including cancer metastasis;

preventing the onset or development of cancer (chemoprevention) in the subject; and/or removing the ACRA. In some embodiments, the treatment may include palliative care. For instance, when the subject is diagnosed with late-stage CRC and other treatment approaches turn out to be ineffective, palliative care may be provided for the subject to mitigate the pain and stress of the subject.

In some embodiments, the method for detecting CRC/ACRA in a subject and treating the subject may further include: in response to an estimation that the subject has CRC/ACRA based on a comparison result of comparing the sample score to the cut-off score, verifying that the subject has CRC/ACRA with a diagnostic approach, such as colonoscopy, a biopsy test, a computerized tomography (CT) scan, a magnetic resonance imaging (MRI) scan, a positron emission tomography (PET) scan, or the like, or any combination thereof.

In some embodiments, the method for detecting CRC/ACRA in a subject may be especially suitable for subjects to whom coloscopy or other invasive approaches are not appropriate. For instance, if a subject has a heart failure, a respiratory failure, an acute gastrointestinal perforation, or a mental illness, etc., the coloscopy examination may not be suitable for the subject. Thus, the method for detecting CRC/ACRA provided by the present disclosure may be adopted for subjects like this.

In some embodiments, the method for detecting CRC/ACRA in a subject provided by the present disclosure may be used as a pre-examination for the subject before a coloscopy. Since the coloscopy is an invasive approach and causes much discomfort and pain to the subject, it is desired to eliminate unnecessary coloscopy examinations. For example, according to an evaluation result of the method for detecting CRC/ACRA in the subject using the one or more target metabolites, if the subject is evaluated as not having CRC/ACRA, the coloscopy examination may be unnecessary for the subject; if the subject is evaluated as having CRC/ACRA, a coloscopy examination and/or other approaches for further diagnosis of CRC or ACRA may be conducted for the subject.

According to yet another aspect of the present disclosure, a method of detecting stage I/II CRC in a subject is provided. The method may include: (a) obtaining, from a quantitative measurement device, quantified abundance of one or more target metabolites in a panel of a plurality of metabolites in a sample from the subject; (b) determining a sample score by processing the quantified abundance of each of the one or more target metabolites using a prediction model; and (c) estimating whether the subject has stage I/II CRC by comparing the sample score to a cut-off score. The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-4, and/or one or more metabolic combinations in Table 5.

In some embodiments, step b) may further include normalizing the abundance of each of the metabolites quantified in step (a), and determining the sample score by processing the normalized abundance with a prediction model. The prediction model may be established using a plurality of training datasets. For example, each of the plurality of training datasets may include the abundance of a metabolite of a reference subject and a label indicating whether the reference subject has a stage I/II CRC or is normal. The plurality of reference subjects may include a plurality of normal subjects who do not have CRC and a plurality of subjects having a stage I/II CRC.

According to still another aspect of the present disclosure, a method of detecting advanced CRC (ACRC) in a subject is provided. As used herein, the term “ACRC” refers to stage III/IV CRC. The method may include: (a) obtaining, from a quantitative measurement device, quantified abundance of one or more target metabolites in a panel of a plurality of metabolites in a sample from the subject; (b) determining a sample score by processing the quantified abundance of each of the one or more target metabolites using a prediction model; and (c) estimating whether the subject has advanced CRC by comparing the sample score to a cut-off score. The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-4, and/or one or more metabolic combinations in Table 5.

In some embodiments, step b) may further include normalizing the abundance of each of the metabolites quantified in step (a), and determining the sample score by processing the normalized abundance with a prediction model. The prediction model may be established using a plurality of training datasets. For example, each of the plurality of training datasets may include the abundance of a metabolite of a reference subject and a label indicating whether the reference subject has a stage III/IV CRC or is normal. The plurality of reference subjects may include a plurality of normal subjects who do not have CRC and a plurality of subjects having stage III/IV CRC.

According to yet another aspect of the present disclosure, a method of detecting ACRA in a subject is provided. As used herein, the term “ACRA” refers to stage III/IV CRC. The method may include: (a) obtaining, from a quantitative measurement device, quantified abundance of one or more target metabolites in a panel of a plurality of metabolites in a sample from the subject; (b) determining a sample score by processing the quantified abundance of each of the one or more target metabolites using a prediction model; and (c) estimating whether the subject has ACRA by comparing the sample score to a cut-off score. The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-4, and/or one or more metabolic combinations in Table 5.

In some embodiments, step b) may further include normalizing the abundance of each of the metabolites quantified in step (a), and determining the sample score by processing the normalized abundance with a prediction model. The prediction model may be established using a plurality of training datasets. For example, each of the plurality of training datasets may include the abundance of a metabolite of a reference subject and a label indicating whether the reference subject has ACRA or is normal. The plurality of reference subjects may include a plurality of normal subjects who do not have ACRA and a plurality of subjects having ACRA.

According to yet another aspect of the present disclosure, a use of one or more target diagnostic biomarkers for generating a trained machine-learning model for estimating whether a subject has CRC/ACRA is provided. The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-4, and/or one or more metabolic combinations in Table 5.

According to still another aspect of the present disclosure, a use of the one or more target metabolites for preparing a kit for detecting CRC/ACRA in as subject is provided. The description of the one or more target metabolites may be found earlier in the present disclosure.

According to yet another aspect of the present disclosure, a kit for detecting CRC/ACRA in a subject is provided. In some embodiments, the kit may include the one or more target metabolites. The description of the one or more target metabolites may be found earlier in the present disclosure. For example, the one or more target metabolites may be used as standard substances. The standard substances may be used for accurate determination of the abundance of the one or more target metabolites in the subject. Specifically, the standard substances may be used for generating one or more standard curves for quantifying the abundance of the one or more target metabolites in the subject. Additionally, the kit may also include other components which are not limited by the present disclosure, such as one or more quality-control agents, one or more pre-treatment agents for pre-treating the sample of the subject (e.g., a blood serum sample), or the like, or any combination thereof.

The metabolite annotations of Meta IDs used in the present disclosure and some features related to these metabolites are listed in Table 6.

TABLE 6

| Meta | | | Delta(pp |
|---|---|---|---|
| ID | MASS(+/−) | Compound | m) |
| BN001 | 319.228(−) | 5_HETE | 0 |
| BN002 | 319.228(−) | 15_HETE | 0 |
| BN003 | 319.228(−) | 8_HETE | 0 |
| BN004 | 319.228(−) | 9_HETE | 0 |
| BN005 | 319.228(−) | 11_HETE | 0 |
| BN006 | 319.228(−) | 12_HETE | 0 |
| BN010 | 313.239(−) | 9(10)_DiHOME | 0 |
| BN011 | 313.239(−) | 12(13)_DiHOME | 0 |
| BN012 | 343.228(−) | 14(S)-HDHA | 0 |
| BN015 | 350.210(−) | Sphingosine-1-phosphate (d16:1) | 0 |
| BN016 | 313.239(−) | Octadecane dioic acid | 0 |
| BN017 | 367.300(−) | Epitestosterone Sulfate | 0 |
| BN018 | 311.100(−) | (9E,11E)-13-Hydroperoxy-9,11-octadecadienoic | 0 |
| | | acid | |
| BN019 | 329.234(−) | 9(S), 10(S), 13(S)-Trihydroxy-10(E)-Octadecenoic | 0 |
| | | Acid | |
| BN020 | 389.270(−) | 3α-Hydroxy-6-OXO-5α-Cholan-24-OIC Acid | 0 |
| BN021 | 405.265(−) | 3-Dehydrocholic Acid | 0 |
| BN022 | 405.265(−) | 5α-Cholanic Acid-3α,7β-Diol-6-One | 0 |
| BN023 | 301.218(−) | Eicosapentaenoic Acid | 0 |
| BN025 | 361.202(−) | Hydrocortisone | 0 |
| BN027 | 480.310(−) | 1-Stearoyl-2-Hydroxy-sn-Glycero-3- | 0 |
| | | Phosphoethanolamine | |
| BN028 | 159.067(−) | Pimelic acid | 0 |
| BN029 | 187.098(−) | Azelaic acid | 0 |
| BN030 | 303.233(−) | Arachidonic acid | 0 |
| BN031 | 173.119(−) | 3-Hydroxynonanoic acid | 0 |
| BN032 | 471.235(−) | Chenodeoxycholic Acid-3-Sulfate Sodium Salt | 0 |
| BN034 | 452.279(−) | 1-Palmitoyl-2-hydroxy-sn-glycero-3-PE | 0 |
| BN035 | 329.234(−) | 9(S),10(S),13(S)_Trihydroxy_11(E)_Octadecenoic | 0 |
| | | Acid | |
| BP001 | 190.086(+) | 3-Indolepropionic acid | 0 |
| BP002 | 177.102(+) | S-(−)-Cotinine | 0 |
| BP003 | 379.284(+) | 2-Arachidonoyl Glycerol | 0 |
| BP006 | 372.311(+) | Myristoyl-L-carnitine | 0 |
| BP007 | 286.201(+) | trans-2-octenoyl-I-carnitine | 0 |
| BP008 | 246.170(+) | 2-Methylbutyryl-L-carnitine | 0 |
| BP009 | 355.284(+) | 1-Linoleoyl-rac-glycerol | 0 |
| BP010 | 398.326(+) | trans-2-Hexadecenoyl-L-carnitine | 0 |
| BP011 | 316.248(+) | Decanoyl-L-carnitine | 0 |
| BP012 | 261.193(+) | (±)-Hexanoyl carnitine chloride | 0 |
| BP013 | 303.232(+) | 17-Alpha-Methyltestosterone | 0 |
| C001 | 229.144(−) | C12H22O4 | 2 |
| C002 | 245.050(−) | C13H10O5 | 17 |
| C004 | 267.073(−) | C10H12N4O5 | 2 |
| C005 | 271.228(−) | C16H32O3 | 2 |
| C006 | 295.229(−) | C18H32O3 | 2 |
| C008 | 327.256(−) | C19H36O4 | 5 |
| C009 | 355.158(−) | C22H20N4O | 4 |
| C010 | 374.132(−) | C22H18FN3O2 | 3 |
| C011 | 381.174(−) | C16H30O10 | 7 |
| C012 | 398.132(−) | C19H21N5O3S | 7 |
| C013 | 399.221(−) | C21H36O5S | 0 |
| C014 | 400.148(−) | C21H24FN3O2S | 5 |
| C015 | 407.280(−) | C24H40O5 | 0 |
| C016 | 427.163(−) | C24H23F3N2O2 | 2 |
| C017 | 439.379(−) | C27H52O4 | 0 |
| C019 | 447.312(−) | C27H44O5 | 1 |
| C020 | 465.249(−) | C25H38O8 | 1 |
| C021 | 468.308(−) | C29H43NO2S | 30 |
| C022 | 469.227(−) | C20H38O12 | 4 |
| C023 | 469.227(−) | C21H34N4O8 | 7 |
| C024 | 471.242(−) | C24H40O7S | 0 |
| C025 | 480.308(−) | C25H43N3O6 | 0 |
| C026 | 480.310(−) | C25H43N3O6 | 4 |
| C027 | 481.354(−) | C27H48O4 | 1 |
| C028 | 507.273(−) | C24H45O9P | 0 |
| C029 | 510.253(−) | C29H33N7O2 | 18 |
| C030 | 528.310(−) | C27H48NO7P | 1 |
| C031 | 540.331(−) | C34H43N3O3 | 14 |
| C032 | 580.235(−) | C33H35N5O5 | 37 |
| C033 | 581.241(−) | C33H34N4O6 | 1 |
| C034 | 584.357(−) | C37H47NO5 | 32 |
| C035 | 590.346(−) | C33H45N5O5 | 19 |
| C036 | 642.396(−) | C31H57N5O9 | 19 |
| C037 | 204.982(−) | C7H10OS3 | 1 |
| C038 | 279.233(−) | C18H32O2 | 0 |
| C040 | 329.248(−) | C22H34O2 | 0 |
| C041 | 499.288(−) | C26H44O9 | 7 |
| C042 | 511.302(−) | C31H44O6 | 9 |
| C043 | 511.302(−) | C24H49O9P | 4 |
| C102 | 181.072(+) | C7H8N4O2 | 1 |
| C105 | 246.170(+) | C12H23NO4 | 0 |
| C108 | 271.190(+) | C15H26O4 | 1 |
| C110 | 286.201(+) | C15H27NO4 | 1 |
| C112 | 315.134(+) | C17H18N2O4 | 0 |
| C113 | 315.134(+) | C15H17F3N2O2 | 9 |
| C114 | 317.195(+) | C19H37NO6 | 3 |
| C115 | 317.195(+) | C16H28O6 | 3 |
| C116 | 330.263(+) | C18H35NO4 | 2 |
| C118 | 331.262(+) | C22H34O2 | 1 |
| C119 | 337.273(+) | C21H36O3 | 2 |
| C120 | 355.283(+) | C21H38O4 | 4 |
| C121 | 358.295(+) | C20H39NO4 | 1 |
| C122 | 372.300(+) | C24H37NO2 | 28 |
| C124 | 398.325(+) | C23H43NO4 | 4 |
| C127 | 426.357(+) | C25H47NO4 | 2 |
| C128 | 428.363(+) | C25H48O5 | 23 |
| C129 | 442.352(+) | C25H47NO5 | 2 |
| C130 | 452.277(+) | C21H42NO7P | 0 |
| C131 | 468.308(+) | C22H46NO7P | 1 |
| C132 | 480.134(+) | C23H21N5O5S | 1 |
| C134 | 518.368(+) | C14H22N2O2 | 4 |
| C135 | 195.087(+) | C8H10N4O2 | 2 |
| C136 | 287.204(+) | C19H26O2 | 14 |
| C137 | 302.215(+) | C19H27NO2 | 11 |
| C138 | 302.233(+) | C16H31NO4 | 2 |
| C139 | 341.306(+) | C21H40O3 | 2 |
| C140 | 341.306(+) | C23H36N2 | 31 |
| C141 | 355.227(+) | C23H30O3 | 1 |
| C142 | 357.243(+) | C20H24O2 | 0 |
| C143 | 357.243(+) | C21H31F3O | 9 |
| C144 | 357.280(+) | C24H36O2 | 3 |
| C145 | 464.314(+) | C23H46NO6P | 2 |
| C146 | 482.324(+) | C23H48NO7P | 0 |
| C147 | 506.323(+) | C25H47NO9 | 18 |
| C148 | 508.340(+) | C25H50NO7P | 1 |
| C149 | 508.340(+) | C28H45NO7 | 26 |
| C150 | 530.324(+) | C27H48NO7P | 1 |
| DS01 | 407.281(−) | CA (Cholic Acid) | 0 |
| DS02 | 391.286(−) | CDCA (Chenodeoxycholic Acid) | 0 |
| DS03 | 391.286(−) | DCA (Deoxycholic Acid) | 0 |
| DS04 | 464.302(−) | GCA (Glycocholic Acid Hydrate) | 0 |
| DS05 | 448.307(−) | GCDCA (Glycochenodeoxycholic Acid) | 0 |
| DS06 | 448.307(−) | GDCA (Glycodeoxycholic Acid) | 0 |
| DS07 | 432.312(−) | GLCA (Glycolithocholic Acid) | 0 |
| DS08 | 448.307(−) | GUDCA (Glycoursodeoxycholic Acid) | 0 |
| DS09 | 514.693(−) | TCA (Taurocholic Acid) | 0 |
| DS10 | 391.286(−) | UDCA (Ursodeoxycholic Acid) | 0 |
| DS11 | 391.300(−) | 5β-CAA-3β,12α-2K | 0 |
| X004 | 239.092(−) | C12H16O5 | 2 |
| X006 | 263.104(−) | C13H16N2O4 | 1 |
| X010 | 299.259(−) | C17H34O | 1 |
| X011 | 311.223(−) | C18H32O4 | 1 |
| X012 | 313.119(−) | C17H18N2O4 | 1 |
| X013 | 313.238(−) | C18H34O4 | 1 |
| X016 | 319.228(−) | C20H32O3 | 0 |
| X019 | 343.228(−) | C22H32O3 | 0 |
| X023 | 367.158(−) | C19H28O5S | 1 |
| X024 | 369.174(−) | C19H30O5S | 0 |
| X032 | 403.158(−) | C25H24O5 | 7 |
| X036 | 425.201(−) | C23H27FN4O3 | 4 |
| X042 | 452.278(−) | C21H44NO7P | 1 |
| X043 | 453.232(−) | C20H38O11 | 5 |
| X051 | 504.310(−) | C32H43NO4 | 4 |
| X055 | 526.315(−) | C23H48NO7P | 0 |
| X066 | 592.362(−) | C29H56NO9P | 0 |
| X068 | 624.339(−) | C32H51NO11 | 0 |
| X070 | 646.427(−) | C34H66NO8P | 28 |
| X082 | 353.212(−) | C19H26N6O | 7 |
| X133 | 143.07(+) | C7H10O3 | 2 |
| X139 | 217.143(+) | C11H20O4 | 2 |
| X141 | 231.174(+) | C11H22N2O3 | 16 |
| X144 | 257.174(+) | C17H33NO4 | 3 |
| X152 | 300.216(+) | C18H25N3O | 30 |
| X154 | 302.196(+) | C16H23N5O | 5 |
| X155 | 303.231(+) | C20H30O2 | 3 |
| X160 | 316.247(+) | C17H33NO4 | 4 |
| X166 | 352.224(+) | C16H34NO5P | 2 |
| X178 | 432.311(+) | C26H43NO5 | 1 |
| X183 | 490.300(+) | C13H17N3O | 5 |
| X188 | 542.324(+) | C28H48NO7P | 0 |
| X196 | 347.196(+) | C21H24N4O2 | 25 |
| X278 | 289.106(−) | C11H18N2O7 | 6 |
| X280 | 447.312(−) | C29H46O | 19 |
| X281 | 447.312(−) | C50H89O11P | 27 |
| X285 | 512.336(−) | C28H47NO6 | 5 |
| X286 | 512.336(−) | C29H49NO5 | 1 |
| X289 | 536.299(−) | C31H41NO4 | 5 |
| X292 | 187.007(−) | C7H8O4S | 0 |
| X293 | 204.067(−) | C11H11NO3 | 2 |
| X314 | 637.157(−) | C32H31O14 | 0 |
| X401 | 222.114(−) | C12H17NO3 | 2 |
| X402 | 229.144(−) | C10H18O2 | 2 |
| X403 | 239.092(−) | C13H12N4O | 8 |
| X405 | 279.233(−) | C17H30 | 0 |
| X407 | 314.103(−) | C17H17NO5 | 1 |
| X408 | 317.212(−) | C20H30O3 | 1 |
| X409 | 335.259(−) | C20H34O | 0 |
| X411 | 345.243(−) | C22H34O3 | 2 |
| X412 | 353.164(−) | C19H22N4O3 | 6 |
| X415 | 369.227(−) | C20H34O6 | 3 |
| X416 | 374.132(−) | C20H25NO2S2 | 18 |
| X419 | 229.108(−) | C12H22S2 | 4 |
| X421 | 362.030(−) | C10H14N5O6PS | 8 |
| X422 | 168.103(−) | C9H15NO2 | 0 |
| X423 | 173.118(−) | C9H18O3 | 2 |
| X424 | 297.983(−) | C9H11NO6S | 12 |
| X425 | 362.030(−) | C16H12CI2FN5 | 22 |
| X426 | 201.113(−) | C10H18O4 | 1 |
| X427 | 389.181(−) | C25H26O4 | 13 |
| X428 | 260.158(−) | C13H19N5O | 24 |
| X429 | 445.114(−) | C22H22O10 | 0 |
| X430 | 431.228(−) | C21H36O9 | 2 |
| X431 | 445.244(−) | C23H34N4O5 | 4 |
| X432 | 183.103(−) | C10H16O3 | 2 |
| X433 | 201.113(−) | C11H14N4 | 8 |
| X434 | 287.186(−) | C11H24N6O3 | 8 |
| X435 | 201.150(−) | C11H22O3 | 2 |
| X436 | 401.218(−) | C20H34O8 | 0 |
| X437 | 301.202(−) | C16H30O5 | 0 |
| X438 | 415.234(−) | C22H32N4O4 | 3 |
| X439 | 415.234(−) | C23H28N8 | 6 |
| X440 | 487.291(−) | C31H40N2O3 | 12 |
| X441 | 429.249(−) | C24H34N2O5 | 22 |
| X442 | 575.175(−) | C28H32O13 | 4 |
| X443 | 429.249(−) | C28H31FN2O | 33 |
| X444 | 443.265(−) | C23H40O8 | 0 |
| X445 | 659.366(−) | C33H57O11P | 14 |
| X446 | 457.280(−) | C28H42O5 | 35 |
| X447 | 602.203(−) | C32H28F3N5O4 | 2 |
| X448 | 329.233(−) | C18H34O5 | 1 |
| X449 | 687.398(−) | C39H61O8P | 7 |
| X450 | 629.355(−) | C32H54O12 | 1 |
| X451 | 715.429(−) | C41H65O8P | 8 |
| X452 | 713.414(−) | C37H62O13 | 3 |
| X453 | 716.431 (−) | C15H17N3 | 16 |
| X454 | 671.402(−) | C35H61O10P | 13 |
| X455 | 685.419(−) | C37H67O9P | 38 |
| X456 | 699.434(−) | C38H69O9P | 38 |
| X457 | 463.343(−) | C28H48O5 | 0 |
| X458 | 299.259(−) | C18H36O3 | 1 |
| X459 | 930.431(−) | C43H73N3O16P2 | 3 |
| X460 | 229.108(−) | C18H14 | 25 |
| X461 | 346.035(−) | C14H7F6N3O | 20 |
| X462 | 390.025(−) | C18H15N3O3S | 18 |
| X463 | 183.103(−) | C9H14O | 2 |
| X464 | 403.197(−) | C19H32O9 | 1 |
| X465 | 673.382(−) | C37H54O11 | 34 |
| X466 | 687.396(−) | C42H56O8 | 8 |
| X467 | 701.413(−) | C36H63O11P | 14 |
| X468 | 715.429(−) | C37H65O11P | 14 |
| X469 | 743.459(−) | C43H69O8P | 9 |
| X470 | 700.436(−) | C37H68NO9P | 28 |
| X471 | 713.450(−) | C39H71O9P | 37 |
| X472 | 758.481(−) | C43H70NO8P | 6 |
| X473 | 465.358(−) | C28H50O5 | 1 |
| X474 | 757.477(−) | C41H75O10P | 34 |
| X475 | 727.464(−) | C39H69O10P | 12 |
| X476 | 771.493(−) | C45H73O8P | 5 |
| X477 | 788.519(−) | C45H76NO8P | 6 |
| X478 | 785.508(−) | C41H75N2O10P | 1 |
| X479 | 799.524(−) | C42H77N2O10P | 0 |
| X480 | 955.601(−) | C51H89O14P | 10 |
| X508 | 212.020(+) | C7H2F5NO | 33 |
| X509 | 225.148(+) | C13H20O3 | 2 |
| X511 | 263.200(+) | C17H26O2 | 2 |
| X512 | 263.236(+) | C18H30O | 4 |
| X513 | 305.247(+) | C20H32O2 | 2 |
| X515 | 335.151(+) | C19H18N4O2 | 2 |
| X516 | 339.162(+) | C21H22O4 | 9 |
| X517 | 350.153(+) | C18H24NO4P | 4 |
| X518 | 363.162(+) | C21H19FN4O | 1 |
| X519 | 364.084(+) | C13H14O11 | 9 |
| X523 | 505.336(+) | C26H48O9 | 2 |
| X525 | 563.427(+) | C34H58O6 | 6 |
| X531 | 1024.676(+) | C52H97NO18 | 2 |
| X532 | 477.307(+) | C25H40N4O5 | 0 |
| X533 | 121.101(+) | C9H12 | 1 |
| X534 | 243.159(+) | C13H22O4 | 0 |
| X535 | 224.165(+) | C13H21NO2 | 2 |
| X536 | 193.122(+) | C12H16O2 | 2 |
| X537 | 226.144(+) | C12H19NO3 | 1 |
| X538 | 500.343(+) | C27H41N5O4 | 40 |
| X539 | 544.369(+) | C30H51NO6 | 15 |
| X540 | 484.348(+) | C25H52NO4P | 10 |
| X541 | 255.159(+) | C14H22O4 | 0 |
| X542 | 207.138(+) | C13H18O2 | 0 |
| X543 | 447.259(+) | C24H30N8O | 6 |
| X544 | 500.170(+) | C26H29NO9 | 43 |
| X545 | 253.180(+) | C15H24O3 | 1 |
| X546 | 271.190(+) | C14H22O3 | 1 |
| X547 | 289.201(+) | C15H28O5 | 0 |
| X548 | 306.227(+) | C22H27N | 18 |
| X549 | 311.183(+) | C12H26N2O7 | 6 |
| X550 | 630.306(+) | C33H45N5O5 | 1 |
| X551 | 341.232(+) | C19H32O5 | 1 |
| X552 | 239.164(+) | C14H22O3 | 1 |
| X553 | 425.253(+) | C23H36O7 | 1 |
| X554 | 215.128(+) | C11H18O4 | 1 |
| X555 | 385.222(+) | C20H32O7 | 0 |
| X556 | 420.259(+) | C27H33NO3 | 14 |
| X557 | 456.144(+) | C20H29N3O5S2 | 40 |
| X558 | 395.300(+) | C27H38O2 | 14 |
| X559 | 457.279(+) | C24H40O6S | 38 |
| X560 | 589.435(+) | C36H60O6 | 19 |
| X561 | 544.406(+) | C37H53O4 | 25 |
| X562 | 476.294(+) | C27H41NO6 | 14 |
| X563 | 271.190(+) | C18H35NO4 | 1 |
| X564 | 457.280(+) | C28H32N4O2 | 44 |
| X565 | 629.447(+) | C35H64O9 | 24 |
| X566 | 602.448(+) | C32H54O5 | 7 |
| X567 | 514.395(+) | C29H54O7 | 28 |
| X568 | 471.296(+) | C26H38N4O4 | 1 |
| X569 | 602.447(+) | C34H61NO6 | 13 |
| X570 | 558.421(+) | C18H26O | 17 |
| X571 | 514.395(+) | C29H52O6 | 30 |
| X572 | 470.369(+) | C27H51NO3S | 6 |
| X573 | 405.301(+) | C25H40O4 | 3 |
| X574 | 748.542(+) | C40H78NO9P | 9 |
| X575 | 792.568(+) | C42H82NO10P | 9 |
| X576 | 660.489(+) | C43H65NO4 | 15 |
| X577 | 485.311(+) | C26H44O8 | 0 |
| X578 | 704.516(+) | C38H74NO8P | 9 |
| X579 | 599.437(+) | C37H58O6 | 11 |
| X580 | 617.467(+) | C38H64O6 | 17 |
| X581 | 660.489(+) | C35H66NO8P | 44 |
| X582 | 507.336(+) | C28H46N2O6 | 14 |
| X583 | 572.437(+) | C36H61NO4 | 53 |
| X584 | 511.384(+) | C29H51O5P | 57 |
| X585 | 528.411(+) | C16H26O2 | 2 |
| X586 | 616.463(+) | C38H65NO5 | 50 |
| X587 | 718.531(+) | C39H76NO8P | 10 |
| X588 | 528.411(+) | C29H48O3 | 8 |
| X589 | 701.505(+) | C39H73O8P | 9 |
| X590 | 512.207(+) | C20H33NO14 | 19 |
| X591 | 674.505(+) | C37H72NO7P | 10 |
| X592 | 630.479(+) | C38H63NO6 | 10 |
| X593 | 586.452(+) | C37H63NO4 | 53 |
| X594 | 820.601(+) | C44H86NO10P | 6 |
| X595 | 776.573(+) | C42H82NO9P | 9 |
| X596 | 550.390(+) | C28H56NO7P | 6 |
| X597 | 732.547(+) | C40H78NO8P | 9 |
| X598 | 514.377(+) | C31H46O2 | 22 |
| X599 | 688.521(+) | C38H74NO7P | 10 |
| X600 | 671.494(+) | C37H67O8P | 44 |
| X601 | 644.494(+) | C36H69NO8 | 24 |
| X602 | 543.398(+) | C34H54O5 | 12 |
| X603 | 521.384(+) | C31H52O6 | 1 |
| X604 | 720.453(+) | C36H65NO13 | 0 |
| X605 | 878.640(+) | C50H88NO9P | 15 |
| X606 | 645.385(+) | C33H57O10P | 14 |
| X607 | 662.411(+) | C31H51N9O7 | 19 |
| X608 | 600.468(+) | C38H65NO4 | 51 |
| X609 | 746.562(+) | C41H80NO8P | 10 |
| X610 | 717.442(+) | C38H69O10P | 37 |
| X611 | 734.469(+) | C41H68NO8P | 9 |
| X612 | 659.400(+) | C35H63O9P | 43 |
| X613 | 676.426(+) | C34H62NO10P | 11 |
| X614 | 673.416(+) | C35H61O10P | 13 |
| X615 | 573.363(+) | C26H48N6O8 | 4 |
| X616 | 404.301(+) | C17H37N7O4 | 7 |
| X617 | 687.431(+) | C36H63O10P | 11 |
| X618 | 513.342(+) | C32H48O5 | 30 |
| X619 | 701.447(+) | C37H65O10P | 12 |
| X620 | 718.474(+) | C38H72NO9P | 39 |
| X621 | 401.290(+) | C26H40O3 | 37 |
| X622 | 745.473(+) | C39H69O11P | 11 |
| X623 | 759.489(+) | C40H71O11P | 11 |
| X624 | 485.311(+) | C27H40N4O4 | 3 |
| X625 | 776.515(+) | C40H74NO11P | 10 |
| X626 | 779.524(+) | C44H75O9P | 2 |
| X627 | 513.342(+) | C14H24O4 | 0 |
| X628 | 571.384(+) | C31H54O9 | 0 |
| X629 | 715.463(+) | C38H67O10P | 12 |
| X630 | 741.478(+) | C40H69O10P | 11 |
| X631 | 812.400(+) | C41H70NO8P | 4 |
| X632 | 227.200(+) | C14H26O2 | 2 |
| X633 | 397.295(+) | C23H4005 | 0 |
| X634 | 415.305(+) | C25H38N2O3 | 23 |
| X635 | 432.332(+) | C27H45NO3 | 35 |
| X636 | 773.505(+) | C41H73O11P | 11 |
| X637 | 787.520(+) | C42H75O11P | 10 |
| X638 | 425.253(+) | C18H32N8O4 | 21 |
| X639 | 690.463(+) | C35H58O8 | 8 |
| X640 | 741.478(+) | C41H73O9P | 38 |
| X641 | 157.122(+) | C9H16O2 | 2 |

The methods and metabolite biomarkers provided by the present disclosure are further described according to the following examples, which should not be construed as limiting the scope of the present disclosure. More description regarding the performance of some exemplary prediction models based on the one or more target metabolites may also be found in the following examples. As shown in these Examples, the AUC, specificity, and sensitivity of the prediction models are relatively high, indicating that the prediction model utilizing the abundance of these metabolites may effectively distinguish subjects with CRC/ACRA from normal subjects.

### Examples

### Material and Method

### 1. Cohort Composition

From 2019 to 2022, 701 consecutive serum samples from 4 independent centers were collected before coloscopy, and were subjected to targeted metabolomics detection. An optical colonoscopy examination was carried out for each individual in this cohort, and histopathological diagnosis of all significant lesions discovered during the colonoscopy were also been assessed. Subjects with no findings were categorized as negative by colonoscopy. Histopathological results from biopsied tissue or excised lesions were categorized based on the most clinically significant lesion present (i.e., the index lesion) by a central pathologist according to the pre-specified standards outlined in the Table below. Staging of CRC individuals was based on the tumor size, node, and metastasis staging system maintained by the American Joint Committee on Cancer and the International Union for Cancer Control. The number of individuals allocated into each category according to histopathological analysis is also listed in the table 1 below. Among them, normal individuals and individuals having colorectal polyps and low-risk adenoma were classified into the negative group, while individuals having advanced adenoma, or early/advanced CRC were classified into the positive group.

TABLE 7
Grouping standards and cohort composition of this cohort.

| | | | | Age | | |
|---|---|---|---|---|---|---|
| | | Definition of | Num- | (mean ± | | |
| Group | Category | categories | ber | SD) | Gender | Total |
| nega- | normal | no findings on | 185 | 55.6 ± | male: 89 | 234 |
| tive | | colonoscopy | | 7.8 | female: 96 | |
| | colorectal | inflammatory or | 21 | 57.6 ± | male: 15 | |
| | polyps | hyperplastic | | 10.6 | female: 6 | |
| | | polyps | | | | |
| | low-risk | 1 or 2 | 28 | 62.1 ± | male: 20 | |
| | adenoma | adenoma(s), | | 8.4 | female: 8 | |
| | | ≤10 mm in size, | | | | |
| | | non-advanced | | | | |
| posi- | advanced | Advance | 83 | 61.6 ± | mael: 57 | 467 |
| tive | adenoma | adenoma, | | 8.5 | female: 26 | |
| | | including | | | | |
| | | the following | | | | |
| | | subcategories: | | | | |
| | | Adenoma with | | | | |
| | | carcinoma in | | | | |
| | | situ/high-grade | | | | |
| | | dysplasia, | | | | |
| | | any size | | | | |
| | | Adenoma, | | | | |
| | | villous | | | | |
| | | growth pattern | | | | |
| | | (>25%), | | | | |
| | | any size | | | | |
| | | Adenoma | | | | |
| | | >1.0 cm in | | | | |
| | | size, or | | | | |
| | | 2.4-Serrated | | | | |
| | | lesion, >1.0 cm | | | | |
| | | in size | | | | |
| | early- | CRC stage 0-1 | 86 | 60.5 ± | male: 56 | |
| | stage | | | 8.4 | female: 30 | |
| | CRC | | | | | |
| | advanced | CRC stage | 298 | 63.3 ± | male: 174 | |
| | stage | II-IV | | 9.2 | female: 124 | |
| | CRC | | | | | |
| Total | | | | | | 701 |

### 2. Reagents and Equipment

### Equipment

Vortex mixer (Kylin-Bell Vortex X5)
20 μL, 100 μL, 200 μL, 1000μ L Pipettes and tips (Gilson)
High-speed microcentrifuge (Centrifuge 5415R)
Electronic balance (Mettler-Toledo AB104)
Centrifugal vacuum evaporator (TOMY CC-105)
Exion-20adxr Ultra Performance Liquid Chromatography system (Shimadzu) coupled with a Triple Quad™ 4500MD LC-MS/MS system (AB Sciex)
ACQUITY UPLC BEH C18 Column (Shim-pack Velox C18 2.7 μm 2.1×100 mm)
R statistical scripting language (version 4.2.1)
AB Sciex Analyst software system (version 1.6.3)

### Reagents and Supplies

LC-MS-grade methanol (Thermo Fisher Scientific)
LC-MS-grade acetonitrile (Thermo Fisher Scientific)
LC-MS-grade formic acid (Thermo Fisher Scientific)
Ammonium acetate, LC-MS grade (Thermo Fisher Scientific)
13C cholic acid (Sigma-Aldrich)
Ultrapure water, HPLC grade (Watsons)
Centrifuge tubes (1.5 ml; Axygen, cat. no. MCT-150-C)
10 μL, 200 μL, 1000μ L Pipette tips (Axygen)

### Solutions

13C labeled cholic acid stock solution (internal standard): weigh 10.8 mg cholic acid and dissolve into 1080 μL methanol, violently vortex until total dissolution. The final concentration of the stock solution is 10 mg/ml.

Precipitation solution: add 120 ul 13C cholic acid stock solution into 300 ml methanol and mix.

### 3. Metabolites Extraction

For metabolite extraction in targeted metabolomics detection, 10 μL internal standard solution (5 μg/mL 13C-Cholic Acid) was added to 80 μL serum with 150 μL acetonitrile: isopropanol (4:1 by volume, Thermo Fisher), 50 μL ammonium formate (0.5 g/mL), vortexing and followed by centrifugation at 17,949 g for 5 min. Then, 60 μL supernatant was diluted with 150 μL HPLC-grade water before use.

### 4. Targeted Metabolomics Detection

### I. Detection Method

The pseudo-targeted method independent of pure standards is developed, similar to what has been described by Fujian Zheng et. al (Nature Protocols, 2020), determining the relative level of all metabolites in the identified panel by using the same reference pool sample for normalizing abundances for each individual. Targeted metabolomics detection was carried out on AB SCIEX Triple Quad™ 4500 system and run in separate ion modes (positive and negative). The mobile phase and the column used for reversed-phase liquid chromatography were used as listed in the table below. The injection volume was 15 μL for each mode. Metabolites were eluted from the column at a flow rate of 0.3 ml/min with a gradually increasing concentration of mobile phase B, 12% of mobile phase B initially, to 60% of the mobile phase B after 2.5 min. A linear 60%-85% and 85%-100% phase B gradient was set at 6 min and 8.5 min. The quality control samples of the targeted analysis were pooled as follows: I-pool: C pool (1:1). Delustering potentials and collision energies were optimized from the quality control samples of the control group. Metabolite peaks were integrated using the Sciex Analyst 1.6.3 software.

### II. Chromatography Parameters

TABLE 8
Details of chromatography parameters.

Positive mode

| Column | Shimadzu shim-pack Velox C18(50*2.1 mm, 2.7 μm) | | | |
|---|---|---|---|---|
| Temperature | 45° C. | | | |
| Autosampler temperature | 15° C. | | | |
| Mobile phase | A: 0.1% formic acid-water | | | |
| | B: 0.1% formic acid-acetonitrile | | | |
| | | | mobile | mobile |
| | | flow | phase | phase |
| | time(min) | rate(ml/min) | A (%) | B (%) |
| Mobile phase | 0.50 | 0.4 | 65~75 | 25~30 |
| gradient | 2.50 | 0.4 | 45~55 | 45~55 |
| | 3.70 | 0.4 | 20~25 | 75~85 |
| | 3.71 | 0.4 | 1~5 | 95~100 |
| | 4.99 | 0.4 | 1~5 | 95~100 |
| | 5.00 | 0.4 | 78~82 | 18~22 |
| | 6.00 | 0.4 | 78~82 | 18~22 |

| | |
|---|---|
| Autosampler wash | 80% formic acid |
| Sample size | 15 μL |
| Pre-balance before | inject the wash solution once |
| sample injection | |

Negative mode

| Column | Shimadzu shim-pack Velox C18(50*2.1 mm, 2.7 μm) | | | |
|---|---|---|---|---|
| Temperature | 45° C. | | | |
| Autosampler temperature | 15° C. | | | |
| Mobile phase | A: 0.1% formic acid-water | | | |
| | B: 0.1% formic acid-acetonitrile | | | |
| | | | mobile | mobile |
| | | flow | phase | phase |
| | time(min) | rate(ml/min) | A (%) | B (%) |
| Mobile phase | 0.10 | 0.4 | 72~75 | 20~25 |
| gradient | 1.50 | 0.4 | 65~68 | 30~35 |
| | 2.80 | 0.4 | 65~68 | 30~35 |
| | 2.81 | 0.4 | 58~62 | 40~45 |
| | 4.00 | 0.4 | 58~62 | 40~45 |
| | 6.00 | 0.4 | 38~42 | 60~65 |
| | 7.00 | 0.4 | 18~25 | 80~85 |

| | |
|---|---|
| Autosampler wash | 80% formic acid |
| Sample size | 15 μL |
| Pre-balance before | inject the wash solution once |
| sample injection | |

### III. Parameters for Mass Spectrum

TABLE 9
details of ion source parameters under positive and negative modes.
Turbo Ion spray (ESI+)

| Curtain | Collision | Ion Spray | Tem- | | |
|---|---|---|---|---|---|
| Gas | Gas | Voltage | perature | Ion Source | Ion Source |
| (CUR) | (CAD) | (IS) | (TEM) | Gas1(Gas1) | Gas2(Gas2) |
| 30 | 8 | 5200 | 500 | 45 | 45 |

Turbo Ion spray (ESI−)

| Curtain | Collision | Ion Spray | Tem- | | |
|---|---|---|---|---|---|
| Gas | Gas | Voltage | perature | Ion Source | Ion Source |
| (CUR) | (CAD) | (IS) | (TEM) | Gas1(Gas1) | Gas2(Gas2) |
| 30 | 8 | −4500 | 500 | 50 | 50 |

TABLE 10
Scheduled MRM under positive and negative modes.
Turbo Ion spray (ESI+)

| | | | |
|---|---|---|---|
| MRM detection | 60 sec | Target Scan Time | 0.4 sec |

Turbo Ion spray (ESI−)

| | | | |
|---|---|---|---|
| MRM detection | 60 sec | Target Scan Time | 0.4 sec |

### 5. Quality Control

### QC Sample

Equal volume (15 ul) of serum derived from each individual from this cohort was pooled together, and the pooled sample was used as the QC sample. At least 5 QC samples were arranged in each detection batch. Peak areas of metabolites for all individuals were normalized to the same QC sample before subsequent analysis.

### Positive and Negative Reference Samples

Equal volume of serum from normal or advanced CRC individuals was pooled retrospectively as the negative-pool and positive-pool (total volume of 10 ml for each pool sample). One positive and one negative reference sample were arranged into each batch.

### Internal Standard

13C-Cholic Acid was used as the internal standard. 10 μL internal standard solution (5 μg/mL 13C-Cholic Acid) was added into each sample before metabolites extraction. CV/RSD of the internal standard was used to evaluate the precision of metabolomics detection, which should be <15%.

### 6. Data Analysis

Data preprocessing, statistical analysis, and predictive model building were conducted using R programming (v4.2.1).

### 7. Statistical Analysis

Relative abundances of metabolites for each individual were normalized to the same QC sample using the LOESS (locally weighted regression) algorithm, and were used for all subsequent analyses.

Serum metabolites that exhibit significant differentiation between the positive group (CRC and advanced adenoma individuals) and negative group (normal, colorectal polyps, low-risk adenoma individuals) were selected based on the following parameters:
First, biomarkers should be reliably tested. Thus, only metabolites with CV (Coefficient of Variation) value in all QC samples less than 15% were filtered out and used for subsequent feature selection for the construction of the diagnostic model;
Afterwards, metabolites that meet either parament I or parameter II were all filtered for subsequent use.

Parament I: based on the Analysis of variance (ANOVA) test, metabolites with a p-value of <0.05 in the training cohort were defined as significantly altered and were selected.

Parament II: performances of predicting models were evaluated based on single metabolites or a combination of two metabolites. Metabolites involved in either model with AUC>0.55 were also selected and used for further model construction.

### 8. Selection of the Metabolites for Detecting the Colorectal Cancer and Precancerous Lesions

To select the metabolite features for the CRC panel, the LASSO algorithm was implemented with 10-fold cross-validation for feature selection from the serum metabolomics data. The selected feature was subsequently used to construct a prediction model by logic regression in the training cohort, and the cut-off value was set at the point to achieve the highest accuracy.

9. The Training and Testing Cohort Used in this Study

Based on the study population described in the method part, these individuals were randomly divided into the training cohort and the testing cohort specifically, at a ratio of 7:3. As shown in the table below, the training set includes 494 individuals. This cohort was used to construct a prediction model and determine the cut-off value to determine positive vs, negative readouts. Among them, 165 individuals (from normal to low-risk adenoma) were classified into the negative group, while 329 individuals (advanced adenoma and CRC) were classified into the positive group. The age and gender of different groups and stages of CRC patients were also listed.

Similarly, as is shown in the table below, 207 individuals were involved in the testing cohort. This cohort was used to further test the clinical performances of the model and cut-off value determined from the training cohort. Within this cohort, 69 individuals were assigned to the negative group, while 138 individuals were enrolled in the positive group.

TABLE 11
Composition and basic information of the training cohort and the testing
cohort.

| Training | | Colorectal | Low-risk | Advanced | |
|---|---|---|---|---|---|
| Cohort | Normal | polyps | adenoma | adenoma | CRC |
| Number | 130 | 15 | 20 | 59 | 270 |
| Gender | M:64/ | M:11/F:4 | M:15/F:5 | M:40/F:19 | M:163/ |
| (Male/ | F:66 | | | | F:107 |
| Female) | | | | | |
| Age | 56.7 ± 7.93 | 57.5 ± 10.28 | 62.5 ± 7.34 | 61.6 ± 8.38 | 63.0 ± 8.9 |
| (mean ± SD) | | | | | |
| CRC stage | | | | | |
| Stage 0 | / | / | / | / | 8 |
| Stage I | / | / | / | / | 53 |
| Stage II | / | / | / | / | 100 |
| Stage III | / | / | / | / | 95 |
| Stage IV | / | / | / | / | 14 |
| Testing | | Colorectal | Low-risk | Advanced | |
| cohort | Normal | polyps | adenoma | adenoma | CRC |
| Number | 55 | 6 | 8 | 24 | 114 |
| Gender | M:25/ | M:4/F:2 | M:5/F:3 | M:17/F:7 | M:67/ |
| (Male/ | F:30 | | | | F:47 |
| Female) | | | | | |
| Age | 53 ± 7.07 | 57.8 ± 12.27 | 61 ± 11.01 | 61.5 ± 8.8 | 61.7 ± 9.5 |
| (mean ± SD) | | | | | |
| CRC stage | | | | | |
| Stage 0 | / | / | / | / | 5 |
| Stage I | / | / | / | / | 20 |
| Stage II | / | / | / | / | 45 |
| Stage III | / | / | / | / | 40 |
| Stage IV | / | / | / | / | 4 |

### Example 1 Targeted Metabolomics Detection and Feature Selection for Predicting Models

### I. Targeted Metabolomics Detection of Candidate Metabolites

A panel of 368 serum metabolites that showed potential for discriminating colorectal cancer and advanced adenoma from normal and low-risk polyps individuals were established, and their transitions were acquired based on MRM detection with the 4500MD UPLC-MS system. To further select serum metabolites that exhibit significant differences between the positive group (CRC and advanced adenoma individuals) and negative group (normal, colorectal polyps, low-risk adenoma individuals), targeted metabolomics detection of the above 368 metabolites panel was carried out within the training cohort, and feature selection was carried out based on the parameters described at Statistical analysis section.

As is shown in table 12 below, based on the parameter I, 208 metabolites were filtered, and used for subsequent model construction.

TABLE 12
CV % values, fold changes, and ANOVA p values between positive vs. negative groups for
each metabolite.

| | Fold | ANOVA | | Fold | ANOVA | | Fold |
|---|---|---|---|---|---|---|---|
| | change | p-value | | change | p-value | | change |
| | (positive | (Positive | CV (%) of | (positive | (Positive | CV (%) of | (positive |
| | vs. | vs. | all QC | vs. | vs. | all QC | vs. |
| Meta ID | negative) | negative) | samples | negative) | negative) | samples | negative) |
| BN001 | 2.64 | 8.20E−03 | 3.18 | X433 | 1.16 | 4.67E−02 | 4.04 |
| BN002 | 1.7 | 4.37E−04 | 12.57 | X434 | 1.3 | 9.37E−05 | 4.45 |
| BN003 | 2.25 | 5.95E−03 | 7.02 | X435 | 1.18 | 3.15E−02 | 6.22 |
| BN005 | 2 | 2.74E−04 | 2.17 | X436 | 1.19 | 1.55E−02 | 4.41 |
| BN006 | 1.34 | 3.25E−03 | 0.72 | X437 | 1.23 | 3.54E−03 | 6 |
| BN015 | 0.81 | 2.42E−13 | 2.22 | X448 | 1.26 | 9.85E−04 | 6.81 |
| BN016 | 1.24 | 5.02E−04 | 3.03 | X449 | 0.97 | 4.67E−02 | 6.93 |
| BN017 | 0.88 | 2.31E−03 | 0.54 | X451 | 0.98 | 2.61E−02 | 3.77 |
| BN018 | 1.27 | 7.15E−04 | 14.54 | X454 | 0.92 | 1.02E−03 | 6.64 |
| BN028 | 1.18 | 1.83E−02 | 5.4 | X455 | 0.97 | 4.44E−02 | 8.6 |
| BN029 | 1.2 | 1.31E−02 | 1.51 | X456 | 0.84 | 4.84E−03 | 6.07 |
| BN030 | 1.11 | 1.49E−03 | 3.89 | X457 | 0.76 | 2.32E−11 | 12.2 |
| BN031 | 1.06 | 4.83E−02 | 5.78 | X458 | 1.15 | 6.09E−05 | 7.47 |
| BN032 | 1.84 | 3.83E−02 | 7.96 | X463 | 1.19 | 1.09E−02 | 5.73 |
| BN034 | 1.09 | 4.17E−03 | 3.03 | X470 | 0.82 | 9.63E−04 | 5.59 |
| BN035 | 1.28 | 2.43E−03 | 8.69 | X471 | 0.86 | 1.19E−02 | 8.59 |
| BP001 | 0.66 | 2.38E−02 | 6.6 | X473 | 0.66 | 1.17E−17 | 11.51 |
| BP006 | 1.1 | 1.75E−02 | 6.31 | X508 | 0.62 | 2.44E−03 | 3.38 |
| BP010 | 1.14 | 6.88E−03 | 2.67 | X509 | 0.7 | 5.76E−05 | 2.7 |
| BP013 | 1.32 | 5.23E−04 | 4.2 | X511 | 0.92 | 1.63E−02 | 6.58 |
| C001 | 1.15 | 1.55E−04 | 4.08 | X512 | 0.54 | 1.77E−06 | 6.87 |
| C005 | 1.06 | 1.03E−02 | 3.68 | X513 | 1.18 | 1.77E−05 | 8.54 |
| C009 | 1.11 | 2.69E−07 | 4.15 | X515 | 1.24 | 3.89E−06 | 6.9 |
| C011 | 1.21 | 3.97E−09 | 2.43 | X517 | 1.19 | 9.24E−03 | 2.06 |
| C012 | 1.12 | 3.07E−03 | 4.22 | X519 | 0.69 | 1.07E−14 | 6.52 |
| C014 | 1.29 | 2.42E−03 | 10.8 | X523 | 0.9 | 1.94E−02 | 2.93 |
| C016 | 0.89 | 2.36E−02 | 2.63 | X525 | 0.66 | 4.48E−06 | 10.07 |
| C017 | 0.71 | 1.47E−09 | 2.32 | X531 | 1.07 | 1.16E−02 | 7.83 |
| C019 | 0.55 | 3.34E−15 | 2.14 | X535 | 0.76 | 9.80E−04 | 7.53 |
| C020 | 0.72 | 3.21E−03 | 1.92 | X536 | 0.61 | 6.94E−09 | 5.35 |
| C022 | 1.17 | 1.75E−04 | 7.74 | X540 | 0.78 | 4.80E−14 | 2.29 |
| C023 | 1.67 | 6.18E−09 | 2.61 | X541 | 1.24 | 5.32E−03 | 5.94 |
| C027 | 0.55 | 1.97E−13 | 2.64 | X542 | 0.61 | 8.19E−07 | 5.04 |
| C028 | 1.06 | 1.26E−02 | 2.9 | X543 | 1.16 | 2.20E−02 | 5.87 |
| C029 | 1.38 | 6.88E−10 | 2.6 | X546 | 1.17 | 3.97E−02 | 6.18 |
| C030 | 1.1 | 3.72E−02 | 3.39 | X547 | 1.17 | 4.37E−02 | 8.98 |
| C035 | 0.93 | 3.11E−03 | 1.44 | X549 | 1.23 | 3.14E−03 | 6.12 |
| C036 | 1.19 | 2.37E−04 | 3.07 | X550 | 1.35 | 1.12E−02 | 12.09 |
| C037 | 0.43 | 5.74E−05 | 2.38 | X555 | 1.2 | 9.23E−03 | 3.05 |
| C038 | 1.68 | 4.69E−08 | 2.94 | X556 | 1.15 | 3.85E−02 | 1.52 |
| C040 | 0.6 | 1.39E−09 | 14.74 | X557 | 1.22 | 7.18E−03 | 8.62 |
| C041 | 0.88 | 4.86E−02 | 2.93 | X558 | 0.82 | 2.79E−07 | 9.35 |
| C102 | 0.39 | 5.54E−10 | 8.38 | X559 | 1.18 | 9.59E−03 | 9.28 |
| C108 | 0.63 | 2.51E−04 | 3.54 | X560 | 0.8 | 5.09E−13 | 2.8 |
| C110 | 0.86 | 4.95E−02 | 6.29 | X561 | 0.8 | 3.93E−12 | 4.14 |
| C112 | 0.86 | 5.41E−03 | 5.43 | X563 | 1.27 | 5.30E−04 | 9.21 |
| C115 | 1.22 | 1.00E−03 | 3.82 | X565 | 0.77 | 2.20E−13 | 12.58 |
| C121 | 0.74 | 6.53E−04 | 1.03 | X566 | 0.79 | 3.76E−14 | 4.33 |
| C122 | 1.1 | 2.61E−02 | 3 | X567 | 0.79 | 1.60E−12 | 6.73 |
| C124 | 1.2 | 4.32E−05 | 1.67 | X569 | 0.79 | 6.26E−14 | 5.48 |
| C127 | 1.11 | 1.71E−03 | 1.11 | X570 | 0.8 | 1.71E−12 | 3.11 |
| C129 | 1.18 | 9.15E−04 | 1.24 | X571 | 0.79 | 1.66E−12 | 3.87 |
| C130 | 1.13 | 2.67E−02 | 3.24 | X572 | 0.8 | 1.10E−11 | 2.82 |
| C131 | 0.81 | 5.69E−09 | 1 | X573 | 0.78 | 2.66E−17 | 2.16 |
| C135 | 0.38 | 2.35E−06 | 7.92 | X574 | 0.78 | 2.65E−17 | 3.57 |
| C136 | 0.88 | 3.76E−02 | 7.28 | X575 | 0.77 | 2.15E−17 | 4.66 |
| C144 | 0.86 | 5.81E−03 | 2.57 | X576 | 0.78 | 1.63E−15 | 3.19 |
| C145 | 0.89 | 1.10E−02 | 7.92 | X577 | 1.19 | 1.72E−02 | 2.27 |
| C146 | 0.93 | 1.42E−02 | 0.54 | X578 | 0.78 | 2.45E−16 | 2.54 |
| C148 | 0.91 | 1.02E−02 | 2.12 | X579 | 0.79 | 3.68E−13 | 9.77 |
| C150 | 0.76 | 3.62E−06 | 11.31 | X580 | 0.79 | 2.20E−14 | 4.6 |
| DS11 | 0.82 | 8.44E−03 | 2.19 | X581 | 0.78 | 1.94E−15 | 1.37 |
| X004 | 0.59 | 1.32E−10 | 1.99 | X582 | 1.4 | 2.22E−04 | 7.57 |
| X006 | 1.58 | 1.13E−05 | 4.72 | X583 | 0.79 | 1.40E−14 | 3.11 |
| X013 | 0.7 | 1.83E−04 | 1.45 | X584 | 0.79 | 1.66E−13 | 11.71 |
| X016 | 1.36 | 1.88E−03 | 4.01 | X585 | 0.79 | 6.52E−13 | 3.18 |
| X023 | 0.87 | 2.00E−03 | 4.57 | X586 | 0.79 | 3.09E−14 | 3.81 |
| X024 | 0.87 | 1.51E−02 | 2.27 | X587 | 0.79 | 5.06E−15 | 4.4 |
| X032 | 1.09 | 2.85E−02 | 7.52 | X588 | 0.79 | 2.64E−13 | 5.49 |
| X036 | 1.47 | 1.88E−06 | 2.58 | X589 | 0.77 | 4.30E−16 | 5.81 |
| X042 | 1.08 | 9.24E−03 | 0.93 | X591 | 0.79 | 2.47E−14 | 2.85 |
| X043 | 2.13 | 5.63E−08 | 3.23 | X592 | 0.78 | 2.64E−16 | 0.59 |
| X055 | 0.89 | 1.58E−03 | 2.32 | X593 | 0.8 | 3.74E−13 | 4.31 |
| X068 | 1.63 | 2.87E−03 | 4.15 | X595 | 0.78 | 1.13E−16 | 2.5 |
| X070 | 1.45 | 3.48E−07 | 10.54 | X596 | 0.79 | 3.03E−14 | 9.37 |
| X082 | 58.1 | 1.34E−04 | 5.96 | X597 | 0.8 | 4.05E−13 | 4.62 |
| X152 | 0.79 | 1.03E−03 | 0.8 | X598 | 0.79 | 7.96E−13 | 6.55 |
| X155 | 1.35 | 1.53E−03 | 1.22 | X599 | 0.79 | 8.86E−16 | 1.87 |
| X166 | 0.81 | 5.34E−14 | 2.06 | X600 | 0.79 | 4.16E−12 | 11.82 |
| X183 | 0.8 | 7.41E−09 | 5.38 | X601 | 0.79 | 4.72E−15 | 2.82 |
| X278 | 0.7 | 3.29E−17 | 4.68 | X602 | 0.78 | 8.01E−16 | 4.85 |
| X280 | 0.7 | 1.15E−07 | 2.09 | X603 | 0.92 | 1.83E−05 | 5.95 |
| X281 | 0.57 | 3.34E−14 | 3.29 | X605 | 0.77 | 1.31E−18 | 2.94 |
| X285 | 0.8 | 1.34E−09 | 2.28 | X606 | 0.88 | 2.86E−02 | 3.16 |
| X286 | 0.79 | 7.26E−10 | 13.98 | X608 | 0.76 | 3.66E−03 | 3.51 |
| X289 | 0.92 | 2.49E−02 | 1.74 | X609 | 0.8 | 6.04E−16 | 3.49 |
| X292 | 1.42 | 1.71E−04 | 1.31 | X614 | 0.85 | 5.93E−03 | 8.6 |
| X293 | 0.87 | 6.94E−05 | 1.68 | X616 | 0.81 | 3.27E−03 | 2.25 |
| X314 | 0.63 | 4.37E−19 | 4.12 | X617 | 0.84 | 3.59E−03 | 9.52 |
| X402 | 1.08 | 7.68E−03 | 2.29 | X618 | 1.35 | 8.15E−04 | 8.73 |
| X403 | 0.61 | 2.42E−11 | 2.45 | X619 | 0.89 | 9.84E−03 | 6.43 |
| X405 | 1.05 | 3.32E−02 | 2.22 | X620 | 0.88 | 3.49E−02 | 2.2 |
| X407 | 0.48 | 2.35E−04 | 6.3 | X621 | 0.82 | 5.04E−03 | 4.58 |
| X409 | 0.71 | 1.70E−10 | 9.35 | X624 | 1.33 | 3.24E−02 | 6.46 |
| X412 | 1.11 | 6.08E−07 | 6.36 | X628 | 1.34 | 2.17E−02 | 7.22 |
| X415 | 1.68 | 3.41E−08 | 3.51 | X629 | 0.87 | 1.19E−02 | 10.36 |
| X419 | 1.18 | 1.19E−02 | 4.23 | X633 | 0.82 | 1.25E−02 | 4.2 |
| X426 | 1.21 | 5.71E−03 | 3.46 | X634 | 0.82 | 9.56E−03 | 4.19 |
| X429 | 0.88 | 1.93E−02 | 0.75 | X635 | 0.85 | 4.89E−02 | 10.77 |
| X431 | 1.15 | 2.93E−02 | 8.51 | X639 | 0.86 | 7.47E−10 | 1.39 |
| X432 | 1.16 | 4.78E−02 | 2.72 | X010 | 3.88 | 9.67E−10 | 6.43 |
| X012 | 2.49 | 3.88E−10 | 11.73 | X133 | 3.72 | 1.62E−10 | 12.45 |
| X139 | 2.04 | 2.68E−08 | 5.38 | X141 | 1.90 | 1.27E−09 | 3.45 |
| X144 | 2.33 | 1.69E−15 | 5.62 | X196 | 4.97 | 1.60E−10 | 8.89 |

The term “Fold change” refers to a parameter describing how much a quantity changes between an original and a subsequent measurement. The term “P-value” refers to the probability, under the null hypothesis about the unknown distribution of the test statistic, to have observed a value as extreme or more extreme than the value actually observed.

Based on the parameter II, the performances (AUC) of prediction models are evaluated based on either a single metabolite or a combination of two metabolites. 155 metabolites fulfilled the parameter II and were filtered.

TABLE 13
CV % values, fold changes, ANOVA p values and AUC values of
prediction models between positive vs. negative groups based
on each metabolite or combination of two metabolites.

| | | | AUC | | |
|---|---|---|---|---|---|
| | | | of the | | |
| | | | pre- | | |
| | Fold | | diction | | |
| | change | | model | | CV |
| | (posi- | ANOVA | based | | (%) |
| | tive | p-value | on a | AUC of the prediction | of all |
| | vs. | (Positive | single | model based on the | QC |
| Meta | nega- | vs. | meta- | combination of two | sam- |
| ID | tive) | negative) | bolite | metabolite | ples |
| BN004 | 1.12 | 9.7E−01 | 0.53 | AUC(BN004 + DS01) = | 5.9 |
| | | | | 0.587 | |
| BN010 | 1.1 | 3.52E−01 | 0.5 | AUC(BN010 + C004) = | 2.98 |
| | | | | 0.555 | |
| BN011 | 0.97 | 7.59E−01 | 0.51 | AUC(BN011 + DS01) = | 3.11 |
| | | | | 0.569 | |
| BN012 | 0.89 | 1.94E−01 | 0.57 | | 2.88 |
| BN019 | 1.05 | 4.30E−01 | 0.51 | AUC(BN019 + C004) = | 8.02 |
| | | | | 0.576 | |
| BN020 | 1.2 | 1.55E−01 | 0.53 | AUC(BN020 + C004) = | 3.17 |
| | | | | 0.578 | |
| BN021 | 0.97 | 8.63E−01 | 0.49 | AUC(BN021 + X554) = | 4.25 |
| | | | | 0.55 | |
| BN022 | 0.99 | 9.63E−01 | 0.5 | AUC(BN022 + DS01) = | 6.82 |
| | | | | 0.558 | |
| BN023 | 0.97 | 6.78E−01 | 0.52 | AUC(BN023 + DS01) = | 3.86 |
| | | | | 0.554 | |
| BN025 | 1.05 | 1.40E−01 | 0.54 | AUC(BN025 + C004) = | 3.44 |
| | | | | 0.586 | |
| BN027 | 1.05 | 1.33E−01 | 0.51 | AUC(BN027 + C004) = | 2.84 |
| | | | | 0.574 | |
| BP002 | 1.1 | 6.15E−01 | 0.58 | / | 0.86 |
| BP003 | 1.13 | 5.39E−02 | 0.59 | / | 7.65 |
| BP007 | 1.07 | 1.04E−01 | 0.53 | AUC(BP007 + C004) = | 7.94 |
| | | | | 0.574 | |
| BP008 | 0.93 | 9.97E−02 | 0.54 | AUC(BP008 + C004) = | 0.83 |
| | | | | 0.588 | |
| BP009 | 1.1 | 2.13E−01 | 0.56 | / | 9.3 |
| BP011 | 1.03 | 5.92E−01 | 0.52 | AUC(BP011 + C004) = | 1.27 |
| | | | | 0.584 | |
| BP012 | 1.01 | 8.52E−01 | 0.54 | AUC(BP012 + DS01) = | 1.8 |
| | | | | 0.57 | |
| C002 | 0.81 | 2.63E−01 | 0.56 | / | 1.54 |
| C004 | 1.77 | 7.43E−02 | 0.53 | AUC(C004 + X019) = | 1.85 |
| | | | | 0.593 | |
| C006 | 1.06 | 1.10E−01 | 0.54 | AUC(C006 + X475) = | 2.46 |
| | | | | 0.565 | |
| C008 | 1.01 | 7.58E−01 | 0.48 | AUC(C004 + C008) = | 3.16 |
| | | | | 0.554 | |
| C010 | 1.45 | 8.92E−02 | 0.55 | / | 7.55 |
| C013 | 0.98 | 6.82E−01 | 0.5 | AUC(C013 + DS01) = | 3.9 |
| | | | | 0.552 | |
| C015 | 1.63 | 2.39E−01 | 0.56 | / | 5.29 |
| C021 | 1.05 | 1.40E−01 | 0.51 | AUC(C021 + X611) = | 3.63 |
| | | | | 0.563 | |
| C024 | 0.91 | 9.77E−02 | 0.55 | / | 1.06 |
| C025 | 1.02 | 4.28E−01 | 0.51 | AUC(C025 + X066) = | 2.18 |
| | | | | 0.555 | |
| C026 | 1.04 | 1.90E−01 | 0.51 | AUC(C026 + X611) = | 2.45 |
| | | | | 0.569 | |
| C031 | 0.98 | 7.86E−02 | 0.55 | / | 1.05 |
| C032 | 1.04 | 1.83E−01 | 0.51 | AUC(C032 + X611) = | 2.3 |
| | | | | 0.572 | |
| C033 | 0.97 | 3.82E−01 | 0.53 | AUC(C033 + X627) = | 1.03 |
| | | | | 0.557 | |
| C034 | 0.97 | 3.20E−01 | 0.53 | AUC(C034 + X611) = | 1.81 |
| | | | | 0.571 | |
| C042 | 0.91 | 5.14E−01 | 0.5 | AUC(C042 + C132) = | 11.26 |
| | | | | 0.551 | |
| C043 | 1.12 | 3.65E−01 | 0.51 | AUC(C043 + C105) = | 9.65 |
| | | | | 0.554 | |
| C105 | 0.94 | 1.07E−01 | 0.54 | AUC(C105 + C128) = | 8.79 |
| | | | | 0.569 | |
| C113 | 1.02 | 6.39E−01 | 0.52 | AUC(C113 + C132) = | 8.45 |
| | | | | 0.555 | |
| C114 | 1.01 | 8.84E−01 | 0.52 | AUC(C114 + DS01) = | 3.07 |
| | | | | 0.559 | |
| C116 | 0.99 | 8.89E−01 | 0.54 | AUC(C116 + X154) = | 3.12 |
| | | | | 0.563 | |
| C118 | 0.67 | 1.27E−01 | 0.5 | AUC(C118 + X562) = | 9.39 |
| | | | | 0.557 | |
| C119 | 1.02 | 6.01E−01 | 0.5 | AUC(C119 + X594) = | 5.09 |
| | | | | 0.554 | |
| C120 | 1.73 | 2.20E−01 | 0.56 | / | 1.36 |
| C128 | 1.05 | 5.26E−02 | 0.54 | AUC(C128 + C132) = | 0.57 |
| | | | | 0.573 | |
| C132 | 0.97 | 2.18E−01 | 0.55 | AUC(C132 + C149) = | 1 |
| | | | | 0.613 | |
| C134 | 1.01 | 6.78E−01 | 0.51 | AUC(C134 + X446) = | 2.95 |
| | | | | 0.56 | |
| C137 | 1.2 | 1.61E−01 | 0.49 | AUC(C004 + C137) = | 2.65 |
| | | | | 0.551 | |
| C138 | 0.68 | 1.04E−01 | 0.53 | AUC(C138 + DS01) = | 3.44 |
| | | | | 0.565 | |
| C139 | 1.04 | 2.49E−01 | 0.51 | AUC(C139 + X594) = | 5.67 |
| | | | | 0.562 | |
| C140 | 1.04 | 1.45E−01 | 0.52 | AUC(C140 + X594) = | 10.05 |
| | | | | 0.566 | |
| C141 | 1.66 | 2.03E−01 | 0.57 | / | 3.85 |
| C142 | 1.23 | 2.88E−01 | 0.53 | AUC(C142 + X590) = | 9.79 |
| | | | | 0.561 | |
| C143 | 0.99 | 9.47E−01 | 0.52 | AUC(C143 + DS02) = | 3.21 |
| | | | | 0.56 | |
| C147 | 0.99 | 7.56E−01 | 0.53 | AUC(C147 + C149) = | 2.52 |
| | | | | 0.584 | |
| C149 | 1.06 | 9.14E−02 | 0.51 | AUC(C149 + X066) = | 1.34 |
| | | | | 0.595 | |
| DS01 | 1.37 | 2.08E−01 | 0.54 | AUC(DS01 + X532) = | 3.93 |
| | | | | 0.575 | |
| DS02 | 1.14 | 3.26E−01 | 0.53 | AUC(DS02 + X590) = | 2.18 |
| | | | | 0.561 | |
| DS03 | 0.99 | 9.08E−01 | 0.51 | AUC(C004 + DS03) = | 3.24 |
| | | | | 0.564 | |
| DS04 | 0.96 | 9.22E−01 | 0.52 | AUC(DS01 + DS04) = | 5.08 |
| | | | | 0.554 | |
| DS05 | 1.14 | 3.14E−01 | 0.51 | AUC(DS05 + X066) = | 0.83 |
| | | | | 0.553 | |
| DS06 | 1.08 | 5.71E−01 | 0.48 | AUC(DS06 + X594) = | 1.34 |
| | | | | 0.55 | |
| DS07 | 1.2 | 2.28E−01 | 0.49 | AUC(DS07 + X604) = | 1.94 |
| | | | | 0.557 | |
| DS08 | 0.88 | 4.11E−01 | 0.5 | AUC(C004 + DS08) = | 3.12 |
| | | | | 0.553 | |
| DS09 | 0.29 | 2.51E−01 | 0.56 | / | 2.62 |
| DS10 | 0.81 | 5.26E−01 | 0.49 | AUC(DS10 + X564) = | 3.97 |
| | | | | 0.551 | |
| X011 | 1.05 | 4.78E−01 | 0.48 | AUC(C004 + X011) = | 3.33 |
| | | | | 0.559 | |
| X019 | 1.11 | 2.01E−01 | 0.53 | AUC(X019 + X446) = | 3.45 |
| | | | | 0.593 | |
| X051 | 1.04 | 3.16E−01 | 0.52 | AUC(X051 + X066) = | 3.41 |
| | | | | 0.587 | |
| X066 | 0.96 | 1.24E−01 | 0.55 | AUC(X066 + X518) = | 1.79 |
| | | | | 0.568 | |
| X154 | 1.05 | 6.00E−01 | 0.49 | AUC(C116 + X154) = | 4.44 |
| | | | | 0.563 | |
| X160 | 1.03 | 5.91E−01 | 0.52 | AUC(X160 + X590) = | 2.88 |
| | | | | 0.551 | |
| X178 | 1.02 | 9.15E−01 | 0.55 | / | 4.33 |
| X188 | 0.91 | 1.37E−01 | 0.56 | / | 0.95 |
| X401 | 0.9 | 6.95E−02 | 0.53 | AUC(X401 + X544) = | 4.97 |
| | | | | 0.572 | |
| X408 | 0.95 | 5.43E−01 | 0.51 | AUC(X408 + X518) = | 4.28 |
| | | | | 0.551 | |
| X411 | 1.03 | 7.01E−01 | 0.49 | AUC(X411 + X562) = | 5.91 |
| | | | | 0.553 | |
| X416 | 1.35 | 5.63E−02 | 0.54 | AUC(X416 + X594) = | 3.03 |
| | | | | 0.568 | |
| X421 | 1.07 | 3.22E−01 | 0.54 | AUC(X421 + X450) = | 2.05 |
| | | | | 0.629 | |
| X422 | 1.05 | 4.35E−01 | 0.53 | AUC(X422 + X450) = | 5.59 |
| | | | | 0.619 | |
| X423 | 0.99 | 8.68E−01 | 0.49 | AUC(X423 + X464) = | 4.05 |
| | | | | 0.633 | |
| X424 | 1.02 | 8.42E−01 | 0.53 | AUC(X424 + X464) = | 9.55 |
| | | | | 0.569 | |
| X425 | 1.11 | 6.22E−02 | 0.54 | AUC(X425 + X446) = | 3.41 |
| | | | | 0.609 | |
| X427 | 0.97 | 6.56E−01 | 0.48 | AUC(X427 + X604) = | 3.85 |
| | | | | 0.593 | |
| X428 | 1.17 | 5.33E−02 | 0.53 | AUC(X428 + X532) = | 5.59 |
| | | | | 0.574 | |
| X430 | 1.09 | 1.56E−01 | 0.54 | AUC(X430 + X450) = | 4.47 |
| | | | | 0.643 | |
| X438 | 1.02 | 8.01E−01 | 0.51 | AUC(X438 + X446) = | 3.53 |
| | | | | 0.643 | |
| X439 | 0.99 | 8.32E−01 | 0.5 | AUC(X439 + X446) = | 5.7 |
| | | | | 0.616 | |
| X440 | 1.09 | 1.75E−01 | 0.54 | AUC(X440 + X450) = | 6.31 |
| | | | | 0.643 | |
| X441 | 1.02 | 8.13E−01 | 0.51 | AUC(X441 + X444) = | 7.63 |
| | | | | 0.626 | |
| X442 | 0.94 | 1.97E−01 | 0.52 | AUC(X442 + X594) = | 14.85 |
| | | | | 0.556 | |
| X443 | 0.96 | 4.33E−01 | 0.52 | AUC(X443 + X446) = | 6.55 |
| | | | | 0.633 | |
| X444 | 0.96 | 5.46E−01 | 0.51 | AUC(X444 + X464) = | 9.92 |
| | | | | 0.604 | |
| X445 | 1.06 | 3.32E−01 | 0.54 | AUC(X445 + X450) = | 6.66 |
| | | | | 0.655 | |
| X446 | 0.91 | 9.73E−02 | 0.54 | AUC(X446 + X465) = | 6.08 |
| | | | | 0.638 | |
| X447 | 0.98 | 4.14E−01 | 0.52 | AUC(X447 + X518) = | 13.48 |
| | | | | 0.553 | |
| X450 | 0.93 | 2.07E−01 | 0.51 | AUC(X450 + X465) = | 7.53 |
| | | | | 0.671 | |
| X452 | 0.98 | 2.28E−01 | 0.52 | AUC(C006 + X452) = | 11.94 |
| | | | | 0.557 | |
| X453 | 0.99 | 2.71E−01 | 0.53 | AUC(X453 + X518) = | 3.79 |
| | | | | 0.553 | |
| X459 | 0.98 | 6.95E−01 | 0.5 | AUC(X459 + X562) = | 6.39 |
| | | | | 0.588 | |
| X460 | 1.23 | 5.07E−01 | 0.47 | AUC(C132 + X460) = | 7.61 |
| | | | | 0.55 | |
| X461 | 1.12 | 6.46E−02 | 0.54 | AUC(X461 + X475) = | 3.24 |
| | | | | 0.586 | |
| X462 | 1.19 | 7.15E−01 | 0.5 | AUC(X462 + X532) = | 4.45 |
| | | | | 0.561 | |
| X464 | 1.12 | 9.74E−02 | 0.54 | AUC(X464 + X607) = | 3.95 |
| | | | | 0.628 | |
| X465 | 1.07 | 2.48E−01 | 0.54 | AUC(X465 + X607) = | 7.69 |
| | | | | 0.634 | |
| X466 | 1.03 | 6.15E−01 | 0.52 | AUC(X466 + X607) = | 7.82 |
| | | | | 0.621 | |
| X467 | 1.02 | 8.00E−01 | 0.52 | AUC(X467 + X475) = | 8.3 |
| | | | | 0.619 | |
| X468 | 1.01 | 8.06E−01 | 0.52 | AUC(X468 + X475) = | 5.93 |
| | | | | 0.621 | |
| X469 | 1.1 | 1.07E−01 | 0.54 | AUC(X469 + X475) = | 7.18 |
| | | | | 0.613 | |
| X472 | 1.04 | 4.81E−01 | 0.53 | AUC(X472 + X607) = | 7.93 |
| | | | | 0.613 | |
| X474 | 1.04 | 5.57E−01 | 0.52 | AUC(X474 + X475) = | 11.44 |
| | | | | 0.603 | |
| X475 | 0.91 | 1.04E−01 | 0.54 | AUC(X475 + X562) = | 5.14 |
| | | | | 0.629 | |
| X476 | 1.03 | 6.21E−01 | 0.52 | AUC(X476 + X607) = | 5.21 |
| | | | | 0.612 | |
| X477 | 1.01 | 8.32E−01 | 0.52 | AUC(X477 + X607) = | 10.81 |
| | | | | 0.595 | |
| X478 | 1.01 | 8.74E−01 | 0.52 | AUC(X478 + X607) = | 6.18 |
| | | | | 0.601 | |
| X479 | 1.02 | 7.89E−01 | 0.52 | AUC(X479 + X607) = | 13.4 |
| | | | | 0.586 | |
| X480 | 1.06 | 4.05E−01 | 0.52 | AUC(X480 + X607) = | 7.76 |
| | | | | 0.589 | |
| X516 | 1.05 | 1.24E−01 | 0.56 | / | 6.06 |
| X518 | 1.06 | 7.06E−02 | 0.55 | AUC(X518 + X611) = | 2.74 |
| | | | | 0.572 | |
| X532 | 0.97 | 5.82E−01 | 0.55 | AUC(X532 + X627) = | 2.48 |
| | | | | 0.602 | |
| X533 | 1.14 | 1.06E−01 | 0.52 | AUC(X533 + X590) = | 3.88 |
| | | | | 0.562 | |
| X534 | 1.17 | 5.16E−02 | 0.53 | AUC(X534 + X594) = | 6 |
| | | | | 0.562 | |
| X537 | 1.01 | 9.04E−01 | 0.52 | AUC(X537 + X590) = | 5.37 |
| | | | | 0.555 | |
| X538 | 0.99 | 9.37E−01 | 0.46 | AUC(X538 + X590) = | 6.25 |
| | | | | 0.558 | |
| X539 | 1.18 | 1.14E−01 | 0.56 | / | 6.73 |
| X544 | 1.12 | 9.65E−02 | 0.54 | AUC(X544 + X590) = | 3.53 |
| | | | | 0.607 | |
| X545 | 1.09 | 1.87E−01 | 0.51 | AUC(X545 + X548) = | 4.89 |
| | | | | 0.559 | |
| X548 | 1.16 | 5.62E−02 | 0.53 | AUC(X548 + X594) = | 4.15 |
| | | | | 0.562 | |
| X551 | 1.01 | 9.23E−01 | 0.52 | AUC(X551 + X562) = | 6.41 |
| | | | | 0.561 | |
| X552 | 1.11 | 8.64E−02 | 0.54 | AUC(X552 + X607) = | 7.28 |
| | | | | 0.581 | |
| X553 | 1.13 | 5.90E−02 | 0.55 | / | 2.23 |
| X554 | 1.11 | 8.95E−02 | 0.55 | AUC(X554 + X607) = | 2.8 |
| | | | | 0.631 | |
| X562 | 1.12 | 6.84E−02 | 0.55 | AUC(X562 + X607) = | 2.19 |
| | | | | 0.633 | |
| X564 | 1.12 | 9.45E−02 | 0.54 | AUC(X564 + X607) = | 8.68 |
| | | | | 0.626 | |
| X568 | 1.1 | 1.64E−01 | 0.54 | AUC(X568 + X607) = | 4.65 |
| | | | | 0.614 | |
| X590 | 0.9 | 2.28E−01 | 0.54 | AUC(X590 + X615) = | 12.13 |
| | | | | 0.586 | |
| X594 | 0.96 | 5.37E−02 | 0.54 | AUC(X594 + X611) = | 11.42 |
| | | | | 0.564 | |
| X604 | 1.11 | 1.02E−01 | 0.54 | AUC(X604 + X607) = | 3.9 |
| | | | | 0.612 | |
| X607 | 0.9 | 9.08E−02 | 0.53 | AUC(X607 + X615) = | 2.39 |
| | | | | 0.616 | |
| X610 | 1.12 | 8.36E−02 | 0.54 | AUC(X610 + X613) = | 7.94 |
| | | | | 0.579 | |
| X611 | 1.14 | 5.35E−02 | 0.55 | AUC(X611 + X613) = | 2.45 |
| | | | | 0.582 | |
| X612 | 1.05 | 4.21E−01 | 0.53 | AUC(X612 + X613) = | 10.83 |
| | | | | 0.588 | |
| X613 | 0.92 | 1.54E−01 | 0.53 | AUC(X613 + X636) = | 2.74 |
| | | | | 0.596 | |
| X615 | 1.06 | 3.50E−01 | 0.52 | AUC(X607 + X615) = | 10 |
| | | | | 0.616 | |
| X622 | 1.08 | 2.34E−01 | 0.53 | AUC(X622 + X631) = | 9.95 |
| | | | | 0.56 | |
| X623 | 1.05 | 4.32E−01 | 0.52 | AUC(X607 + X623) = | 9.17 |
| | | | | 0.607 | |
| X625 | 1.03 | 5.99E−01 | 0.51 | AUC(X625 + X626) = | 3.37 |
| | | | | 0.583 | |
| X626 | 1.05 | 4.15E−01 | 0.52 | AUC(X626 + X637) = | 2.69 |
| | | | | 0.556 | |
| X627 | 1.2 | 1.50E−01 | 0.53 | AUC(X532_X627) = | 11.32 |
| | | | | 0.602 | |
| X630 | 1.05 | 4.37E−01 | 0.53 | AUC(X630 + X637) = | 7.78 |
| | | | | 0.552 | |
| X631 | 1.03 | 6.06E−01 | 0.51 | AUC(X590 + X631) = | 13.16 |
| | | | | 0.577 | |
| X632 | 1.01 | 9.77E−01 | 0.53 | AUC(C004 + X632) = | 14.02 |
| | | | | 0.55 | |
| X636 | 1.03 | 6.80E−01 | 0.52 | AUC(X636 + X637) = | 5.92 |
| | | | | 0.554 | |
| X637 | 1 | 9.95E−01 | 0.51 | AUC(X554 + X637) = | 5.47 |
| | | | | 0.587 | |
| X638 | 1 | 9.56E−01 | 0.52 | AUC(C132 + X638) = | 7.83 |
| | | | | 0.55 | |
| X640 | 1.05 | 4.64E−01 | 0.52 | AUC(X607 + X640) = | 11.94 |
| | | | | 0.599 | |
| X641 | 1.13 | 9.95E−02 | 0.53 | AUC(X475 + X641) = | 11.83 |
| | | | | 0.585 | |

### II. Selection of Metabolites Involved in the Prediction Model

Based on these filtered serum metabolites described in the above 2 tables, a LASSO algorithm was performed with 10-fold cross-validation for feature selection from the serum metabolomics data of the training cohort to seek key metabolite biomarkers for detecting colorectal cancer and advanced adenoma. 81 metabolite features have been selected and used for subsequent model construction.

TABLE 14
CV % values, fold changes, ANOVA p values, and an average of raw/normalized
abundances between positive vs. negative groups for each metabolite.

| | | | | | | average | average |
|---|---|---|---|---|---|---|---|
| | | | | average | average | ratio in | ratio in |
| | | | | abundance | abundance | positive | negative |
| Meta | fold | ANOVA | CV | in positive | in negative | samples | samples |
| ID | change | p-value | (%) | samples | samples | (normalized) | (normalized) |
| BN001 | 2.64 | 8.20E−03 | 3.18 | 144244 | 119857 | 3.15E+01 | 1.19E+01 |
| BN002 | 1.7 | 4.37E−04 | 12.57 | 8451 | 7260 | 2.91E+00 | 1.71E+00 |
| BN003 | 2.25 | 5.95E−03 | 7.02 | 6299 | 5003 | 7.79E+00 | 3.47E+00 |
| BN004 | 1.12 | 9.7E−01 | 5.9 | 3552 | 3162 | 14.18E+00 | 13.02E+00 |
| BN005 | 2 | 2.74E−04 | 2.17 | 48697 | 40578 | 3.51E+00 | 1.76E+00 |
| BN006 | 1.34 | 3.25E−03 | 0.72 | 115145 | 89237 | 1.91E+00 | 1.45E+00 |
| BN010 | 1.1 | 3.52E−01 | 2.98 | 54673 | 47108 | 1.03E+00 | 9.13E−01 |
| BN011 | 0.97 | 7.59E−01 | 3.11 | 30298 | 31346 | 1.09E+00 | 1.13E+00 |
| BN015 | 0.81 | 2.42E−13 | 2.22 | 33522 | 32813 | 8.10E−01 | 1.01E+00 |
| BN016 | 1.24 | 5.02E−04 | 3.03 | 73630 | 71972 | 1.11E+00 | 8.90E−01 |
| BN017 | 0.88 | 2.31E−03 | 0.54 | 20067480 | 20408960 | 6.60E−01 | 7.50E−01 |
| BN018 | 1.27 | 7.15E−04 | 14.54 | 7813 | 8112 | 1.03E+00 | 8.10E−01 |
| BN029 | 1.2 | 1.31E−02 | 1.51 | 5697893 | 5690444 | 6.30E−01 | 5.30E−01 |
| BN030 | 1.11 | 1.49E−03 | 3.89 | 530253 | 515634 | 1.18E+00 | 1.07E+00 |
| BN031 | 1.06 | 4.83E−02 | 5.78 | 8100 | 8040 | 1.19E+00 | 1.12E+00 |
| BN034 | 1.09 | 4.17E−03 | 3.03 | 1085742 | 1153065 | 1.15E+00 | 1.06E+00 |
| BN035 | 1.28 | 2.43E−03 | 8.69 | 1536 | 1382 | 1.34E+00 | 1.05E+00 |
| BP001 | 0.66 | 2.38E−02 | 6.6 | 1940187 | 1425498 | 4.60E−01 | 7.10E−01 |
| BP006 | 1.1 | 1.75E−02 | 6.31 | 159425 | 155017 | 9.30E−01 | 8.50E−01 |
| BP010 | 1.14 | 6.88E−03 | 2.67 | 26340 | 26927 | 1.04E+00 | 9.10E−01 |
| BN012 | 0.89 | 1.94E−01 | 2.88 | 23140 | 23377 | 2.24E+00 | 2.53E+00 |
| BN019 | 1.05 | 4.30E−01 | 8.02 | 7997 | 8896 | 8.00E−01 | 7.70E−01 |
| BN020 | 1.2 | 1.55E−01 | 3.17 | 48644 | 44439 | 7.50E−01 | 6.20E−01 |
| BN021 | 0.97 | 8.63E−01 | 4.25 | 1307 | 1501 | 8.30E−01 | 8.60E−01 |
| BN022 | 0.99 | 9.63E−01 | 6.82 | 11218 | 11830 | 1.00E+00 | 1.01E+00 |
| BN023 | 0.97 | 6.78E−01 | 3.86 | 126845 | 132688 | 1.00E+00 | 1.03E+00 |
| BN025 | 1.05 | 1.40E−01 | 3.44 | 20487 | 21045 | 9.00E−01 | 8.60E−01 |
| BN027 | 1.05 | 1.33E−01 | 2.84 | 1344509 | 1395112 | 1.16E+00 | 1.11E+00 |
| BN028 | 1.18 | 1.83E−02 | 5.4 | 17235 | 15140 | 1.35E+00 | 1.13E+00 |
| BN032 | 1.84 | 3.83E−02 | 7.96 | 43590 | 24784 | 1.72E+00 | 1.18E+00 |
| BP002 | 1.1 | 6.15E−01 | 0.86 | 1620540 | 1386149 | 1.59E+00 | 1.45E+00 |
| BP003 | 1.13 | 5.39E−02 | 7.65 | 63222 | 65755 | 5.80E−01 | 5.10E−01 |
| BP007 | 1.07 | 1.04E−01 | 7.94 | 10773 | 10297 | 9.20E−01 | 8.60E−01 |
| BP008 | 0.93 | 9.97E−02 | 0.83 | 829808 | 802339 | 9.50E−01 | 1.02E+00 |
| BP009 | 1.1 | 2.13E−01 | 9.3 | 327296 | 342107 | 5.30E−01 | 4.90E−01 |
| BP011 | 1.03 | 5.92E−01 | 1.27 | 8840075 | 8346547 | 7.50E−01 | 7.30E−01 |
| BP012 | 1.01 | 8.52E−01 | 1.8 | 1033759 | 943633 | 7.60E−01 | 7.50E−01 |
| BP013 | 1.32 | 5.23E−04 | 4.2 | 18108 | 14608 | 6.55E−01 | 5.28E−01 |
| C002 | 0.81 | 2.63E−01 | 1.54 | 297532 | 358977 | 5.49E−01 | 6.75E−01 |
| C004 | 1.77 | 7.43E−02 | 1.85 | 34355 | 21962 | 2.05E+00 | 1.35E+00 |
| C008 | 1.01 | 7.58E−01 | 3.16 | 265519 | 269454 | 7.97E−01 | 8.26E−01 |
| C037 | 0.43 | 5.74E−05 | 2.38 | 50993 | 112252 | 8.36E−01 | 1.52E+00 |
| C118 | 0.67 | 1.27E−01 | 9.39 | 2822 | 4147 | 4.03E−01 | 5.76E−01 |
| C119 | 1.02 | 6.01E−01 | 5.09 | 52756 | 52133 | 9.29E−01 | 9.28E−01 |
| C135 | 0.38 | 2.35E−06 | 7.92 | 865535 | 1921900 | 1.41E+00 | 3.18E+00 |
| C138 | 0.68 | 1.04E−01 | 3.44 | 4050 | 7475 | 9.97E−01 | 1.87E+00 |
| C145 | 0.89 | 1.10E−02 | 7.92 | 400304 | 446188 | 1.15E+00 | 1.30E+00 |
| C150 | 0.76 | 3.62E−06 | 11.31 | 258215 | 307552 | 9.18E−01 | 1.18E+00 |
| DS01 | 1.37 | 2.08E−01 | 3.93 | 1555391 | 1171717 | 8.30E−01 | 6.00E−01 |
| DS02 | 1.14 | 3.26E−01 | 2.18 | 3533503 | 3054583 | 8.50E−01 | 7.40E−01 |
| DS03 | 0.99 | 9.08E−01 | 3.24 | 3170747 | 3224589 | 9.50E−01 | 9.70E−01 |
| DS04 | 0.96 | 9.22E−01 | 5.08 | 122022 | 84871 | 2.25E+00 | 2.35E+00 |
| DS05 | 1.14 | 3.14E−01 | 0.83 | 1130063 | 798806 | 1.42E+00 | 1.00E+00 |
| DS06 | 1.08 | 5.71E−01 | 1.34 | 218786 | 180806 | 1.40E+00 | 1.29E+00 |
| DS07 | 1.2 | 2.28E−01 | 1.94 | 17368 | 16639 | 2.13E+00 | 1.78E+00 |
| DS08 | 0.88 | 4.11E−01 | 3.12 | 186189 | 123792 | 1.09E+00 | 1.24E+00 |
| DS09 | 0.29 | 2.51E−01 | 2.62 | 47613 | 29940 | 2.53E+00 | 1.57E+00 |
| DS10 | 0.81 | 5.26E−01 | 3.97 | 933713 | 423378 | 8.10E−01 | 1.01E+00 |
| DS11 | 0.82 | 8.44E−03 | 2.19 | 1425036 | 1736561 | 9.56E−01 | 1.12E+00 |
| X006 | 1.58 | 1.13E−05 | 4.72 | 584777 | 486199 | 1.57E+00 | 1.23E+00 |
| X042 | 1.08 | 9.24E−03 | 0.93 | 6177600 | 5870581 | 1.17E+00 | 1.09E+00 |
| X043 | 2.13 | 5.63E−08 | 3.23 | 77323 | 36713 | 1.24E+00 | 5.89E−01 |
| X066 | 0.96 | 1.24E−01 | 1.79 | 2981686 | 3172801 | 1.08E+00 | 1.14E+00 |
| X082 | 58.1 | 1.34E−04 | 5.96 | 85956 | 1932 | 2.72E+01 | 7.65E−01 |
| X166 | 0.81 | 5.34E−14 | 2.06 | 95240 | 123926 | 8.44E−01 | 1.08E+00 |
| X188 | 0.91 | 1.37E−01 | 0.95 | 6828795 | 7306566 | 9.33E−01 | 1.01E+00 |
| X278 | 0.7 | 3.29E−17 | 4.68 | 67730 | 99873 | 7.41E−01 | 1.09E+00 |
| X280 | 0.7 | 1.15E−07 | 2.09 | 5139 | 7028 | 6.32E−01 | 8.97E−01 |
| X286 | 0.79 | 7.26E−10 | 13.98 | 970477 | 1272776 | 8.80E−01 | 1.15E+00 |
| X289 | 0.92 | 2.49E−02 | 1.74 | 138987 | 147397 | 1.07E+00 | 1.13E+00 |
| X293 | 0.87 | 6.94E−05 | 1.68 | 71255 | 81266 | 9.04E−01 | 1.03E+00 |
| X401 | 0.9 | 6.95E−02 | 4.97 | 7002 | 7817 | 8.57E−01 | 9.58E−01 |
| X407 | 0.48 | 2.35E−04 | 6.3 | 7271 | 15708 | 1.10E+00 | 2.37E+00 |
| X415 | 1.68 | 3.41E−08 | 3.51 | 120032 | 64672 | 1.53E+00 | 8.28E−01 |
| X459 | 0.98 | 6.95E−01 | 6.39 | 810 | 819 | 6.64E−01 | 6.77E−01 |
| X473 | 0.66 | 1.17E−17 | 11.51 | 71034 | 110084 | 6.24E−01 | 9.26E−01 |
| X508 | 0.62 | 2.44E−03 | 3.38 | 520672 | 1225244 | 1.88E+00 | 4.04E+00 |
| X511 | 0.92 | 1.63E−02 | 6.58 | 6919 | 7543 | 9.31E−01 | 1.02E+00 |
| X513 | 1.18 | 1.77E−05 | 8.54 | 127424 | 110203 | 1.10E+00 | 9.67E−01 |
| X531 | 1.07 | 1.16E−02 | 7.83 | 18846 | 17614 | 9.14E−01 | 8.34E−01 |
| X544 | 1.12 | 9.65E−02 | 3.53 | 4127 | 3493 | 9.19E−01 | 8.06E−01 |

### Example 2 Performances of Prediction Models for CRC and Precancerous Lesions

### I. Performance of the Prediction Model in the Training Set

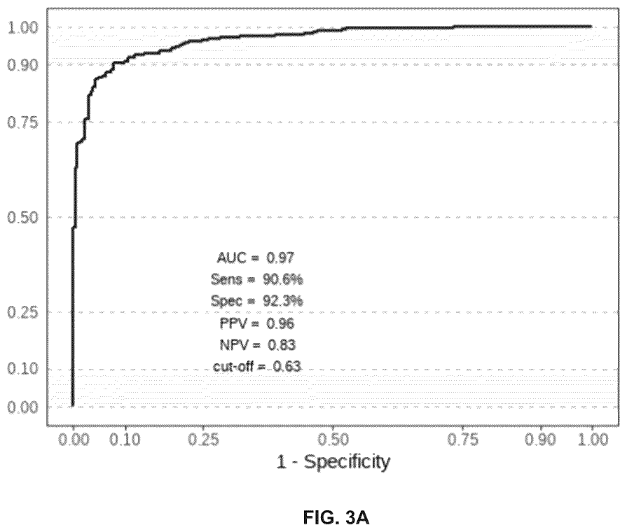

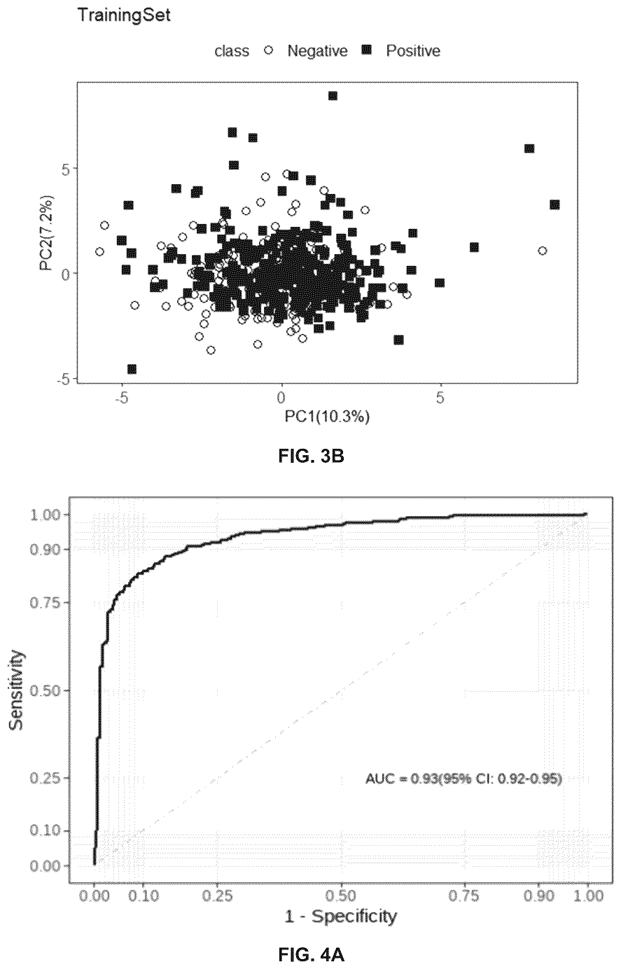

Based on the 81 metabolites selected above, prediction models were constructed based on logistic regression in the training cohort. FIG. 3A shows the performance of the prediction model for discriminating negative and positive group individuals in the training cohort. FIG. 3B is a Principal Component Analysis (PCA) plot of the prediction model for discriminating individuals of the positive group and the negative group in the training cohort. The model is trained based on the 81 metabolites listed in Table 1 and Table 2. As is shown in FIGS. 3A-3B, the AUC of this model to discriminate individuals with advanced neoplasia (advanced adenoma and CRC) from normal individuals and individuals with low-risk colorectal/polyps could achieve 0.97 (sensitivity=90.6%, specificity=92.3%, positive prediction value=0.96) in the training set at the selected threshold resulting in the highest accuracy (cut-off value=0.63), with PPV=0.96, and NPV=0.83 respectively.

### II. Performance for the Prediction Models in the Testing Cohort

Based on the prediction model constructed in the training cohort and the cut-off value (0.63), the performances of this model were further evaluated in the testing cohort. The performance of the prediction model for the positive group vs. negative group, as well as stage-specific performances, are shown in table 15 below:

TABLE 15
Summary of the performance of the prediction model for the positive
group vs. negative group, as well as stage-specific performances

| Group | AUC | Sensitivity | Specificity | Group | AUC | Sensitivity | Specificity |
|---|---|---|---|---|---|---|---|
| N + IP + CA vs | 0.86 | 0.723 | 0.842 | N vs | 0.94 | 0.887 | 0.870 |
| AA | | | | AA + EC + AC | | | |
| N + IP + CA vs | 0.90 | 0.799 | 0.842 | N vs EC | 0.94 | 0.872 | 0.870 |
| AA + EC | | | | | | | |
| N + IP + CA vs | 0.95 | 0.922 | 0.842 | N vs AC | 0.96 | 0.936 | 0.870 |
| EC + AC | | | | | | | |
| N + IP + CA vs | 0.93 | 0.887 | 0.842 | N vs AA | 0.87 | 0.723 | 0.870 |
| AA + EC + AC | | | | | | | |
| N + IP + CA vs | 0.93 | 0.872 | 0.842 | N vs AA + EC | 0.90 | 0.799 | 0.870 |
| EC | | | | | | | |
| N + IP + CA vs | 0.96 | 0.936 | 0.842 | N vs EC + AC | 0.95 | 0.922 | 0.870 |
| AC | | | | | | | |

In the above table, Normal is represented by N; Colorectal polyp is represented by IP; Low-risk adenoma is represented by CA; Advanced adenoma is represented by AA; Early-stage CRC is represented by EC; advanced CRC is represented by AC.

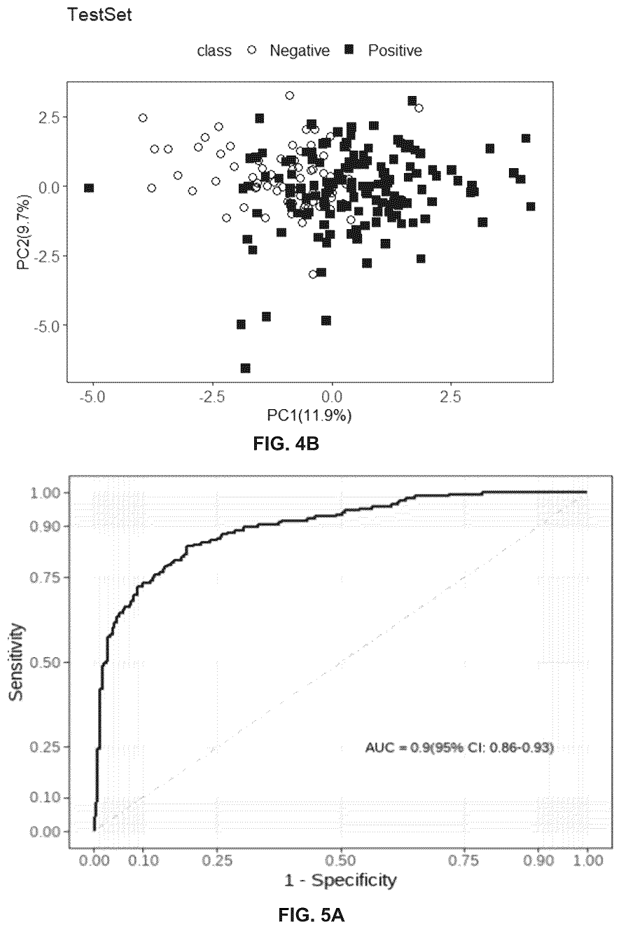

FIG. 4A shows the performance of the prediction model for discriminating negative and positive group individuals in the testing cohort. FIG. 4B is a PCA plot of the prediction model for discriminating negative and positive group individuals in the testing cohort. Specifically, as shown in FIG. 4A, the AUC of this model to discriminate advanced neoplasia (advanced adenoma and CRC) was 0.93 in the testing cohort (sensitivity=88.7%, specificity=84.2% at the cut-off value 0.63).

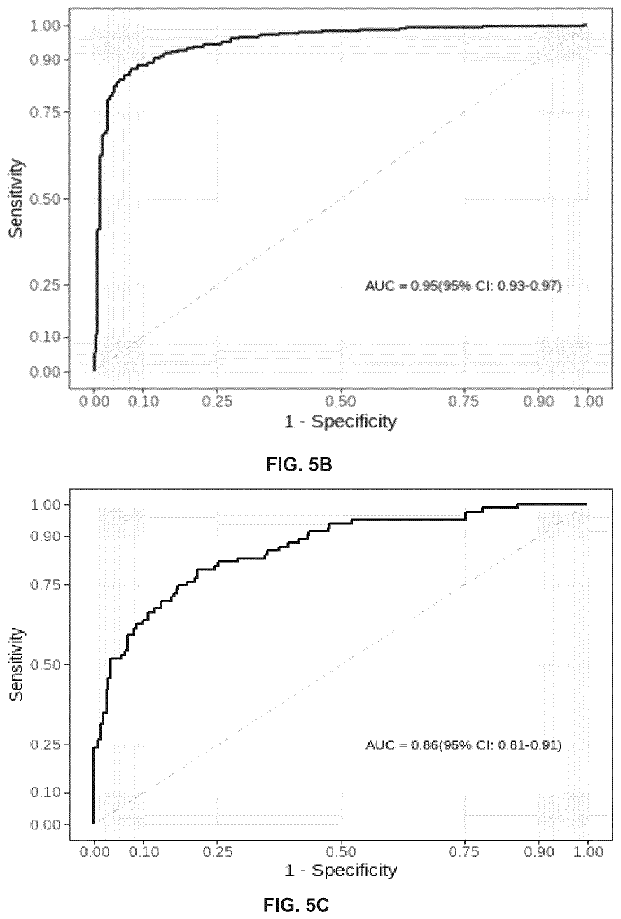

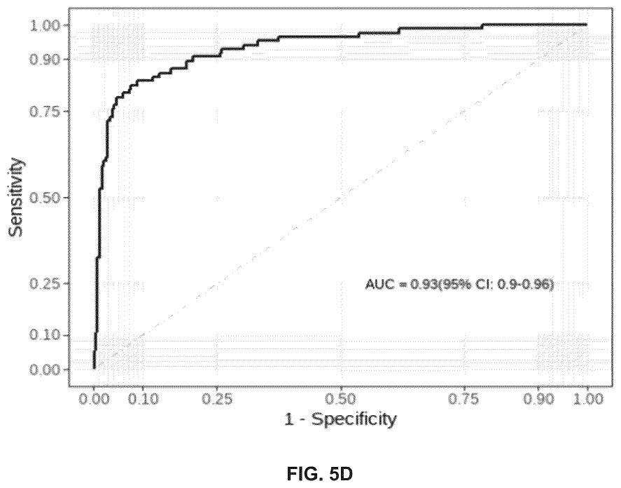

The performance of this model was also evaluated for different stages of CRC and CRA, and it was found that this model exhibited promising efficiency in discriminating early-stage CRC (stage 0 to I) and precancerous lesions (advanced adenoma). FIG. 5A shows the performance of the prediction model for discriminating negative subjects (including normal subjects, subjects having polyps, and subjects having low-risk colorectal adenomas) and positive subjects (including subjects having ACRA and subjects having early-stage CRC) in the testing cohort. FIG. 5B shows the performance of the prediction model for discriminating negative subjects (including normal subjects, subjects having polyps, and subjects having low-risk colorectal adenomas) and positive subjects (including subjects having early-stage CRC and subjects having middle/late-stage CRC) in the testing cohort. FIG. 5C shows the performance of the prediction model for discriminating negative subjects (including normal subjects, subjects having polyps, and subjects having low-risk colorectal adenomas) and positive subjects (including subjects having ACRA) in the testing cohort. FIG. 5D shows the performance of the prediction model for discriminating negative subjects (including normal subjects, subjects having polyps, and subjects having low-risk colorectal adenomas) and positive subjects (including subjects having early-stage CRC) in the testing cohort.

Specifically, the AUC is 0.86 for advanced adenoma (sensitivity=72.3%, specificity=84.2%) and 0.93 for stage 0 to I CRC (sensitivity=87.2%, specificity=84.2%) in the test set, which is significantly higher than fecal based FIT-DNA test and blood-based Septin9 test.

Collectively, the serum metabolites-based model provides a more accurate approach for early detection and diagnosis of CRC and precancerous lesions, which would greatly favor the treatment and decrease mortality caused by this disease.

III. The Performance of Prediction Models with a Variety of Combinations of the 81 Selected Metabolites

Prediction models based on individual metabolites, or a combination of any two or three within the panel of the 81 metabolites were also established. Performances of these models were listed in the table below and have been demonstrated in the following sections:

TABLE 16
Performances of prediction models based on individual metabolites
among the selected 81 metabolites (auc > 0.55).

| Meta ID | AUC | Sensitivity | Specificity | Cut-off | Meta ID | AUC | Sensitivity | Specificity | Cut-off |
|---|---|---|---|---|---|---|---|---|---|
| BN001 | 0.58 | 0.347 | 0.752 | 0.6533 | C135 | 0.67 | 0.507 | 0.752 | 0.6992 |
| BN002 | 0.6 | 0.411 | 0.752 | 0.6579 | C145 | 0.6 | 0.448 | 0.752 | 0.6864 |
| BN003 | 0.58 | 0.325 | 0.752 | 0.6571 | C150 | 0.6 | 0.34 | 0.752 | 0.71 |
| BN005 | 0.63 | 0.422 | 0.752 | 0.6527 | DS09 | 0.56 | 0.349 | 0.752 | 0.6675 |
| BN006 | 0.59 | 0.364 | 0.752 | 0.6706 | DS11 | 0.58 | 0.368 | 0.752 | 0.6912 |
| BN015 | 0.67 | 0.533 | 0.752 | 0.717 | X006 | 0.64 | 0.445 | 0.752 | 0.6736 |
| BN016 | 0.61 | 0.407 | 0.752 | 0.6729 | X043 | 0.72 | 0.587 | 0.752 | 0.6395 |
| BN017 | 0.58 | 0.377 | 0.752 | 0.6965 | X082 | 0.68 | 0.4 | 0.944 | 0.5914 |
| BN018 | 0.58 | 0.34 | 0.752 | 0.6832 | X166 | 0.67 | 0.478 | 0.752 | 0.7255 |
| BN029 | 0.56 | 0.308 | 0.752 | 0.6888 | X188 | 0.56 | 0.362 | 0.752 | 0.6804 |
| BN030 | 0.58 | 0.296 | 0.752 | 0.6942 | X278 | 0.7 | 0.565 | 0.752 | 0.7177 |
| BN035 | 0.56 | 0.37 | 0.752 | 0.6689 | X280 | 0.63 | 0.458 | 0.752 | 0.7113 |
| BP001 | 0.64 | 0.441 | 0.752 | 0.6778 | X286 | 0.65 | 0.48 | 0.752 | 0.7151 |
| BP010 | 0.57 | 0.39 | 0.752 | 0.6722 | X289 | 0.59 | 0.418 | 0.752 | 0.6846 |
| BN012 | 0.57 | 0.36 | 0.752 | 0.679 | X293 | 0.61 | 0.413 | 0.752 | 0.6966 |
| BN032 | 0.63 | 0.454 | 0.752 | 0.6546 | X407 | 0.66 | 0.484 | 0.752 | 0.6895 |
| BP002 | 0.58 | 0.338 | 0.752 | 0.6623 | X415 | 0.63 | 0.422 | 0.752 | 0.6865 |
| BP003 | 0.59 | 0.338 | 0.752 | 0.6699 | X473 | 0.71 | 0.574 | 0.752 | 0.7209 |
| BP009 | 0.56 | 0.353 | 0.752 | 0.6687 | X508 | 0.8 | 0.752 | 0.752 | 0.6798 |
| BP013 | 0.57 | 0.373 | 0.752 | 0.6881 | X513 | 0.61 | 0.37 | 0.752 | 0.6932 |
| C002 | 0.56 | 0.306 | 0.752 | 0.6736 | X531 | 0.56 | 0.317 | 0.752 | 0.6889 |
| C037 | 0.59 | 0.334 | 0.752 | 0.6903 | \ | \ | \ | \ | \ |

TABLE 17
Performances of prediction models based on the combination of
two metabolites among the selected 81 metabolites (auc > 0.75).

| The combination of | | | | |
|---|---|---|---|---|
| Meta ID1 and Meta ID2 | AUC | Sensitivity | Specificity | Cut-off |
| BN011 + X508 | 0.795 | 0.737 | 0.752 | 0.68 |
| BN032 + X508 | 0.77 | 0.764 | 0.752 | 0.6648 |
| C004 + X508 | 0.772 | 0.769 | 0.752 | 0.6703 |
| C008 + X508 | 0.8 | 0.749 | 0.752 | 0.6795 |
| C135 + X082 | 0.769 | 0.664 | 0.752 | 0.6199 |
| C135 + X508 | 0.752 | 0.63 | 0.752 | 0.7042 |
| DS09 + X508 | 0.802 | 0.747 | 0.752 | 0.6801 |
| X043 + X082 | 0.8 | 0.7 | 0.752 | 0.5682 |
| X043 + X166 | 0.754 | 0.634 | 0.752 | 0.6741 |
| X043 + X278 | 0.774 | 0.704 | 0.752 | 0.6542 |
| X043 + X280 | 0.753 | 0.619 | 0.752 | 0.6482 |
| X043 + X473 | 0.78 | 0.677 | 0.752 | 0.6614 |
| X082 + X166 | 0.761 | 0.64 | 0.752 | 0.66 |
| X082 + X278 | 0.778 | 0.675 | 0.752 | 0.66 |
| X082 + X407 | 0.763 | 0.66 | 0.752 | 0.6091 |
| X082 + X473 | 0.79 | 0.692 | 0.752 | 0.6585 |
| X082 + X508 | 0.846 | 0.839 | 0.752 | 0.6088 |
| X278 + X473 | 0.761 | 0.651 | 0.752 | 0.7072 |
| X407 + X508 | 0.751 | 0.655 | 0.752 | 0.6933 |

TABLE 18
Performances of prediction models based on the combination of
3 metabolites among the selected 81 metabolites (auc > 0.75).

| The combination of Meta ID1 | | | | |
|---|---|---|---|---|
| and Meta ID2 and Meta ID3 | AUC | Sensitivity | Specificity | Cut-off |
| BN006 + BN032 + X082 | 0.757 | 0.638 | 0.752 | 0.587 |
| BN006 + C135 + X082 | 0.757 | 0.625 | 0.752 | 0.6078 |
| BN006 + X006 + X082 | 0.765 | 0.651 | 0.752 | 0.6013 |
| BN006 + X043 + X082 | 0.807 | 0.677 | 0.752 | 0.5923 |
| BN006 + X043 + X166 | 0.756 | 0.595 | 0.752 | 0.6905 |
| BN006 + X043 + X278 | 0.772 | 0.702 | 0.752 | 0.66 |
| BN006 + X043 + X280 | 0.758 | 0.617 | 0.752 | 0.6607 |
| BN006 + X043 + X415 | 0.752 | 0.61 | 0.752 | 0.6624 |
| BN006 + X043 + X473 | 0.785 | 0.715 | 0.752 | 0.6519 |
| BN006 + X043 + X508 | 0.75 | 0.576 | 0.752 | 0.6631 |
| BN006 + X082 + X166 | 0.766 | 0.657 | 0.752 | 0.647 |
| BN006 + X082 + X278 | 0.782 | 0.672 | 0.752 | 0.6549 |
| BN006 + X082 + X280 | 0.755 | 0.653 | 0.752 | 0.622 |
| BN006 + X082 + X286 | 0.753 | 0.636 | 0.752 | 0.6406 |
| BN006 + X082 + X473 | 0.797 | 0.696 | 0.752 | 0.656 |
| BN006 + X082 + X508 | 0.8 | 0.677 | 0.752 | 0.6061 |
| BN006 + X278 + X473 | 0.764 | 0.666 | 0.752 | 0.709 |
| BN010 + X006 + X082 | 0.75 | 0.604 | 0.752 | 0.6032 |
| BN010 + X043 + X082 | 0.801 | 0.696 | 0.752 | 0.5692 |
| BN010 + X043 + X166 | 0.754 | 0.63 | 0.752 | 0.6746 |
| BN010 + X043 + X278 | 0.774 | 0.704 | 0.752 | 0.6531 |
| BN010 + X043 + X280 | 0.754 | 0.621 | 0.752 | 0.649 |
| BN010 + X043 + X473 | 0.779 | 0.683 | 0.752 | 0.6588 |
| BN010 + X082 + X166 | 0.761 | 0.645 | 0.752 | 0.6571 |
| BN010 + X082 + X278 | 0.779 | 0.677 | 0.752 | 0.6516 |
| BN010 + X082 + X473 | 0.791 | 0.681 | 0.752 | 0.6585 |
| BN010 + X082 + X508 | 0.801 | 0.801 | 0.752 | 0.6028 |
| BN010 + X278 + X473 | 0.761 | 0.664 | 0.752 | 0.7023 |
| BN011 + BN032 + X508 | 0.769 | 0.758 | 0.752 | 0.6645 |
| BN011 + C004 + X508 | 0.772 | 0.771 | 0.752 | 0.6694 |
| BN011 + C008 + X508 | 0.793 | 0.743 | 0.752 | 0.6793 |
| BN011 + C135 + X082 | 0.762 | 0.634 | 0.752 | 0.6199 |
| BN011 + C135 + X508 | 0.752 | 0.632 | 0.752 | 0.7039 |
| BN011 + DS09 + X508 | 0.797 | 0.739 | 0.752 | 0.6809 |
| BN011 + X043 + X082 | 0.8 | 0.698 | 0.752 | 0.5712 |
| BN011 + X043 + X166 | 0.755 | 0.627 | 0.752 | 0.6784 |
| BN011 + X043 + X278 | 0.776 | 0.726 | 0.752 | 0.6455 |
| BN011 + X043 + X280 | 0.753 | 0.604 | 0.752 | 0.6514 |
| BN011 + X043 + X415 | 0.751 | 0.617 | 0.752 | 0.6522 |
| BN011 + X043 + X473 | 0.782 | 0.681 | 0.752 | 0.6574 |
| BN011 + X082 + X166 | 0.762 | 0.63 | 0.752 | 0.6619 |
| BN011 + X082 + X278 | 0.779 | 0.675 | 0.752 | 0.6622 |
| BN011 + X082 + X407 | 0.764 | 0.664 | 0.752 | 0.6088 |
| BN011 + X082 + X473 | 0.79 | 0.692 | 0.752 | 0.6581 |
| BN011 + X082 + X508 | 0.827 | 0.837 | 0.752 | 0.6095 |
| BN011 + X278 + X473 | 0.761 | 0.649 | 0.752 | 0.7072 |
| BN011 + X407 + X508 | 0.75 | 0.655 | 0.752 | 0.6933 |
| BN028 + X006 + X278 | 0.754 | 0.615 | 0.752 | 0.694 |
| BN028 + X043 + X082 | 0.798 | 0.715 | 0.752 | 0.5707 |
| BN028 + X043 + X166 | 0.759 | 0.634 | 0.752 | 0.6778 |
| BN028 + X043 + X278 | 0.782 | 0.711 | 0.752 | 0.6518 |
| BN028 + X043 + X280 | 0.755 | 0.638 | 0.752 | 0.6504 |
| BN028 + X043 + X415 | 0.755 | 0.657 | 0.752 | 0.6475 |
| BN028 + X043 + X473 | 0.784 | 0.7 | 0.752 | 0.654 |
| BN028 + X082 + X166 | 0.764 | 0.634 | 0.752 | 0.6559 |
| BN028 + X082 + X278 | 0.786 | 0.657 | 0.752 | 0.6613 |
| BN028 + X082 + X286 | 0.754 | 0.647 | 0.752 | 0.6396 |
| BN028 + X082 + X473 | 0.79 | 0.666 | 0.752 | 0.6597 |
| BN028 + X082 + X508 | 0.808 | 0.807 | 0.752 | 0.5999 |
| BN028 + X166 + X473 | 0.75 | 0.612 | 0.752 | 0.7132 |
| BN028 + X278 + X415 | 0.754 | 0.675 | 0.752 | 0.6859 |
| BN028 + X278 + X473 | 0.771 | 0.67 | 0.752 | 0.6979 |
| BN028 + X286 + X473 | 0.759 | 0.662 | 0.752 | 0.6929 |
| BN032 + C004 + X508 | 0.766 | 0.734 | 0.752 | 0.6567 |
| BN032 + C008 + X508 | 0.77 | 0.764 | 0.752 | 0.6647 |
| BN032 + C037 + X082 | 0.765 | 0.642 | 0.752 | 0.5943 |
| BN032 + C135 + X082 | 0.784 | 0.662 | 0.752 | 0.6016 |
| BN032 + C135 + X473 | 0.75 | 0.63 | 0.752 | 0.7118 |
| BN032 + C135 + X508 | 0.753 | 0.668 | 0.752 | 0.6919 |
| BN032 + DS09 + X508 | 0.769 | 0.764 | 0.752 | 0.6657 |
| BN032 + DS11 + X082 | 0.754 | 0.672 | 0.752 | 0.598 |
| BN032 + X043 + X082 | 0.8 | 0.713 | 0.752 | 0.565 |
| BN032 + X043 + X166 | 0.755 | 0.636 | 0.752 | 0.6756 |
| BN032 + X043 + X278 | 0.775 | 0.7 | 0.752 | 0.6563 |
| BN032 + X043 + X280 | 0.754 | 0.617 | 0.752 | 0.6502 |
| BN032 + X043 + X415 | 0.75 | 0.606 | 0.752 | 0.6631 |
| BN032 + X043 + X473 | 0.782 | 0.67 | 0.752 | 0.6632 |
| BN032 + X082 + X166 | 0.772 | 0.657 | 0.752 | 0.6455 |
| BN032 + X082 + X278 | 0.788 | 0.685 | 0.752 | 0.6476 |
| BN032 + X082 + X280 | 0.764 | 0.653 | 0.752 | 0.6286 |
| BN032 + X082 + X286 | 0.761 | 0.664 | 0.752 | 0.6267 |
| BN032 + X082 + X293 | 0.755 | 0.625 | 0.752 | 0.6128 |
| BN032 + X082 + X407 | 0.773 | 0.636 | 0.752 | 0.5909 |
| BN032 + X082 + X415 | 0.771 | 0.649 | 0.752 | 0.6054 |
| BN032 + X082 + X473 | 0.801 | 0.7 | 0.752 | 0.6464 |
| BN032 + X082 + X508 | 0.839 | 0.846 | 0.752 | 0.5924 |
| BN032 + X082 + X513 | 0.759 | 0.638 | 0.752 | 0.6026 |
| BN032 + X166 + X473 | 0.75 | 0.604 | 0.752 | 0.7106 |
| BN032 + X278 + X473 | 0.765 | 0.666 | 0.752 | 0.6971 |
| BN032 + X407 + X508 | 0.758 | 0.692 | 0.752 | 0.6793 |
| BN032 + X415 + X473 | 0.757 | 0.653 | 0.752 | 0.6892 |
| BP013 + C135 + X473 | 0.754 | 0.617 | 0.752 | 0.7132 |
| BP013 + X006 + X082 | 0.764 | 0.64 | 0.752 | 0.607 |
| BP013 + X006 + X278 | 0.758 | 0.612 | 0.752 | 0.696 |
| BP013 + X043 + X082 | 0.8 | 0.722 | 0.752 | 0.5801 |
| BP013 + X043 + X166 | 0.77 | 0.681 | 0.752 | 0.6613 |
| BP013 + X043 + X278 | 0.781 | 0.685 | 0.752 | 0.6524 |
| BP013 + X043 + X280 | 0.756 | 0.651 | 0.752 | 0.6523 |
| BP013 + X043 + X293 | 0.75 | 0.63 | 0.752 | 0.6506 |
| BP013 + X043 + X407 | 0.75 | 0.625 | 0.752 | 0.6483 |
| BP013 + X043 + X415 | 0.753 | 0.64 | 0.752 | 0.655 |
| BP013 + X043 + X473 | 0.79 | 0.704 | 0.752 | 0.6522 |
| BP013 + X082 + X166 | 0.781 | 0.668 | 0.752 | 0.6469 |
| BP013 + X082 + X278 | 0.792 | 0.702 | 0.752 | 0.6496 |
| BP013 + X082 + X286 | 0.75 | 0.615 | 0.752 | 0.6463 |
| BP013 + X082 + X473 | 0.801 | 0.724 | 0.752 | 0.6493 |
| BP013 + X082 + X508 | 0.781 | 0.728 | 0.752 | 0.6007 |
| BP013 + X166 + X278 | 0.756 | 0.638 | 0.752 | 0.7042 |
| BP013 + X166 + X473 | 0.767 | 0.649 | 0.752 | 0.7053 |
| BP013 + X278 + X473 | 0.781 | 0.709 | 0.752 | 0.6885 |
| BP013 + X286 + X473 | 0.758 | 0.647 | 0.752 | 0.7059 |
| BP013 + X415 + X473 | 0.758 | 0.632 | 0.752 | 0.6999 |
| C002 + C135 + X082 | 0.755 | 0.604 | 0.752 | 0.6232 |
| C002 + X043 + X082 | 0.8 | 0.7 | 0.752 | 0.5674 |
| C002 + X043 + X166 | 0.754 | 0.64 | 0.752 | 0.6722 |
| C002 + X043 + X278 | 0.773 | 0.707 | 0.752 | 0.6515 |
| C002 + X043 + X280 | 0.753 | 0.617 | 0.752 | 0.6493 |
| C002 + X043 + X473 | 0.78 | 0.677 | 0.752 | 0.6626 |
| C002 + X082 + X166 | 0.762 | 0.634 | 0.752 | 0.6568 |
| C002 + X082 + X278 | 0.78 | 0.672 | 0.752 | 0.6587 |
| C002 + X082 + X407 | 0.758 | 0.649 | 0.752 | 0.6124 |
| C002 + X082 + X473 | 0.79 | 0.69 | 0.752 | 0.6607 |
| C002 + X082 + X508 | 0.828 | 0.812 | 0.752 | 0.6123 |
| C002 + X278 + X473 | 0.761 | 0.651 | 0.752 | 0.7049 |
| C004 + C008 + X508 | 0.769 | 0.777 | 0.752 | 0.6694 |
| C004 + C135 + X043 | 0.751 | 0.604 | 0.752 | 0.6518 |
| C004 + C135 + X082 | 0.759 | 0.625 | 0.752 | 0.6089 |
| C004 + DS09 + X508 | 0.773 | 0.764 | 0.752 | 0.671 |
| C004 + X006 + X082 | 0.756 | 0.619 | 0.752 | 0.5975 |
| C004 + X043 + X082 | 0.803 | 0.694 | 0.752 | 0.5746 |
| C004 + X043 + X166 | 0.756 | 0.647 | 0.752 | 0.6669 |
| C004 + X043 + X278 | 0.777 | 0.709 | 0.752 | 0.6497 |
| C004 + X043 + X280 | 0.758 | 0.638 | 0.752 | 0.6508 |
| C004 + X043 + X407 | 0.75 | 0.645 | 0.752 | 0.6327 |
| C004 + X043 + X415 | 0.758 | 0.617 | 0.752 | 0.657 |
| C004 + X043 + X473 | 0.783 | 0.683 | 0.752 | 0.6589 |
| C004 + X043 + X508 | 0.752 | 0.63 | 0.752 | 0.6344 |
| C004 + X082 + X166 | 0.764 | 0.623 | 0.752 | 0.6597 |
| C004 + X082 + X278 | 0.782 | 0.683 | 0.752 | 0.6529 |
| C004 + X082 + X407 | 0.752 | 0.612 | 0.752 | 0.5991 |
| C004 + X082 + X415 | 0.754 | 0.602 | 0.752 | 0.6117 |
| C004 + X082 + X473 | 0.793 | 0.698 | 0.752 | 0.6495 |
| C004 + X082 + X508 | 0.835 | 0.85 | 0.752 | 0.6025 |
| C004 + X278 + X473 | 0.765 | 0.657 | 0.752 | 0.7024 |
| C004 + X415 + X473 | 0.752 | 0.634 | 0.752 | 0.694 |
| C008 + DS09 + X508 | 0.801 | 0.747 | 0.752 | 0.6802 |
| C008 + X006 + X278 | 0.75 | 0.627 | 0.752 | 0.6944 |
| C008 + X043 + X082 | 0.8 | 0.702 | 0.752 | 0.5684 |
| C008 + X043 + X166 | 0.76 | 0.651 | 0.752 | 0.6681 |
| C008 + X043 + X278 | 0.782 | 0.726 | 0.752 | 0.6454 |
| C008 + X043 + X280 | 0.756 | 0.63 | 0.752 | 0.6471 |
| C008 + X043 + X473 | 0.781 | 0.683 | 0.752 | 0.6605 |
| C008 + X082 + X166 | 0.77 | 0.653 | 0.752 | 0.6547 |
| C008 + X082 + X278 | 0.791 | 0.702 | 0.752 | 0.6416 |
| C008 + X082 + X286 | 0.75 | 0.64 | 0.752 | 0.637 |
| C008 + X082 + X473 | 0.788 | 0.681 | 0.752 | 0.6605 |
| C008 + X082 + X508 | 0.843 | 0.839 | 0.752 | 0.6076 |
| C008 + X278 + X473 | 0.766 | 0.675 | 0.752 | 0.6992 |
| C037 + C135 + X082 | 0.762 | 0.625 | 0.752 | 0.6283 |
| C037 + X006 + X082 | 0.763 | 0.649 | 0.752 | 0.6052 |
| C037 + X043 + X082 | 0.806 | 0.702 | 0.752 | 0.5781 |
| C037 + X043 + X166 | 0.764 | 0.668 | 0.752 | 0.6675 |
| C037 + X043 + X278 | 0.775 | 0.696 | 0.752 | 0.6585 |
| C037 + X043 + X280 | 0.762 | 0.619 | 0.752 | 0.6596 |
| C037 + X043 + X407 | 0.753 | 0.617 | 0.752 | 0.6502 |
| C037 + X043 + X415 | 0.761 | 0.615 | 0.752 | 0.667 |
| C037 + X043 + X473 | 0.787 | 0.677 | 0.752 | 0.6629 |
| C037 + X082 + X166 | 0.771 | 0.649 | 0.752 | 0.6624 |
| C037 + X082 + X278 | 0.778 | 0.672 | 0.752 | 0.6619 |
| C037 + X082 + X280 | 0.757 | 0.636 | 0.752 | 0.6528 |
| C037 + X082 + X286 | 0.757 | 0.653 | 0.752 | 0.6496 |
| C037 + X082 + X407 | 0.76 | 0.636 | 0.752 | 0.6283 |
| C037 + X082 + X415 | 0.755 | 0.623 | 0.752 | 0.6153 |
| C037 + X082 + X473 | 0.796 | 0.679 | 0.752 | 0.6653 |
| C037 + X082 + X508 | 0.815 | 0.792 | 0.752 | 0.6237 |
| C037 + X166 + X473 | 0.752 | 0.619 | 0.752 | 0.7176 |
| C037 + X278 + X473 | 0.763 | 0.677 | 0.752 | 0.7017 |
| C037 + X286 + X473 | 0.751 | 0.647 | 0.752 | 0.7004 |
| C037 + X415 + X473 | 0.754 | 0.664 | 0.752 | 0.69 |
| C118 + C135 + X082 | 0.753 | 0.61 | 0.752 | 0.6287 |
| C118 + X043 + X082 | 0.802 | 0.7 | 0.752 | 0.5708 |
| C118 + X043 + X166 | 0.754 | 0.627 | 0.752 | 0.677 |
| C118 + X043 + X278 | 0.776 | 0.707 | 0.752 | 0.6577 |
| C118 + X043 + X280 | 0.753 | 0.602 | 0.752 | 0.6542 |
| C118 + X043 + X415 | 0.75 | 0.6 | 0.752 | 0.664 |
| C118 + X043 + X473 | 0.781 | 0.683 | 0.752 | 0.6583 |
| C118 + X082 + X166 | 0.762 | 0.632 | 0.752 | 0.6634 |
| C118 + X082 + X278 | 0.779 | 0.679 | 0.752 | 0.6581 |
| C118 + X082 + X473 | 0.792 | 0.683 | 0.752 | 0.6604 |
| C118 + X082 + X508 | 0.826 | 0.818 | 0.752 | 0.6131 |
| C118 + X278 + X473 | 0.761 | 0.636 | 0.752 | 0.7138 |
| C119 + X006 + X082 | 0.751 | 0.612 | 0.752 | 0.6047 |
| C119 + X043 + X082 | 0.799 | 0.69 | 0.752 | 0.5797 |
| C119 + X043 + X166 | 0.757 | 0.649 | 0.752 | 0.6721 |
| C119 + X043 + X278 | 0.774 | 0.696 | 0.752 | 0.6567 |
| C119 + X043 + X280 | 0.754 | 0.63 | 0.752 | 0.6492 |
| C119 + X043 + X473 | 0.781 | 0.685 | 0.752 | 0.6615 |
| C119 + X082 + X166 | 0.763 | 0.642 | 0.752 | 0.6532 |
| C119 + X082 + X278 | 0.778 | 0.666 | 0.752 | 0.6631 |
| C119 + X082 + X473 | 0.791 | 0.672 | 0.752 | 0.6595 |
| C119 + X082 + X508 | 0.78 | 0.69 | 0.752 | 0.6118 |
| C119 + X278 + X473 | 0.762 | 0.651 | 0.752 | 0.706 |
| C119 + X286 + X473 | 0.75 | 0.63 | 0.752 | 0.7055 |
| C135 + C138 + X082 | 0.754 | 0.606 | 0.752 | 0.6228 |
| C135 + C150 + X043 | 0.753 | 0.606 | 0.752 | 0.6593 |
| C135 + C150 + X473 | 0.75 | 0.647 | 0.752 | 0.7083 |
| C135 + DS09 + X082 | 0.767 | 0.636 | 0.752 | 0.6209 |
| C135 + DS09 + X508 | 0.753 | 0.632 | 0.752 | 0.7048 |
| C135 + DS11 + X043 | 0.759 | 0.63 | 0.752 | 0.6569 |
| C135 + DS11 + X082 | 0.75 | 0.632 | 0.752 | 0.6349 |
| C135 + X006 + X043 | 0.756 | 0.612 | 0.752 | 0.6503 |
| C135 + X006 + X082 | 0.779 | 0.649 | 0.752 | 0.608 |
| C135 + X006 + X278 | 0.752 | 0.597 | 0.752 | 0.7096 |
| C135 + X006 + X473 | 0.759 | 0.67 | 0.752 | 0.6935 |
| C135 + X042 + X043 | 0.75 | 0.617 | 0.752 | 0.661 |
| C135 + X043 + X082 | 0.813 | 0.717 | 0.752 | 0.5795 |
| C135 + X043 + X166 | 0.768 | 0.683 | 0.752 | 0.665 |
| C135 + X043 + X278 | 0.784 | 0.709 | 0.752 | 0.6607 |
| C135 + X043 + X280 | 0.771 | 0.662 | 0.752 | 0.657 |
| C135 + X043 + X286 | 0.75 | 0.608 | 0.752 | 0.6762 |
| C135 + X043 + X293 | 0.757 | 0.595 | 0.752 | 0.6549 |
| C135 + X043 + X407 | 0.762 | 0.608 | 0.752 | 0.6615 |
| C135 + X043 + X415 | 0.767 | 0.617 | 0.752 | 0.6668 |
| C135 + X043 + X473 | 0.795 | 0.687 | 0.752 | 0.6626 |
| C135 + X043 + X508 | 0.758 | 0.595 | 0.752 | 0.6598 |
| C135 + X043 + X513 | 0.761 | 0.627 | 0.752 | 0.6541 |
| C135 + X082 + X166 | 0.771 | 0.642 | 0.752 | 0.662 |
| C135 + X082 + X188 | 0.751 | 0.606 | 0.752 | 0.6219 |
| C135 + X082 + X278 | 0.786 | 0.679 | 0.752 | 0.6623 |
| C135 + X082 + X280 | 0.773 | 0.666 | 0.752 | 0.6508 |
| C135 + X082 + X286 | 0.764 | 0.655 | 0.752 | 0.6486 |
| C135 + X082 + X293 | 0.759 | 0.621 | 0.752 | 0.6345 |
| C135 + X082 + X407 | 0.784 | 0.687 | 0.752 | 0.6324 |
| C135 + X082 + X415 | 0.766 | 0.625 | 0.752 | 0.6154 |
| C135 + X082 + X459 | 0.759 | 0.606 | 0.752 | 0.6197 |
| C135 + X082 + X473 | 0.807 | 0.696 | 0.752 | 0.6642 |
| C135 + X082 + X508 | 0.821 | 0.788 | 0.752 | 0.628 |
| C135 + X082 + X513 | 0.764 | 0.597 | 0.752 | 0.629 |
| C135 + X166 + X473 | 0.758 | 0.632 | 0.752 | 0.7149 |
| C135 + X278 + X473 | 0.773 | 0.685 | 0.752 | 0.6988 |
| C135 + X286 + X473 | 0.759 | 0.651 | 0.752 | 0.7051 |
| C135 + X407 + X473 | 0.75 | 0.642 | 0.752 | 0.7092 |
| C135 + X415 + X473 | 0.761 | 0.662 | 0.752 | 0.698 |
| C135 + X473 + X508 | 0.755 | 0.632 | 0.752 | 0.7098 |
| C138 + X043 + X082 | 0.8 | 0.713 | 0.752 | 0.564 |
| C138 + X043 + X166 | 0.754 | 0.63 | 0.752 | 0.6795 |
| C138 + X043 + X278 | 0.774 | 0.704 | 0.752 | 0.652 |
| C138 + X043 + X280 | 0.755 | 0.632 | 0.752 | 0.6485 |
| C138 + X043 + X415 | 0.75 | 0.6 | 0.752 | 0.6626 |
| C138 + X043 + X473 | 0.781 | 0.683 | 0.752 | 0.658 |
| C138 + X082 + X166 | 0.762 | 0.64 | 0.752 | 0.6585 |
| C138 + X082 + X278 | 0.778 | 0.679 | 0.752 | 0.6585 |
| C138 + X082 + X473 | 0.792 | 0.685 | 0.752 | 0.6596 |
| C138 + X082 + X508 | 0.814 | 0.775 | 0.752 | 0.6153 |
| C138 + X278 + X473 | 0.761 | 0.651 | 0.752 | 0.707 |
| C145 + X006 + X082 | 0.755 | 0.642 | 0.752 | 0.5978 |
| C145 + X043 + X082 | 0.798 | 0.7 | 0.752 | 0.5707 |
| C145 + X043 + X166 | 0.753 | 0.636 | 0.752 | 0.6758 |
| C145 + X043 + X278 | 0.775 | 0.717 | 0.752 | 0.6507 |
| C145 + X043 + X280 | 0.755 | 0.642 | 0.752 | 0.6499 |
| C145 + X043 + X415 | 0.753 | 0.61 | 0.752 | 0.6557 |
| C145 + X043 + X473 | 0.783 | 0.681 | 0.752 | 0.6597 |
| C145 + X043 + X513 | 0.756 | 0.642 | 0.752 | 0.6548 |
| C145 + X082 + X166 | 0.761 | 0.623 | 0.752 | 0.6611 |
| C145 + X082 + X278 | 0.778 | 0.683 | 0.752 | 0.6511 |
| C145 + X082 + X415 | 0.756 | 0.602 | 0.752 | 0.6131 |
| C145 + X082 + X473 | 0.795 | 0.704 | 0.752 | 0.6525 |
| C145 + X082 + X508 | 0.789 | 0.737 | 0.752 | 0.6197 |
| C145 + X082 + X513 | 0.771 | 0.685 | 0.752 | 0.6084 |
| C145 + X278 + X473 | 0.763 | 0.666 | 0.752 | 0.6985 |
| C145 + X415 + X473 | 0.756 | 0.653 | 0.752 | 0.6923 |
| C145 + X473 + X513 | 0.75 | 0.638 | 0.752 | 0.7076 |
| C150 + X006 + X082 | 0.765 | 0.657 | 0.752 | 0.6032 |
| C150 + X006 + X473 | 0.751 | 0.664 | 0.752 | 0.6877 |
| C150 + X043 + X082 | 0.803 | 0.709 | 0.752 | 0.5759 |
| C150 + X043 + X166 | 0.758 | 0.672 | 0.752 | 0.6694 |
| C150 + X043 + X278 | 0.776 | 0.694 | 0.752 | 0.6674 |
| C150 + X043 + X280 | 0.763 | 0.666 | 0.752 | 0.6547 |
| C150 + X043 + X293 | 0.752 | 0.617 | 0.752 | 0.6526 |
| C150 + X043 + X407 | 0.751 | 0.612 | 0.752 | 0.6554 |
| C150 + X043 + X415 | 0.764 | 0.638 | 0.752 | 0.6611 |
| C150 + X043 + X473 | 0.787 | 0.687 | 0.752 | 0.6632 |
| C150 + X043 + X508 | 0.751 | 0.623 | 0.752 | 0.6489 |
| C150 + X082 + X166 | 0.765 | 0.642 | 0.752 | 0.6636 |
| C150 + X082 + X278 | 0.783 | 0.677 | 0.752 | 0.6563 |
| C150 + X082 + X415 | 0.756 | 0.638 | 0.752 | 0.61 |
| C150 + X082 + X473 | 0.799 | 0.726 | 0.752 | 0.6509 |
| C150 + X082 + X508 | 0.78 | 0.715 | 0.752 | 0.6283 |
| C150 + X166 + X473 | 0.751 | 0.642 | 0.752 | 0.7099 |
| C150 + X278 + X473 | 0.769 | 0.653 | 0.752 | 0.7125 |
| C150 + X286 + X473 | 0.752 | 0.649 | 0.752 | 0.7095 |
| C150 + X415 + X473 | 0.759 | 0.651 | 0.752 | 0.6999 |
| DS05 + X006 + X082 | 0.75 | 0.602 | 0.752 | 0.6023 |
| DS05 + X043 + X082 | 0.798 | 0.698 | 0.752 | 0.5724 |
| DS05 + X043 + X166 | 0.754 | 0.636 | 0.752 | 0.6762 |
| DS05 + X043 + X278 | 0.774 | 0.7 | 0.752 | 0.6577 |
| DS05 + X043 + X280 | 0.755 | 0.627 | 0.752 | 0.6457 |
| DS05 + X043 + X473 | 0.78 | 0.677 | 0.752 | 0.6642 |
| DS05 + X082 + X166 | 0.764 | 0.634 | 0.752 | 0.66 |
| DS05 + X082 + X278 | 0.779 | 0.679 | 0.752 | 0.6579 |
| DS05 + X082 + X473 | 0.791 | 0.681 | 0.752 | 0.6572 |
| DS05 + X082 + X508 | 0.808 | 0.809 | 0.752 | 0.605 |
| DS05 + X278 + X473 | 0.761 | 0.66 | 0.752 | 0.7064 |
| DS09 + X043 + X082 | 0.801 | 0.711 | 0.752 | 0.5665 |
| DS09 + X043 + X166 | 0.757 | 0.636 | 0.752 | 0.6743 |
| DS09 + X043 + X278 | 0.777 | 0.707 | 0.752 | 0.6544 |
| DS09 + X043 + X280 | 0.757 | 0.612 | 0.752 | 0.6501 |
| DS09 + X043 + X415 | 0.751 | 0.608 | 0.752 | 0.6604 |
| DS09 + X043 + X473 | 0.781 | 0.677 | 0.752 | 0.6626 |
| DS09 + X082 + X166 | 0.762 | 0.638 | 0.752 | 0.662 |
| DS09 + X082 + X278 | 0.78 | 0.677 | 0.752 | 0.6617 |
| DS09 + X082 + X407 | 0.767 | 0.672 | 0.752 | 0.61 |
| DS09 + X082 + X473 | 0.79 | 0.694 | 0.752 | 0.6589 |
| DS09 + X082 + X508 | 0.848 | 0.839 | 0.752 | 0.6096 |
| DS09 + X278 + X473 | 0.762 | 0.651 | 0.752 | 0.7082 |
| DS09 + X407 + X508 | 0.751 | 0.655 | 0.752 | 0.694 |
| DS11 + X006 + X043 | 0.757 | 0.63 | 0.752 | 0.6479 |
| DS11 + X006 + X082 | 0.764 | 0.664 | 0.752 | 0.5879 |
| DS11 + X043 + X082 | 0.807 | 0.698 | 0.752 | 0.5787 |
| DS11 + X043 + X166 | 0.761 | 0.649 | 0.752 | 0.6782 |
| DS11 + X043 + X278 | 0.776 | 0.681 | 0.752 | 0.6688 |
| DS11 + X043 + X280 | 0.762 | 0.685 | 0.752 | 0.6464 |
| DS11 + X043 + X407 | 0.752 | 0.619 | 0.752 | 0.6499 |
| DS11 + X043 + X415 | 0.761 | 0.602 | 0.752 | 0.6603 |
| DS11 + X043 + X473 | 0.787 | 0.709 | 0.752 | 0.6486 |
| DS11 + X043 + X513 | 0.753 | 0.634 | 0.752 | 0.6494 |
| DS11 + X082 + X166 | 0.765 | 0.645 | 0.752 | 0.6578 |
| DS11 + X082 + X278 | 0.779 | 0.672 | 0.752 | 0.6534 |
| DS11 + X082 + X473 | 0.79 | 0.687 | 0.752 | 0.6581 |
| DS11 + X082 + X508 | 0.791 | 0.745 | 0.752 | 0.6204 |
| DS11 + X278 + X473 | 0.76 | 0.66 | 0.752 | 0.7086 |
| X006 + X042 + X082 | 0.758 | 0.63 | 0.752 | 0.6059 |
| X006 + X042 + X278 | 0.752 | 0.608 | 0.752 | 0.7014 |
| X006 + X043 + X082 | 0.81 | 0.717 | 0.752 | 0.5721 |
| X006 + X043 + X166 | 0.768 | 0.617 | 0.752 | 0.6741 |
| X006 + X043 + X278 | 0.784 | 0.724 | 0.752 | 0.6435 |
| X006 + X043 + X280 | 0.764 | 0.657 | 0.752 | 0.6432 |
| X006 + X043 + X293 | 0.756 | 0.625 | 0.752 | 0.6451 |
| X006 + X043 + X407 | 0.757 | 0.636 | 0.752 | 0.6349 |
| X006 + X043 + X415 | 0.763 | 0.645 | 0.752 | 0.6466 |
| X006 + X043 + X473 | 0.782 | 0.67 | 0.752 | 0.6568 |
| X006 + X043 + X508 | 0.755 | 0.675 | 0.752 | 0.6285 |
| X006 + X082 + X166 | 0.796 | 0.704 | 0.752 | 0.6231 |
| X006 + X082 + X278 | 0.8 | 0.704 | 0.752 | 0.6255 |
| X006 + X082 + X280 | 0.771 | 0.64 | 0.752 | 0.6156 |
| X006 + X082 + X286 | 0.778 | 0.67 | 0.752 | 0.6181 |
| X006 + X082 + X289 | 0.753 | 0.638 | 0.752 | 0.5966 |
| X006 + X082 + X293 | 0.774 | 0.679 | 0.752 | 0.6144 |
| X006 + X082 + X407 | 0.762 | 0.634 | 0.752 | 0.6046 |
| X006 + X082 + X415 | 0.791 | 0.704 | 0.752 | 0.5932 |
| X006 + X082 + X473 | 0.808 | 0.739 | 0.752 | 0.6326 |
| X006 + X082 + X508 | 0.808 | 0.685 | 0.752 | 0.5986 |
| X006 + X082 + X511 | 0.754 | 0.642 | 0.752 | 0.5969 |
| X006 + X082 + X513 | 0.774 | 0.672 | 0.752 | 0.6025 |
| X006 + X082 + X531 | 0.763 | 0.623 | 0.752 | 0.613 |
| X006 + X082 + X544 | 0.752 | 0.623 | 0.752 | 0.6062 |
| X006 + X166 + X278 | 0.766 | 0.649 | 0.752 | 0.6904 |
| X006 + X166 + X415 | 0.763 | 0.621 | 0.752 | 0.6815 |
| X006 + X166 + X473 | 0.766 | 0.668 | 0.752 | 0.6826 |
| X006 + X166 + X513 | 0.753 | 0.638 | 0.752 | 0.6836 |
| X006 + X278 + X280 | 0.754 | 0.64 | 0.752 | 0.6995 |
| X006 + X278 + X415 | 0.771 | 0.649 | 0.752 | 0.6801 |
| X006 + X278 + X473 | 0.777 | 0.681 | 0.752 | 0.6873 |
| X006 + X278 + X508 | 0.756 | 0.647 | 0.752 | 0.6957 |
| X006 + X278 + X513 | 0.759 | 0.666 | 0.752 | 0.6823 |
| X006 + X278 + X544 | 0.752 | 0.612 | 0.752 | 0.6973 |
| X006 + X286 + X473 | 0.757 | 0.67 | 0.752 | 0.6801 |
| X006 + X415 + X473 | 0.77 | 0.677 | 0.752 | 0.674 |
| X006 + X473 + X508 | 0.754 | 0.66 | 0.752 | 0.6874 |
| X042 + X043 + X082 | 0.805 | 0.715 | 0.752 | 0.5863 |
| X042 + X043 + X166 | 0.767 | 0.685 | 0.752 | 0.6585 |
| X042 + X043 + X278 | 0.78 | 0.713 | 0.752 | 0.6479 |
| X042 + X043 + X280 | 0.76 | 0.657 | 0.752 | 0.6565 |
| X042 + X043 + X286 | 0.77 | 0.636 | 0.752 | 0.6738 |
| X042 + X043 + X289 | 0.751 | 0.621 | 0.752 | 0.6541 |
| X042 + X043 + X293 | 0.752 | 0.619 | 0.752 | 0.6549 |
| X042 + X043 + X415 | 0.758 | 0.638 | 0.752 | 0.6585 |
| X042 + X043 + X473 | 0.788 | 0.715 | 0.752 | 0.6438 |
| X042 + X082 + X166 | 0.769 | 0.664 | 0.752 | 0.647 |
| X042 + X082 + X278 | 0.788 | 0.672 | 0.752 | 0.6554 |
| X042 + X082 + X286 | 0.779 | 0.655 | 0.752 | 0.6484 |
| X042 + X082 + X473 | 0.793 | 0.696 | 0.752 | 0.6523 |
| X042 + X082 + X508 | 0.776 | 0.69 | 0.752 | 0.61 |
| X042 + X166 + X473 | 0.753 | 0.645 | 0.752 | 0.7118 |
| X042 + X278 + X286 | 0.761 | 0.634 | 0.752 | 0.713 |
| X042 + X278 + X473 | 0.772 | 0.655 | 0.752 | 0.7089 |
| X042 + X286 + X415 | 0.75 | 0.632 | 0.752 | 0.6859 |
| X042 + X286 + X473 | 0.781 | 0.677 | 0.752 | 0.696 |
| X043 + X066 + X082 | 0.798 | 0.702 | 0.752 | 0.5716 |
| X043 + X066 + X166 | 0.752 | 0.638 | 0.752 | 0.6684 |
| X043 + X066 + X278 | 0.774 | 0.692 | 0.752 | 0.6594 |
| X043 + X066 + X280 | 0.751 | 0.63 | 0.752 | 0.6489 |
| X043 + X066 + X415 | 0.751 | 0.606 | 0.752 | 0.6521 |
| X043 + X066 + X473 | 0.781 | 0.683 | 0.752 | 0.6515 |
| X043 + X082 + X166 | 0.817 | 0.745 | 0.752 | 0.5964 |
| X043 + X082 + X188 | 0.8 | 0.702 | 0.752 | 0.5684 |
| X043 + X082 + X278 | 0.827 | 0.767 | 0.752 | 0.5827 |
| X043 + X082 + X280 | 0.821 | 0.739 | 0.752 | 0.5803 |
| X043 + X082 + X286 | 0.802 | 0.702 | 0.752 | 0.5849 |
| X043 + X082 + X289 | 0.801 | 0.715 | 0.752 | 0.5692 |
| X043 + X082 + X293 | 0.811 | 0.709 | 0.752 | 0.572 |
| X043 + X082 + X401 | 0.8 | 0.704 | 0.752 | 0.5685 |
| X043 + X082 + X407 | 0.808 | 0.726 | 0.752 | 0.5662 |
| X043 + X082 + X415 | 0.821 | 0.717 | 0.752 | 0.5922 |
| X043 + X082 + X459 | 0.799 | 0.702 | 0.752 | 0.5706 |
| X043 + X082 + X473 | 0.841 | 0.764 | 0.752 | 0.5884 |
| X043 + X082 + X508 | 0.825 | 0.743 | 0.752 | 0.5658 |
| X043 + X082 + X511 | 0.802 | 0.7 | 0.752 | 0.5722 |
| X043 + X082 + X513 | 0.812 | 0.713 | 0.752 | 0.5854 |
| X043 + X082 + X531 | 0.803 | 0.7 | 0.752 | 0.584 |
| X043 + X082 + X544 | 0.8 | 0.711 | 0.752 | 0.5702 |
| X043 + X166 + X188 | 0.755 | 0.625 | 0.752 | 0.6793 |
| X043 + X166 + X278 | 0.783 | 0.73 | 0.752 | 0.6619 |
| X043 + X166 + X280 | 0.778 | 0.694 | 0.752 | 0.6541 |
| X043 + X166 + X286 | 0.753 | 0.64 | 0.752 | 0.6778 |
| X043 + X166 + X289 | 0.755 | 0.662 | 0.752 | 0.6666 |
| X043 + X166 + X293 | 0.763 | 0.685 | 0.752 | 0.661 |
| X043 + X166 + X401 | 0.753 | 0.63 | 0.752 | 0.6734 |
| X043 + X166 + X407 | 0.773 | 0.704 | 0.752 | 0.6543 |
| X043 + X166 + X415 | 0.781 | 0.69 | 0.752 | 0.6663 |
| X043 + X166 + X459 | 0.755 | 0.64 | 0.752 | 0.6734 |
| X043 + X166 + X473 | 0.796 | 0.702 | 0.752 | 0.6652 |
| X043 + X166 + X508 | 0.763 | 0.664 | 0.752 | 0.6717 |
| X043 + X166 + X511 | 0.758 | 0.649 | 0.752 | 0.6744 |
| X043 + X166 + X513 | 0.774 | 0.687 | 0.752 | 0.6609 |
| X043 + X166 + X531 | 0.766 | 0.653 | 0.752 | 0.6691 |
| X043 + X166 + X544 | 0.763 | 0.664 | 0.752 | 0.6665 |
| X043 + X188 + X278 | 0.775 | 0.726 | 0.752 | 0.6456 |
| X043 + X188 + X280 | 0.753 | 0.621 | 0.752 | 0.6509 |
| X043 + X188 + X473 | 0.78 | 0.67 | 0.752 | 0.6602 |
| X043 + X278 + X280 | 0.788 | 0.696 | 0.752 | 0.6594 |
| X043 + X278 + X286 | 0.773 | 0.668 | 0.752 | 0.6683 |
| X043 + X278 + X289 | 0.775 | 0.707 | 0.752 | 0.6536 |
| X043 + X278 + X293 | 0.776 | 0.719 | 0.752 | 0.6496 |
| X043 + X278 + X401 | 0.775 | 0.702 | 0.752 | 0.6548 |
| X043 + X278 + X407 | 0.781 | 0.702 | 0.752 | 0.658 |
| X043 + X278 + X415 | 0.788 | 0.711 | 0.752 | 0.6436 |
| X043 + X278 + X459 | 0.773 | 0.704 | 0.752 | 0.6557 |
| X043 + X278 + X473 | 0.808 | 0.758 | 0.752 | 0.6357 |
| X043 + X278 + X508 | 0.783 | 0.726 | 0.752 | 0.6556 |
| X043 + X278 + X511 | 0.775 | 0.719 | 0.752 | 0.6496 |
| X043 + X278 + X513 | 0.78 | 0.687 | 0.752 | 0.6579 |
| X043 + X278 + X531 | 0.772 | 0.694 | 0.752 | 0.6569 |
| X043 + X278 + X544 | 0.781 | 0.709 | 0.752 | 0.6513 |
| X043 + X280 + X286 | 0.764 | 0.642 | 0.752 | 0.6678 |
| X043 + X280 + X289 | 0.754 | 0.67 | 0.752 | 0.6403 |
| X043 + X280 + X293 | 0.765 | 0.702 | 0.752 | 0.6357 |
| X043 + X280 + X401 | 0.753 | 0.651 | 0.752 | 0.6422 |
| X043 + X280 + X407 | 0.765 | 0.612 | 0.752 | 0.6614 |
| X043 + X280 + X415 | 0.778 | 0.702 | 0.752 | 0.6345 |
| X043 + X280 + X459 | 0.753 | 0.612 | 0.752 | 0.6498 |
| X043 + X280 + X473 | 0.781 | 0.679 | 0.752 | 0.6622 |
| X043 + X280 + X508 | 0.766 | 0.67 | 0.752 | 0.648 |
| X043 + X280 + X511 | 0.761 | 0.677 | 0.752 | 0.6438 |
| X043 + X280 + X513 | 0.759 | 0.642 | 0.752 | 0.6627 |
| X043 + X280 + X531 | 0.757 | 0.619 | 0.752 | 0.6626 |
| X043 + X280 + X544 | 0.752 | 0.634 | 0.752 | 0.6495 |
| X043 + X286 + X407 | 0.75 | 0.649 | 0.752 | 0.6577 |
| X043 + X286 + X415 | 0.761 | 0.595 | 0.752 | 0.6664 |
| X043 + X286 + X473 | 0.792 | 0.685 | 0.752 | 0.6624 |
| X043 + X286 + X513 | 0.759 | 0.642 | 0.752 | 0.6639 |
| X043 + X289 + X415 | 0.754 | 0.597 | 0.752 | 0.6583 |
| X043 + X289 + X473 | 0.78 | 0.679 | 0.752 | 0.6589 |
| X043 + X293 + X407 | 0.757 | 0.615 | 0.752 | 0.6454 |
| X043 + X293 + X415 | 0.769 | 0.63 | 0.752 | 0.6534 |
| X043 + X293 + X473 | 0.792 | 0.704 | 0.752 | 0.6508 |
| X043 + X293 + X508 | 0.757 | 0.634 | 0.752 | 0.648 |
| X043 + X293 + X513 | 0.753 | 0.632 | 0.752 | 0.6477 |
| X043 + X293 + X531 | 0.75 | 0.623 | 0.752 | 0.652 |
| X043 + X401 + X415 | 0.75 | 0.606 | 0.752 | 0.6627 |
| X043 + X401 + X473 | 0.779 | 0.681 | 0.752 | 0.6618 |
| X043 + X407 + X415 | 0.766 | 0.642 | 0.752 | 0.6537 |
| X043 + X407 + X473 | 0.786 | 0.69 | 0.752 | 0.6573 |
| X043 + X407 + X508 | 0.758 | 0.621 | 0.752 | 0.6456 |
| X043 + X407 + X511 | 0.753 | 0.619 | 0.752 | 0.6494 |
| X043 + X407 + X513 | 0.755 | 0.651 | 0.752 | 0.6384 |
| X043 + X415 + X473 | 0.803 | 0.73 | 0.752 | 0.6388 |
| X043 + X415 + X508 | 0.763 | 0.625 | 0.752 | 0.6588 |
| X043 + X415 + X511 | 0.755 | 0.597 | 0.752 | 0.6643 |
| X043 + X415 + X513 | 0.753 | 0.587 | 0.752 | 0.6813 |
| X043 + X415 + X531 | 0.75 | 0.625 | 0.752 | 0.6631 |
| X043 + X415 + X544 | 0.752 | 0.625 | 0.752 | 0.6569 |
| X043 + X459 + X473 | 0.779 | 0.677 | 0.752 | 0.6638 |
| X043 + X473 + X508 | 0.79 | 0.7 | 0.752 | 0.659 |
| X043 + X473 + X511 | 0.786 | 0.681 | 0.752 | 0.6571 |
| X043 + X473 + X513 | 0.78 | 0.672 | 0.752 | 0.6695 |
| X043 + X473 + X531 | 0.787 | 0.702 | 0.752 | 0.6604 |
| X043 + X473 + X544 | 0.779 | 0.685 | 0.752 | 0.6614 |
| X043 + X508 + X513 | 0.75 | 0.632 | 0.752 | 0.6449 |
| X066 + X082 + X166 | 0.763 | 0.634 | 0.752 | 0.6541 |
| X066 + X082 + X278 | 0.778 | 0.672 | 0.752 | 0.6571 |
| X066 + X082 + X473 | 0.789 | 0.694 | 0.752 | 0.6568 |
| X066 + X082 + X508 | 0.811 | 0.79 | 0.752 | 0.6088 |
| X066 + X278 + X473 | 0.759 | 0.657 | 0.752 | 0.6988 |
| X082 + X166 + X188 | 0.764 | 0.655 | 0.752 | 0.6519 |
| X082 + X166 + X278 | 0.793 | 0.681 | 0.752 | 0.6597 |
| X082 + X166 + X280 | 0.785 | 0.687 | 0.752 | 0.656 |
| X082 + X166 + X286 | 0.771 | 0.655 | 0.752 | 0.6566 |
| X082 + X166 + X289 | 0.766 | 0.642 | 0.752 | 0.6587 |
| X082 + X166 + X293 | 0.768 | 0.64 | 0.752 | 0.6564 |
| X082 + X166 + X401 | 0.761 | 0.636 | 0.752 | 0.6596 |
| X082 + X166 + X407 | 0.778 | 0.685 | 0.752 | 0.6485 |
| X082 + X166 + X415 | 0.786 | 0.666 | 0.752 | 0.6487 |
| X082 + X166 + X459 | 0.764 | 0.645 | 0.752 | 0.6584 |
| X082 + X166 + X473 | 0.812 | 0.722 | 0.752 | 0.6557 |
| X082 + X166 + X508 | 0.78 | 0.677 | 0.752 | 0.6546 |
| X082 + X166 + X511 | 0.765 | 0.636 | 0.752 | 0.659 |
| X082 + X166 + X513 | 0.788 | 0.666 | 0.752 | 0.6519 |
| X082 + X166 + X531 | 0.776 | 0.657 | 0.752 | 0.657 |
| X082 + X166 + X544 | 0.773 | 0.672 | 0.752 | 0.6444 |
| X082 + X188 + X278 | 0.784 | 0.694 | 0.752 | 0.6485 |
| X082 + X188 + X473 | 0.793 | 0.7 | 0.752 | 0.6541 |
| X082 + X188 + X508 | 0.815 | 0.794 | 0.752 | 0.6108 |
| X082 + X278 + X280 | 0.791 | 0.692 | 0.752 | 0.6601 |
| X082 + X278 + X286 | 0.784 | 0.666 | 0.752 | 0.6601 |
| X082 + X278 + X289 | 0.779 | 0.679 | 0.752 | 0.6539 |
| X082 + X278 + X293 | 0.782 | 0.702 | 0.752 | 0.645 |
| X082 + X278 + X401 | 0.783 | 0.67 | 0.752 | 0.6615 |
| X082 + X278 + X407 | 0.781 | 0.698 | 0.752 | 0.65 |
| X082 + X278 + X415 | 0.802 | 0.707 | 0.752 | 0.6363 |
| X082 + X278 + X459 | 0.779 | 0.662 | 0.752 | 0.6611 |
| X082 + X278 + X473 | 0.822 | 0.752 | 0.752 | 0.6428 |
| X082 + X278 + X508 | 0.793 | 0.675 | 0.752 | 0.6639 |
| X082 + X278 + X511 | 0.777 | 0.662 | 0.752 | 0.6589 |
| X082 + X278 + X513 | 0.795 | 0.719 | 0.752 | 0.6366 |
| X082 + X278 + X531 | 0.78 | 0.67 | 0.752 | 0.6541 |
| X082 + X278 + X544 | 0.784 | 0.666 | 0.752 | 0.6541 |
| X082 + X280 + X286 | 0.767 | 0.649 | 0.752 | 0.6628 |
| X082 + X280 + X407 | 0.754 | 0.625 | 0.752 | 0.6471 |
| X082 + X280 + X415 | 0.776 | 0.681 | 0.752 | 0.617 |
| X082 + X280 + X473 | 0.79 | 0.67 | 0.752 | 0.664 |
| X082 + X280 + X508 | 0.787 | 0.687 | 0.752 | 0.6431 |
| X082 + X280 + X513 | 0.756 | 0.606 | 0.752 | 0.6387 |
| X082 + X286 + X293 | 0.75 | 0.655 | 0.752 | 0.6402 |
| X082 + X286 + X407 | 0.757 | 0.634 | 0.752 | 0.6471 |
| X082 + X286 + X415 | 0.774 | 0.662 | 0.752 | 0.6278 |
| X082 + X286 + X473 | 0.807 | 0.73 | 0.752 | 0.6378 |
| X082 + X286 + X508 | 0.764 | 0.675 | 0.752 | 0.6414 |
| X082 + X286 + X513 | 0.783 | 0.672 | 0.752 | 0.6384 |
| X082 + X286 + X531 | 0.757 | 0.593 | 0.752 | 0.6587 |
| X082 + X289 + X415 | 0.76 | 0.63 | 0.752 | 0.6042 |
| X082 + X289 + X473 | 0.79 | 0.69 | 0.752 | 0.6562 |
| X082 + X289 + X508 | 0.778 | 0.7 | 0.752 | 0.6217 |
| X082 + X289 + X513 | 0.755 | 0.625 | 0.752 | 0.6262 |
| X082 + X293 + X415 | 0.762 | 0.647 | 0.752 | 0.6038 |
| X082 + X293 + X473 | 0.796 | 0.69 | 0.752 | 0.6618 |
| X082 + X293 + X508 | 0.777 | 0.713 | 0.752 | 0.6247 |
| X082 + X401 + X473 | 0.789 | 0.683 | 0.752 | 0.6585 |
| X082 + X401 + X508 | 0.775 | 0.681 | 0.752 | 0.6243 |
| X082 + X407 + X415 | 0.759 | 0.61 | 0.752 | 0.6146 |
| X082 + X407 + X473 | 0.796 | 0.7 | 0.752 | 0.6588 |
| X082 + X407 + X508 | 0.826 | 0.803 | 0.752 | 0.6176 |
| X082 + X415 + X473 | 0.812 | 0.73 | 0.752 | 0.6329 |
| X082 + X415 + X508 | 0.785 | 0.651 | 0.752 | 0.6098 |
| X082 + X415 + X511 | 0.751 | 0.63 | 0.752 | 0.6128 |
| X082 + X415 + X513 | 0.75 | 0.597 | 0.752 | 0.6271 |
| X082 + X459 + X473 | 0.79 | 0.687 | 0.752 | 0.6588 |
| X082 + X459 + X508 | 0.774 | 0.657 | 0.752 | 0.6238 |
| X082 + X473 + X508 | 0.806 | 0.722 | 0.752 | 0.6499 |
| X082 + X473 + X511 | 0.794 | 0.679 | 0.752 | 0.659 |
| X082 + X473 + X513 | 0.795 | 0.681 | 0.752 | 0.6538 |
| X082 + X473 + X531 | 0.798 | 0.687 | 0.752 | 0.6542 |
| X082 + X473 + X544 | 0.792 | 0.664 | 0.752 | 0.6658 |
| X082 + X508 + X511 | 0.768 | 0.653 | 0.752 | 0.6255 |
| X082 + X508 + X513 | 0.772 | 0.655 | 0.752 | 0.6189 |
| X082 + X508 + X531 | 0.76 | 0.636 | 0.752 | 0.6262 |
| X082 + X508 + X544 | 0.832 | 0.818 | 0.752 | 0.6 |
| X166 + X278 + X415 | 0.755 | 0.64 | 0.752 | 0.7062 |
| X166 + X278 + X473 | 0.774 | 0.67 | 0.752 | 0.6934 |
| X166 + X278 + X513 | 0.75 | 0.642 | 0.752 | 0.7006 |
| X166 + X286 + X473 | 0.757 | 0.63 | 0.752 | 0.7109 |
| X166 + X407 + X473 | 0.756 | 0.64 | 0.752 | 0.7081 |
| X166 + X415 + X473 | 0.768 | 0.647 | 0.752 | 0.709 |
| X166 + X473 + X508 | 0.758 | 0.66 | 0.752 | 0.7076 |
| X166 + X473 + X511 | 0.752 | 0.653 | 0.752 | 0.7036 |
| X166 + X473 + X513 | 0.756 | 0.619 | 0.752 | 0.7167 |
| X166 + X473 + X531 | 0.757 | 0.617 | 0.752 | 0.7109 |
| X166 + X473 + X544 | 0.752 | 0.606 | 0.752 | 0.7167 |
| X188 + X278 + X473 | 0.761 | 0.651 | 0.752 | 0.7053 |
| X278 + X280 + X415 | 0.751 | 0.625 | 0.752 | 0.7055 |
| X278 + X280 + X473 | 0.762 | 0.657 | 0.752 | 0.7127 |
| X278 + X286 + X473 | 0.775 | 0.64 | 0.752 | 0.7078 |
| X278 + X286 + X513 | 0.751 | 0.619 | 0.752 | 0.7162 |
| X278 + X289 + X473 | 0.761 | 0.653 | 0.752 | 0.7057 |
| X278 + X293 + X473 | 0.761 | 0.681 | 0.752 | 0.6949 |
| X278 + X401 + X473 | 0.771 | 0.67 | 0.752 | 0.7059 |
| X278 + X407 + X473 | 0.764 | 0.662 | 0.752 | 0.7038 |
| X278 + X415 + X473 | 0.781 | 0.694 | 0.752 | 0.6858 |
| X278 + X459 + X473 | 0.763 | 0.657 | 0.752 | 0.7003 |
| X278 + X473 + X508 | 0.776 | 0.668 | 0.752 | 0.7071 |
| X278 + X473 + X511 | 0.762 | 0.655 | 0.752 | 0.7003 |
| X278 + X473 + X513 | 0.767 | 0.668 | 0.752 | 0.7036 |
| X278 + X473 + X531 | 0.768 | 0.638 | 0.752 | 0.7146 |
| X278 + X473 + X544 | 0.768 | 0.651 | 0.752 | 0.7084 |
| X286 + X407 + X473 | 0.751 | 0.64 | 0.752 | 0.6992 |
| X286 + X415 + X473 | 0.773 | 0.636 | 0.752 | 0.7106 |
| X286 + X473 + X508 | 0.757 | 0.675 | 0.752 | 0.6957 |
| X286 + X473 + X513 | 0.763 | 0.675 | 0.752 | 0.6894 |
| X286 + X473 + X531 | 0.764 | 0.638 | 0.752 | 0.7076 |
| X293 + X415 + X473 | 0.752 | 0.645 | 0.752 | 0.6938 |
| X407 + X415 + X473 | 0.753 | 0.655 | 0.752 | 0.6933 |
| X415 + X473 + X508 | 0.759 | 0.66 | 0.752 | 0.6959 |
| X415 + X473 + X511 | 0.755 | 0.668 | 0.752 | 0.6846 |

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Similarly, it should be appreciated that in the foregoing description of embodiments of the present disclosure, various features are sometimes grouped together in a single embodiment, figure, or description thereof to streamline the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

What is claimed is:

1. A system for detecting colorectal cancer or advanced colorectal adenoma (CRC/ACRA) in a subject, comprising:
at least one storage device including a set of instructions; and
at least one processor in communication with the at least one storage device, wherein when executing the set of instructions, the at least one processor is directed to perform operations including:
(a) obtaining, from a quantitative measurement device, quantified abundance of one or more target metabolites in a panel of a plurality of metabolites in a sample from the subject, wherein the plurality of metabolites include the metabolites of Table A:
TABLE A

| Meta | | MASS | Delta | Meta | | MASS | Delta |
|---|---|---|---|---|---|---|---|
| ID | Compound | (+/−) | (ppm) | ID | Compound | (−/+) | (ppm) |
| BN001 | 5_HETE | 319.228(−) | 0 | C135 | C8H10N4O2 | 195.087(+) | 2 |
| BN002 | 15_HETE | 319.228(−) | 0 | C145 | C23H46NO6P | 464.314(+) | 2 |
| BN003 | 8_HETE | 319.228(−) | 0 | C150 | C27H48NO7P | 530.324(+) | 1 |
| BN005 | 11_HETE | 319.228(−) | 0 | DS09 | TCA (Taurocholic | 514.693(−) | 0 |
| | | | | | Acid) | | |
| BN006 | 12_HETE | 319.228(−) | 0 | DS11 | 5β-CAA-3β,12α-2K | 391.300(−) | 0 |
| BN015 | Sphingosine-1-phosphate (d16:1) | 350.210(−) | 0 | X006 | C13H16N2O4 | 263.104(−) | 1 |
| BN016 | Octadecane dioic acid | 313.239(−) | 0 | X043 | C20H38O11 | 453.232(−) | 5 |
| BN017 | Epitestosterone Sulfate | 367.300(−) | 0 | X082 | C19H26N6O | 353.212(−) | 7 |
| BN018 | (9E,11E)-13-Hydroperoxy-9,11- | 311.100(−) | 0 | X166 | C16H34NO5P | 352.224(+) | 2 |
| | octadecadienoic acid | | | | | | |
| BN029 | Azelaic acid | 187.098(−) | 0 | X188 | C28H48NO7P | 542.324(+) | 0 |
| BN030 | Arachidonic acid | 303.233(−) | 0 | X278 | C11H18N2O7 | 289.106(−) | 6 |
| BN035 | 9(S),10(S),13(S)_Trihydroxy_{—} | 329.234(−) | 0 | X280 | C29H46O | 447.312(−) | 19 |
| | 11(E)_Octadecenoic Acid | | | | | | |
| BP001 | 3-Indolepropionic acid | 190.086(+) | 0 | X286 | C29H49NO5 | 512.336(−) | 1 |
| BP010 | trans-2-Hexadecenoyl-L-carnitine | 398.326(+) | 0 | X289 | C31H41NO4 | 536.299(−) | 5 |
| BN012 | 14(S)-HDHA | 343.228(−) | 0 | X293 | C11H11NO3 | 204.067(−) | 2 |
| BN032 | Chenodeoxycholic Acid-3- | 471.235(−) | 0 | X407 | C17H17NO5 | 314.103(−) | 1 |
| | Sulfate Sodium Salt | | | | | | |
| BP002 | S-(−)-Cotinine | 177.102(+) | 0 | X415 | C20H34O6 | 369.227(−) | 3 |
| BP003 | 2-Arachidonoyl Glycerol | 379.284(+) | 0 | X473 | C28H50O5 | 465.358(−) | 1 |
| BP009 | 1-Linoleoyl-rac-glycerol | 355.284(+) | 0 | X508 | C7H2F5NO | 212.020(+) | 33 |
| BP01 | 17-Alpha-Methyltestosterone | 303.232(+) | 0 | X513 | C20H32O2 | 305.247(+) | 2 |
| C002 | C13H10O5 | 245.050(−) | 17 | X531 | C52H97NO18 | 1024.676(+) | 2 |
| C037 | C7H10OS3 | 204.982(−) | 1 | \ | \ | \ | \; |

(b) determining a sample score by processing the quantified abundance of each of the one or more target metabolites using a prediction model; and
(c) estimating whether the subject has CRC/ACRA by comparing the sample score to a cut-off score.

2. The system of claim 1, wherein the one or more target metabolites include at least two, three, or four metabolites in Table A.

3. The system of claim 1, wherein the one or more target metabolites include at least ten, fifteen, or twenty metabolites in Table A.

4. The system of claim 1, wherein the one or more target metabolites include all the metabolites in Table A.

5. The system of claim 1, wherein the plurality of metabolites further include metabolites in Table B:
TABLE B

| Meta | | MASS | Delta | Meta | | MASS | Delta |
|---|---|---|---|---|---|---|---|
| ID | Compound | (+/−) | (ppm) | ID | Compound | (−/+) | (ppm) |
| BN010 | 9(10)_DiHOME | 313.239(−) | 0 | C118 | C22H34O2 | 331.262(+) | 1 |
| BN011 | 12(13)_DiHOME | 313.239(−) | 0 | C119 | C21H36O3 | 337.273(+) | 2 |
| BN031 | 3-Hydroxynonanoic acid | 173.119(−) | 0 | C138 | C16H31NO4 | 302.233(+) | 2 |
| BN031 | 1-Palmitoyl-2-hydroxy-sn-glycero-3-PE | 452.279(−) | 0 | DS01 | CA (CholicAcid) | 407.281(−) | 0 |
| BP006 | Myristoyl-L-carnitine | 372.311(+) | 0 | DS02 | CDCA (Chenodeoxycholic | 391.286(−) | 0 |
| | | | | | Acid) | | |
| BN019 | 9(S),10(S),13(S)-Trihydroxy-10(E)- | 329.234(−) | 0 | DS03 | DCA (Deoxycholic Acid) | 391.286(−) | 0 |
| | Octadecenoic Acid | | | | | | |
| BN020 | 3α-Hydroxy-6-OXO-5α-Cholan- | 389.270(−) | 0 | DS04 | GCA (Glycocholic Acid | 464.302(−) | 0 |
| | 24-OIC Acid | | | | Hydrate) | | |
| BN021 | 3-Dehydrocholic Acid | 405.265(−) | 0 | DS05 | GCDCA (Glycochenodeoxycholic | 448.307(−) | 0 |
| | | | | | Acid) | | |
| BN022 | 5α-Cholanic Acid-3α,7β-Diol-6-One | 405.265(−) | 0 | DS06 | GDCA (Glycodeoxycholic Acid) | 448.307(−) | 0 |
| BN023 | Eicosapentaenoic Acid | 301.218(−) | 0 | DS07 | GLCA (Glycolithocholic Acid) | 432.312(−) | 0 |
| BN025 | Hydrocortisone | 361.202(−) | 0 | DS08 | GUDCA (Glycoursodeoxycholic | 448.307(−) | 0 |
| | | | | | Acid) | | |
| BN027 | 1-Stearoyl-2-Hydroxy-sn-Glycero-3- | 480.310(−) | 0 | DS10 | UDCA (Ursodeoxycholic Acid) | 391.286(−) | 0 |
| | Phosphoethanolamine | | | | | | |
| BN028 | Pimelic acid | 159.067(−) | 0 | X042 | C21H44NO7P | 452.278(−) | 1 |
| BP007 | trans-2-octenoyl-I-carnitine | 286.201(+) | 0 | X066 | C29H56NO9P | 592.362(−) | 0 |
| BP008 | 2-Methylbutyryl-L-carnitine | 246.170(+) | 0 | X401 | C12H17NO3 | 222.114(−) | 2 |
| BP011 | Decanoyl-L-carnitine | 316.248(+) | 0 | X459 | C43H73N3O16P2 | 930.431(−) | 3 |
| BP012 | (±)-Hexanoyl carnitine chloride | 261.193(+) | 0 | X511 | C17H26O2 | 263.200(+) | 2 |
| C004 | C10H12N4O5 | 267.073(−) | 2 | X544 | C26H29NO9 | 500.170(+) | 43 |
| BN004 | 9_HETE | 319.228(−) | 0 | C008 | C19H36O4 | 327.256(−) | 5 |

wherein the one or more target metabolites include at least one metabolite in Table A and at least one metabolite in Table B.

6. The system of claim 5, wherein the one or more target metabolites include one metabolite in Table A and one metabolite in Table B.

7. The system of claim 5, wherein the one or more target metabolites include one metabolite in Table A and two metabolites in Table B.

8. The system of claim 5, wherein the one or more target metabolites include two metabolites in Table A and one metabolite in Table B.

9. The system of claim 1, wherein the plurality of metabolites further metabolites in Table C:
TABLE C

| Meta | | MASS | Delta |
|---|---|---|---|
| ID | Compound | (+/−) | (ppm) |
| C001 | C12H22O4 | 229.144(−) | 2 |
| C005 | C16H32O3 | 271.228(−) | 2 |
| C009 | C22H20N4O | 355.158(−) | 4 |
| C011 | C16H30O10 | 381.174(−) | 7 |
| C012 | C19H21N5O3S | 398.132(−) | 7 |
| C014 | C21H24FN3O2S | 400.148(−) | 5 |
| C016 | C24H23F3N2O2 | 427.163(−) | 2 |
| C017 | C27H52O4 | 439.379(−) | 0 |
| C019 | C27H44O5 | 447.312(−) | 1 |
| C020 | C25H38O8 | 465.249(−) | 1 |
| C022 | C20H38O12 | 469.227(−) | 4 |
| C023 | C21H34N4O8 | 469.227(−) | 7 |
| C027 | C27H48O4 | 481.354(−) | 1 |
| C028 | C24H45O9P | 507.273(−) | 0 |
| C029 | C29H33N7O2 | 510.253(−) | 18 |
| C030 | C27H48NO7P | 528.310(−) | 1 |
| C035 | C33H45N5O5 | 590.346(−) | 19 |
| C036 | C31H57N5O9 | 642.396(−) | 19 |
| C038 | C18H32O2 | 279.233(−) | 0 |
| C040 | C22H34O2 | 329.248(−) | 0 |
| C041 | C26H44O9 | 499.288(−) | 7 |
| C102 | C7H8N4O2 | 181.072(+) | 1 |
| C108 | C15H26O4 | 271.190(+) | 1 |
| C110 | C15H27NO4 | 286.201(+) | 1 |
| C112 | C17H18N2O4 | 315.134(+) | 0 |
| C115 | C16H28O6 | 317.195(+) | 3 |
| C121 | C20H39NO4 | 358.295(+) | 1 |
| C122 | C24H37NO2 | 372.300(+) | 28 |
| C124 | C23H43NO4 | 398.325(+) | 4 |
| C127 | C25H47NO4 | 426.357(+) | 2 |
| C129 | C25H47NO5 | 442.352(+) | 2 |
| C130 | C21H42NO7P | 452.277(+) | 0 |
| C131 | C22H46NO7P | 468.308(+) | 1 |
| C136 | C19H26O2 | 287.204(+) | 14 |
| C144 | C24H36O2 | 357.280(+) | 3 |
| C146 | C23H48NO7P | 482.324(+) | 0 |
| C148 | C25H50NO7P | 508.340(+) | 1 |
| X004 | C12H16O5 | 239.092(−) | 2 |
| X010 | C17H34O | 299.259(−) | 1 |
| X012 | C17H18N2O4 | 313.119(−) | 1 |
| X013 | C18H34O4 | 313.238(−) | 1 |
| X016 | C20H32O3 | 319.228(−) | 0 |
| X023 | C19H28O5S | 367.158(−) | 1 |
| X024 | C19H30O5S | 369.174(−) | 0 |
| X032 | C25H24O5 | 403.158(−) | 7 |
| X036 | C23H27FN4O3 | 425.201(−) | 4 |
| X055 | C23H48NO7P | 526.315(−) | 0 |
| X068 | C32H51NO11 | 624.339(−) | 0 |
| X070 | C34H66NO8P | 646.427(−) | 28 |
| X133 | C7H10O3 | 143.07(+) | 2 |
| X139 | C11H20O4 | 217.143(+) | 2 |
| X141 | C11H22N2O3 | 231.174(+) | 16 |
| X144 | C17H33NO4 | 257.174(+) | 3 |
| X152 | C18H25N3O | 300.216(+) | 30 |
| X155 | C20H30O2 | 303.231(+) | 3 |
| X183 | C13H17N3O | 490.300(+) | 5 |
| X196 | C21H24N4O2 | 347.196(+) | 25 |
| X281 | C50H89O11P | 447.312(−) | 27 |
| X285 | C28H47NO6 | 512.336(−) | 5 |
| X292 | C7H8O4S | 187.007(−) | 0 |
| X314 | C32H31O14 | 637.157(−) | 0 |
| X402 | C10H18O2 | 229.144(−) | 2 |
| X403 | C13H12N4O | 239.092(−) | 8 |
| X405 | C17H30 | 279.233(−) | 0 |
| X409 | C20H34O | 335.259(−) | 0 |
| X412 | C19H22N4O3 | 353.164(−) | 6 |
| X419 | C12H22S2 | 229.108(−) | 4 |
| X426 | C10H18O4 | 201.113(−) | 1 |
| X429 | C22H22O10 | 445.114(−) | 0 |
| X431 | C23H34N4O5 | 445.244(−) | 4 |
| X432 | C10H16O3 | 183.103(−) | 2 |
| X433 | C11H14N4 | 201.113(−) | 8 |
| X434 | C11H24N6O3 | 287.186(−) | 8 |
| X435 | C11H22O3 | 201.150(−) | 2 |
| X436 | C20H34O8 | 401.218(−) | 0 |
| X437 | C16H30O5 | 301.202(−) | 0 |
| X448 | C18H34O5 | 329.233(−) | 1 |
| X449 | C39H61O8P | 687.398(−) | 7 |
| X451 | C41H65O8P | 715.429(−) | 8 |
| X454 | C35H61O10P | 671.402(−) | 13 |
| X455 | C37H67O9P | 685.419(−) | 38 |
| X456 | C38H69O9P | 699.434(−) | 38 |
| X457 | C28H48O5 | 463.343(−) | 0 |
| X458 | C18H36O3 | 299.259(−) | 1 |
| X463 | C9H14O | 183.103(−) | 2 |
| X470 | C37H68NO9P | 700.436(−) | 28 |
| X471 | C39H71O9P | 713.450(−) | 37 |
| X509 | C13H20O3 | 225.148(+) | 2 |
| X512 | C18H30O | 263.236(+) | 4 |
| X515 | C19H18N4O2 | 335.151(+) | 2 |
| X517 | C18H24NO4P | 350.153(+) | 4 |
| X519 | C13H14O11 | 364.084(+) | 9 |
| X523 | C26H48O9 | 505.336(+) | 2 |
| X525 | C34H58O6 | 563.427(+) | 6 |
| X535 | C13H21NO2 | 224.165(+) | 2 |
| X536 | C12H16O2 | 193.122(+) | 2 |
| X540 | C25H52NO4P | 484.348(+) | 10 |
| X541 | C14H22O4 | 255.159(+) | 0 |
| X542 | C13H18O2 | 207.138(+) | 0 |
| X543 | C24H30N8O | 447.259(+) | 6 |
| X546 | C14H22O3 | 271.190(+) | 1 |
| X547 | C15H28O5 | 289.201(+) | 0 |
| X549 | C12H26N2O7 | 311.183(+) | 6 |
| X550 | C33H45N5O5 | 630.306(+) | 1 |
| X555 | C20H32O7 | 385.222(+) | 0 |
| X556 | C27H33NO3 | 420.259(+) | 14 |
| X557 | C20H29N3O5S2 | 456.144(+) | 40 |
| X558 | C27H38O2 | 395.300(+) | 14 |
| X559 | C24H40O6S | 457.279(+) | 38 |
| X560 | C36H60O6 | 589.435(+) | 19 |
| X561 | C37H53O4 | 544.406(+) | 25 |
| X563 | C18H35NO4 | 271.190(+) | 1 |
| X565 | C35H64O9 | 629.447(+) | 24 |
| X566 | C32H54O5 | 602.448(+) | 7 |
| X567 | C29H54O7 | 514.395(+) | 28 |
| X569 | C34H61NO6 | 602.447(+) | 13 |
| X570 | C18H26O | 558.421(+) | 17 |
| X571 | C29H52O6 | 514.395(+) | 30 |
| X572 | C27H51NO3S | 470.369(+) | 6 |
| X573 | C25H40O4 | 405.301(+) | 3 |
| X574 | C40H78NO9P | 748.542(+) | 9 |
| X575 | C42H82NO10P | 792.568(+) | 9 |
| X576 | C43H65NO4 | 660.489(+) | 15 |
| X577 | C26H44O8 | 485.311(+) | 0 |
| X578 | C38H74NO8P | 704.516(+) | 9 |
| X579 | C37H58O6 | 599.437(+) | 11 |
| X580 | C38H64O6 | 617.467(+) | 17 |
| X581 | C35H66NO8P | 660.489(+) | 44 |
| X582 | C28H46N2O6 | 507.336(+) | 14 |
| X583 | C36H61NO4 | 572.437(+) | 53 |
| X584 | C29H51O5P | 511.384(+) | 57 |
| X585 | C16H26O2 | 528.411(+) | 2 |
| X586 | C38H65NO5 | 616.463(+) | 50 |
| X587 | C39H76NO8P | 718.531(+) | 10 |
| X588 | C29H48O3 | 528.411(+) | 8 |
| X589 | C39H73O8P | 701.505(+) | 9 |
| X591 | C37H72NO7P | 674.505(+) | 10 |
| X592 | C38H63NO6 | 630.479(+) | 10 |
| X593 | C37H63NO4 | 586.452(+) | 53 |
| X595 | C42H82NO9P | 776.573(+) | 9 |
| X596 | C28H56NO7P | 550.390(+) | 6 |
| X597 | C40H78NO8P | 732.547(+) | 9 |
| X598 | C31H46O2 | 514.377(+) | 22 |
| X599 | C38H74NO7P | 688.521(+) | 10 |
| X600 | C37H67O8P | 671.494(+) | 44 |
| X601 | C36H69NO8 | 644.494(+) | 24 |
| X602 | C34H54O5 | 543.398(+) | 12 |
| X603 | C31H52O6 | 521.384(+) | 1 |
| X605 | C50H88NO9P | 878.640(+) | 15 |
| X606 | C33H57O10P | 645.385(+) | 14 |
| X608 | C38H65NO4 | 600.468(+) | 51 |
| X609 | C41H80NO8P | 746.562(+) | 10 |
| X614 | C35H61O10P | 673.416(+) | 13 |
| X616 | C17H37N7O4 | 404.301(+) | 7 |
| X617 | C36H63O10P | 687.431(+) | 11 |
| X618 | C32H48O5 | 513.342(+) | 30 |
| X619 | C37H65O10P | 701.447(+) | 12 |
| X620 | C38H72NO9P | 718.474(+) | 39 |
| X621 | C26H40O3 | 401.290(+) | 37 |
| X624 | C27H40N4O4 | 485.311(+) | 3 |
| X628 | C31H54O9 | 571.384(+) | 0 |
| X629 | C38H67O10P | 715.463(+) | 12 |
| X633 | C23H40O5 | 397.295(+) | 0 |
| X634 | C25H38N2O3 | 415.305(+) | 23 |
| X635 | C27H45NO3 | 432.332(+) | 35 |
| X639 | C35H58O8 | 690.463(+) | 8 |

wherein the one or more target metabolites include at least one metabolite in Table A and at least one metabolite in Table C.

10. The system of claim 1, wherein the plurality of metabolites further include metabolites in Table D:
TABLE D

| Meta | | MASS | Delta |
|---|---|---|---|
| ID | Compound | (+/−) | (ppm) |
| C006 | C18H32O3 | 295.229(−) | 2 |
| C010 | C22H18FN3O2 | 374.132(−) | 3 |
| C013 | C21H36O5S | 399.221(−) | 0 |
| C015 | C24H40O5 | 407.280(−) | 0 |
| C021 | C29H43NO2S | 468.308(−) | 30 |
| C024 | C24H40O7S | 471.242(−) | 0 |
| C025 | C25H43N3O6 | 480.308(−) | 0 |
| C026 | C25H43N3O6 | 480.310(−) | 4 |
| C031 | C34H43N3O3 | 540.331(−) | 14 |
| C032 | C33H35N5O5 | 580.235(−) | 37 |
| C033 | C33H34N4O6 | 581.241(−) | 1 |
| C034 | C37H47NO5 | 584.357(−) | 32 |
| C042 | C31H44O6 | 511.302(−) | 9 |
| C043 | C24H49O9P | 511.302(−) | 4 |
| C105 | C12H23NO4 | 246.170(+) | 0 |
| C113 | C15H17F3N2O2 | 315.134(+) | 9 |
| C114 | C19H37NO6 | 317.195(+) | 3 |
| C116 | C18H35NO4 | 330.263(+) | 2 |
| C120 | C21H38O4 | 355.283(+) | 4 |
| C128 | C25H48O5 | 428.363(+) | 23 |
| C132 | C23H21N5O5S | 480.134(+) | 1 |
| C134 | C14H22N2O2 | 518.368(+) | 4 |
| C137 | C19H27NO2 | 302.215(+) | 11 |
| C139 | C21H40O3 | 341.306(+) | 2 |
| C140 | C23H36N2 | 341.306(+) | 31 |
| C141 | C23H30O3 | 355.227(+) | 1 |
| C142 | C20H24O2 | 357.243(+) | 0 |
| C143 | C21H31F3O | 357.243(+) | 9 |
| C147 | C25H47NO9 | 506.323(+) | 18 |
| C149 | C28H45NO7 | 508.340(+) | 26 |
| X011 | C18H32O4 | 311.223(−) | 1 |
| X019 | C22H32O3 | 343.228(−) | 0 |
| X051 | C32H43NO4 | 504.310(−) | 4 |
| X154 | C16H23N5O | 302.196(+) | 5 |
| X160 | C17H33NO4 | 316.247(+) | 4 |
| X178 | C26H43NO5 | 432.311(+) | 1 |
| X408 | C20H30O3 | 317.212(−) | 1 |
| X411 | C22H34O3 | 345.243(−) | 2 |
| X416 | C20H25NO2S2 | 374.132(−) | 18 |
| X421 | C10H14N5O6PS | 362.030(−) | 8 |
| X422 | C9H15NO2 | 168.103(−) | 0 |
| X423 | C9H18O3 | 173.118(−) | 2 |
| X424 | C9H11NO6S | 297.983(−) | 12 |
| X425 | C16H12Cl2FN5 | 362.030(−) | 22 |
| X427 | C25H26O4 | 389.181(−) | 13 |
| X428 | C13H19N5O | 260.158(−) | 24 |
| X430 | C21H36O9 | 431.228(−) | 2 |
| X438 | C22H32N4O4 | 415.234(−) | 3 |
| X439 | C23H28N8 | 415.234(−) | 6 |
| X440 | C31H40N2O3 | 487.291(−) | 12 |
| X441 | C24H34N2O5 | 429.249(−) | 22 |
| X442 | C28H32O13 | 575.175(−) | 4 |
| X443 | C28H31FN2O | 429.249(−) | 33 |
| X444 | C23H40O8 | 443.265(−) | 0 |
| X445 | C33H57O11P | 659.366(−) | 14 |
| X446 | C28H42O5 | 457.280(−) | 35 |
| X447 | C32H28F3N5O4 | 602.203(−) | 2 |
| X450 | C32H54O12 | 629.355(−) | 1 |
| X452 | C37H62O13 | 713.414(−) | 3 |
| X453 | C15H17N3 | 716.431(−) | 16 |
| X460 | C18H14 | 229.108(−) | 25 |
| X461 | C14H7F6N3O | 346.035(−) | 20 |
| X462 | C18H15N3O3S | 390.025(−) | 18 |
| X464 | C19H32O9 | 403.197(−) | 1 |
| X465 | C37H54O11 | 673.382(−) | 34 |
| X466 | C42H56O8 | 687.396(−) | 8 |
| X467 | C36H63O11P | 701.413(−) | 14 |
| X468 | C37H65O11P | 715.429(−) | 14 |
| X469 | C43H69O8P | 743.459(−) | 9 |
| X472 | C43H70NO8P | 758.481(−) | 6 |
| X474 | C41H75O10P | 757.477(−) | 34 |
| X475 | C39H69O10P | 727.464(−) | 12 |
| X476 | C45H73O8P | 771.493(−) | 5 |
| X477 | C45H76NO8P | 788.519(−) | 6 |
| X478 | C41H75N2O10P | 785.508(−) | 1 |
| X479 | C42H77N2O10P | 799.524(−) | 0 |
| X480 | C51H89O14P | 955.601(−) | 10 |
| X516 | C21H22O4 | 339.162(+) | 9 |
| X518 | C21H19FN4O | 363.162(+) | 1 |
| X532 | C25H40N4O5 | 477.307(+) | 0 |
| X533 | C9H12 | 121.101(+) | 1 |
| X534 | C13H22O4 | 243.159(+) | 0 |
| X537 | C12H19NO3 | 226.144(+) | 1 |
| X538 | C27H41N5O4 | 500.343(+) | 40 |
| X539 | C30H51NO6 | 544.369(+) | 15 |
| X545 | C15H24O3 | 253.180(+) | 1 |
| X548 | C22H27N | 306.227(+) | 18 |
| X551 | C19H32O5 | 341.232(+) | 1 |
| X552 | C14H22O3 | 239.164(+) | 1 |
| X553 | C23H36O7 | 425.253(+) | 1 |
| X554 | C11H18O4 | 215.128(+) | 1 |
| X562 | C27H41NO6 | 476.294(+) | 14 |
| X564 | C28H32N4O2 | 457.280(+) | 44 |
| X568 | C26H38N4O4 | 471.296(+) | 1 |
| X590 | C20H33NO14 | 512.207(+) | 19 |
| X594 | C44H86NO10P | 820.601(+) | 6 |
| X604 | C36H65NO13 | 720.453(+) | 0 |
| X607 | C31H51N9O7 | 662.411(+) | 19 |
| X610 | C38H69O10P | 717.442(+) | 37 |
| X611 | C41H68NO8P | 734.469(+) | 9 |
| X612 | C35H63O9P | 659.400(+) | 43 |
| X613 | C34H62NO10P | 676.426(+) | 11 |
| X615 | C26H48N6O8 | 573.363(+) | 4 |
| X622 | C39H69O11P | 745.473(+) | 11 |
| X623 | C40H71O11P | 759.489(+) | 11 |
| X625 | C40H74NO11P | 776.515(+) | 10 |
| X626 | C44H75O9P | 779.524(+) | 2 |
| X627 | C14H24O4 | 513.342(+) | 0 |
| X630 | C40H69O10P | 741.478(+) | 11 |
| X631 | C41H70NO8P | 812.400(+) | 4 |
| X632 | C14H26O2 | 227.200(+) | 2 |
| X636 | C41H73O11P | 773.505(+) | 11 |
| X637 | C42H75O11P | 787.520(+) | 10 |
| X638 | C18H32N8O4 | 425.253(+) | 21 |
| X640 | C41H73O9P | 741.478(+) | 38 |
| X641 | C9H16O2 | 157.122(+) | 2 |

wherein the one or more target metabolites include at least one metabolite in Table A and at least one metabolite in Table D.

11. The system of claim 1, wherein the quantified abundance of each of the one or more target metabolites is determined by the quantitative measurement device using a relative quantification approach or an absolute quantification approach.

12. The system of claim 1, wherein the sample score indicates a probability that the subject has CRC/ACRA.

13. The system of claim 1, wherein the prediction model is a trained machine-learning model.

14. The system of claim 13, wherein the trained machine-learning model is obtained by training a preliminary model using a plurality of training datasets, wherein
each of the plurality of training datasets includes quantified abundance of the one or more target metabolites of a reference sample from a reference subject and a label indicating whether or not the reference subject has CRC/ACRA.

15. The system of claim 14, wherein the label is a negative label or a positive label, wherein
the positive label indicates that the reference subject has CRC/ACRA, and
the negative label indicates that the reference subject is normal, or has a colorectal polyp, or has a normal colorectal adenoma.

16. The system of claim 1, wherein the CRC is early-stage CRC.

17. The system of claim 1, wherein the CRC is advanced CRC.

18. The system of claim 1, wherein the quantitative measurement device is a liquid chromatography mass spectrometry device.

19. A method of detecting colorectal cancer or advanced colorectal adenoma (CRC/ACRA) in a subject and treating the subject, comprising:
(a) obtaining, from a quantitative measurement device, quantified abundance of one or more target metabolites in a panel of a plurality of metabolites in a sample from the subject, wherein the plurality of metabolites include the metabolites of Table A;
(b) determining a sample score by processing the quantified abundance of each of the one or more target metabolites using a prediction model;
(c) determining whether the subject has CRC/ACRA by at least comparing the sample score to a cut-off score; and
(d) in response to determining that the subject has CRC/ACRA, applying a treatment to the subject, wherein the treatment includes colectomy, ostomy, radiotherapy, pharmacotherapy, or a surgery for removing the ACRA or a tumor in the subject.

20. A kit for detecting colorectal cancer or advanced colorectal adenoma (CRC/ACRA) in a subject, comprising one or more target metabolites in a panel of a plurality of metabolites, wherein the plurality of metabolites include the metabolites of Table A.
